(19) United States
(12) Patent Application Publication
(10) Pub. No.: US 2026/0337557 A1
(43) Pub. Date: Oct. 29, 2026

(54) HUMAN CORNEAL ORGANOIDS AND ANTIOXIDANT BIOMARKER FOR KERATOCONUS

(71) Applicant: New York University, New York, NY (US)

(72) Inventors: Shukti Chakravarti, New York, NY (US); Sean Ashworth, New York, NY (US); Madhuri Koduri, New York, NY (US); George Maiti, New York, NY (US)

(21) Appl. No.: 19/656,654

(22) Filed: Apr. 23, 2026

Related U.S. Application Data
(60) Provisional application No. 63/793,892, filed on Apr. 24, 2025.
(60) Provisional application No. 63/798,877, filed on May 2, 2025.
(60) Provisional application No. 63/800,788, filed on May 6, 2025.

(51) Int. Cl. C12N 5/079; A61K 35/30; A61P 27/02; C12Q 1/6883; G01N 33/68

(52) U.S. Cl. CPC C12N 5/0621; A61K 35/30; A61P 27/02; C12Q 1/6883; G01N 33/6893; C12N 2501/155; C12N 2501/999; C12N 2506/45; C12N 2533/90; C12Q 2600/158; G01N 2333/908; G01N 2800/16

(57) ABSTRACT

Compositions comprising human cornea organoids and components thereof and a method of diagnosing Keratoconus are described herein.

### CROSS-REFERENCE TO RELATED APPLICATIONS

This application claims priority to U.S. Provisional Application No. 63/793,892, filed on Apr. 24, 2025, U.S. Provisional Application No. 63/798,877, filed on May 2, 2025, and U.S. provisional application No. 63/800,788, filed on May 6, 2025, each of which is incorporated herein by reference in their entirety.

### STATEMENT REGARDING FEDERALLY SPONSORED RESEARCH OR DEVELOPMENT

This invention was made with government support under R01 EY026104 and R01 EY030917 awarded by the National Institutes of Health. The government has certain rights in the invention.

### BACKGROUND OF THE INVENTION

Treatment options for diseases affecting the cornea are limited.

There is a need in the art for compositions and methods for the treatment and detection of corneal disease. The present invention fulfills this need.

### SUMMARY OF THE INVENTION

In some aspects, the invention relates to a method for forming a human cornea organoid.

In some embodiments, the method comprises providing neural ectoderm lineage cells, culturing the neural ectoderm lineage cells in an optic vesicle induction medium to induce the formation of one or more optic vesicles, and culturing the one or more optic vesicles in a corneal induction medium comprising less than 100 nM Smoothened Agonist (SAG).

In some embodiments, the optic vesicle induction medium comprises less than 100 nM SAG, less than 50 nM SAG, or about 0 nM SAG.

In some embodiments, the corneal induction medium comprises less than 50 nM SAG or about 0 nM SAG.

In some embodiments, the corneal induction medium comprises knockout serum replacement.

In some embodiments, the corneal induction medium comprises BMP-4.

In some embodiments, the method comprises the step of culturing the neural ectoderm lineage cells or the one or more optic vesicles in a maturation medium. In some embodiments, the maturation medium comprises knockout serum replacement.

In some embodiments, the neural ectoderm lineage cells are cultured in the optic vesicle induction medium for about 5 days.

In some embodiments, wherein the one or more optic vesicles are cultured in the corneal induction medium for about 5 days.

In some embodiments, the neural ectoderm lineage cells comprises culturing the neural ectoderm lineage cells in a low attachment plate.

In some embodiments, the neural ectoderm lineage cells are derived from induced pluripotent stem cells, embryonic stem cells, or somatic stem cells.

In some aspects, the invention provides a human cornea organoid formed, at least in part, by a method of the invention.

In some aspects, the invention relates to a composition comprising extracellular matrix derived from human cornea organoids.

In some embodiments, the extracellular matrix is derived from human cornea organoids formed, at least in part, by a method of the invention.

In some embodiments, the extracellular matrix comprises collagen I, collagen III, collagen V, fibronectin, glycosaminoglycans, keratan sulfate, or chondroitin sulfate.

In some embodiments, the invention relates to a scaffold comprising the composition.

In some embodiments, the extracellular matrix comprises a decellularized human cornea organoid.

In some embodiments, the composition comprises fibrin gel or polyethylene glycol diacrylate (PEGDA) hydrogel.

In some aspects, the invention relates to a composition comprising cells derived from human cornea organoid formed, at least in part, by a method of the invention.

In some embodiments, the cell comprise an epithelial cell, an epithelial-like cell, or a VEGFR1 negative cell.

In some aspects, the invention relates to a composition comprising stromal cells derived from a human cornea organoid.

In some embodiments, the human cornea organoids are formed, at least in part, by a method of the invention.

In some embodiments the stromal cells comprise a VEGFR1 positive cells.

In some embodiments, the stromal cells comprise keratocytes, progenitor keratocytes, differentiating keratocytes, keratocyte-like cells, keratocyte-like progenitor cells, proliferating keratocyte-like cells, immature keratocyte-like cells, fibroblasts, or myofibroblasts.

In some aspects, the invention relates to applying a composition comprising extracellular matrix or cells derived from a human corneal organoid to the eye of a subject.

In some embodiments, the eye of the subject has keratoconus, Fuchs endothelial dystrophy, infection-related corneal scarring, injury-related corneal scarring, a congenital corneal dystrophy, lattice corneal dystrophy, or Avellino corneal dystrophy.

In some embodiments, the method comprises the step of applying a fibrin sealant to the eye of the subject.

In some aspects, the invention relates to a cell culture substrate comprising a composition comprising extracellular matrix derived from a human corneal organoid.

In some aspects, the invention relates to a method of culturing cells in contact with a composition comprising extracellular matrix derived from a human corneal organoid.

In some embodiments, the cultured cells comprise primary cells from a subject.

In some embodiments, the primary cells from a subject comprise a corneal stromal cell or an epithelial stem cell.

In some embodiments, the cells are cultured on a cell culture substrate comprising a composition comprising extracellular matrix derived from a human corneal organoid.

In some aspects, the invention relates to a method for sorting human corneal organoid cells comprising the steps of contacting the human corneal organoid cells to a cell culture substrate comprising laminin, and collecting the human corneal organoid cells that are not adhered to the cell culture substrate.

In some embodiments, the cells adhered to the cell culture substrate are enriched for epithelial or epithelial-like cells.

In some embodiments, the collected cells are enriched for stromal cells.

In some aspects, the invention provides a method for diagnosing a subject as having, being at risk of having, or being at risk of developing Keratoconus (KC) comprising: a) measuring the level of an NRF2 target protein or RNA transcript encoding an NRF2 target protein in a biological sample from the subject; b) comparing the level of the NRF2 target protein or RNA transcript encoding an NRF2 target protein in the biological sample from the subject to the level of NRF2 target protein or RNA transcript encoding an NRF2 target protein in a comparator, wherein a differential level of NRF2 target protein or RNA transcript encoding an NRF2 target protein in the biological sample relative to the comparator indicates that the subject has, or is at risk for developing KC; and c) diagnosing the subject as having, being at risk of having, or being at risk of developing KC.

In some embodiments, the NRF2 target protein is one or more selected from the group consisting of glutathione peroxidase 3 (GPX3) and malondialdehyde (MDA). In some embodiments, the NRF2 target protein is GPX3.

In some embodiments, an increase in the level of the NRF2 target protein or RNA transcript encoding an NRF2 target protein in the biological sample relative to the comparator indicates that the subject has, or is at risk for developing KC.

In some embodiments, the biological sample is at least one selected from the group consisting of tear fluid, epithelial scraping, plasma, serum, and blood.

In some embodiments, the measuring the level of NRF2 target protein or RNA transcript encoding an NRF2 target protein in the biological sample from the subject comprises at least one technique selected from the group consisting of RT-PCR, RT-qPCR, ELISA, and immunofluorescence.

In some embodiments, the method further comprises a step of treating the subject to treat or prevent KC.

In some embodiments, the step of treating the subject comprises administering to the subject an effective amount of a therapeutic composition comprising an agent that reduces oxidative stress in the eye. In some embodiments, the therapeutic composition comprises sulforaphane.

In some embodiments, the step of treating the subject comprises administering to the subject an effective amount of a therapeutic composition that increases the expression of NRF2 or an NRF2 target protein in the eye.

In some embodiments, the NRF2 target protein is selected from HMOX1 and GPX3.

In some aspects, the invention relates to a method of treating Keratoconus (KC) in a subject, comprising administering a treatment to subject, wherein the subject has been identified as having an increased level of an NRF2 target protein or an RNA transcript encoding an NRF2 target protein in a biological sample.

In some embodiments, the subject has been identified as having an increased level of an NRF2 target protein or an RNA transcript encoding an NRF2 target protein in a biological sample compared to the level of an NRF2 target protein or RNA transcript encoding an NRF2 target protein in a comparator.

In some embodiments, the NRF2 target protein is one or more selected from the group consisting of GPX3 and MDA.

In some embodiments, the NRF2 target protein is GPX3.

In some embodiments, the biological sample is at least one selected from the group consisting of tear fluid, epithelial scraping, plasma, serum, and blood.

In some embodiments, the treatment comprises administering to the subject an effective amount of a therapeutic composition comprising an agent that reduces oxidative stress in the eye.

In some embodiments, the therapeutic composition comprises sulforaphane.

In some embodiments, the treatment comprises administering to the subject an effective amount of a therapeutic composition that increases the expression of NRF2 or an NRF2 target protein in the eye.

In some embodiments, the NRF2 target protein is selected from HMOX1 and GPX3.

### BRIEF DESCRIPTION OF THE DRAWINGS

For the purpose of illustrating the invention, there are depicted in the drawings certain embodiments of the invention. However, the invention is not limited to the precise arrangements and instrumentalities of the embodiments depicted in the drawings.

FIG. 1, comprising FIG. 1A and FIG. 1B, depicts data showing Human Corneal Organoid (HCO) clusters mimic immature corneal stroma. FIG. 1A depicts Uniform Manifold Approximation and Projection (UMAP) from merged scRNA-seq of three adult donor (DN) corneas and three 4-month-old HCOs. Details in Maiti, PNASnexus, 2022. FIG. 1B depicts differentially expressed genes from pseudo-bulked analysis showing active transcription of collagen genes in HCO clusters (CLs) 2, 5, and 10. The data shows that HCO stromal CL5 expresses proliferation genes, indicating its progenitor quality.

FIG. 2 depicts representative images of Cornea-like glycosaminoglycans (GAG) in HCO derived from the NCRM-1 induced pluripotent stem cell (iPSC) line. Chondroitin sulfate (CS), keratan sulfate (KS), and nuclear DAPI staining are shown. Scale bar=100 μm.

FIG. 3 depicts data showing HCOs resemble human fetal corneas. A heatmap of Area Under the Receiving Operating Characteristic (AUROC) scores between all cells present in corneal organoids and 20-21 post-conceptual week (PCW) fetal cornea. Stromal cell similarities marked (green). Detailed AUROC scores can be found in Ashworth, Ocul Surface, 2025.

FIG. 4, comprising FIG. 4A and FIG. 4B, depicts representative images of a decellularized HCO. FIG. 4A depicts a representative image of a human corneal organoid (HCO) derived from the H9 cell line at Day 106. FIG. 4B depicts a representative image of an HCO that was decellularized using a 1.5 M NaCl hypertonic solution for 20 hours, followed by PBS wash and DNase I treatment at 37 °C for 2 hours to remove nuclear material. The resulting extracellular matrix (ECM) was stored at −80 °C until further use.

FIG. 5, comprising FIG. 5A through FIG. 5C, depicts representative images of injected cells in decellularized DN corneas. FIG. 5A depicts a phase-contrast image of a DN cornea stained with DAPI (blue). FIG. 5B depicts a phase-contrast image of a DN cornea that was decellularized using 1.5 M NaCl for 48 hours, followed by PBS washes, DNase I digestion at 37 °C for 24 hours to remove nuclear material. Successful decellularization was evident from lack of DAPI+nuclei. FIG. 5C depicts a phase contrast image of decellularized cornea in which DN fibroblast cultures labeled with Vybrant DiO (green) were injected (20,000 cells per cornea) using a 31 g needle and maintained in fibroblast growth media (DMEM/F12 & 10% fetal bovine serum (FBS), 1× antibiotics) for 10 days. The recellularized cornea shows VyDiO+ cells (arrows). N=one cornea. Scale bar: 20 μm.

FIG. 6 depicts an exemplary protocol for generating human cornea organoids.

FIG. 7 depicts representative images of HCOs from both IMR90.4 and NCRM-1 iPS cell lines at different stages of development, shown at day 0, 11, 36 and 111. Scale bar represents 500 μm. Co, Re and RPE indicate presumptive cornea, retina and retinal pigment epithelial, respectively.

FIG. 8 depicts single cell RNA sequencing data of human cornea organoids.

FIG. 9 depicts representative immunofluorescence micrographs of human cornea organoids.

FIG. 10 depicts representative images of stained human cornea organoids.

FIG. 11 depicts representative immunofluorescence micrographs of human cornea organoids.

FIG. 12, comprising FIGS. 12A and 12B, depict representative 20× confocal images of cryosectioned HCO. FIG. 12A depicts cryosectioned HCO stained with alpha-smooth muscle actin (Invitrogen catalog number 14-9760-82) at a concentration of 1 μg/mL, in green, keratocan in magenta, and counterstained with DAPI nuclear stain. FIG. 12B depicts cryosectioned HCO stained with no primary antibodies as a primary control and counterstained with DAPI.

FIG. 13 depicts scRNA-seq data of human corneal organoids highlighting the gene expression of Ser/Thr kinases, Cyclin B1 and Cyclin B2.

FIG. 14 depicts a schematic of the TGF-β signaling network.

FIG. 15 depicts scRNA-seq data of human corneal organoids highlighting the gene expression of key TGF-β signal mediators.

FIG. 16 depicts a schematic of experiments to test ECM-regenerative TGF-β signals in HCOs.

FIG. 17 depicts an exemplary protocol for rapid extraction of human cornea organoid stromal cells.

FIG. 18 depicts an exemplary protocol for rapid extraction of human cornea organoid cells using magnetic beads.

FIG. 19 depicts representative images of donor cornea before and after injection of labeled fibroblasts.

FIG. 20 depicts representative images of donor cornea before and after integration of VEGFR1+ cells and VEGFR1-cells.

FIG. 21 depicts representative images of decellularized donor cornea before and after repopulation with fibroblasts.

FIG. 22 depicts representative experimental results of delivering fibroblasts to injured mouse corneas.

FIG. 23 depicts representative experimental results of delivering HCO derived ECM to mouse corneas.

FIG. 24 depicts representative images of decellularized HCO ECM, representative image of a cell migration assay, and representative experimental results of a cell migration assay.

FIG. 25 depicts representative images of extracellular matrix packing in human cornea organoids expressing only short form isoforms of Blimp1/Prdm1 transcription factors.

FIG. 26 depicts representative images of a 4-month old HCO derived from the NCRM-1 cell line stained for TUBB3 and counterstained with DAPI.

FIG. 27 depicts representative images of 4-month old HCO sections stained with anti-βIII tubulin and DAPI.

FIG. 28 depicts a schematic of an exemplary method for generating iPSC-derived human corneal organoids (HCOs) from donor or patient cells.

FIG. 29 depicts a schematic of an exemplary timeline for the differentiation of pluripotent stem cells to HCOs with key signaling molecules and changes highlighted in red.

FIG. 30 depicts representative images of HCOs from the NCRM-1 induced pluripotent stem (iPS) cell line grown in different concentrations of Smoothened agonist (SAG). Corneal (Co) and retinal (Re) regions of organoids are indicated. Scale bars represent 200 μm.

FIG. 31 depicts representative images of an HCO grown in fetal bovine serum and an HCO grown in Knockout Serum Replacement (KSR)-containing media at 3 months of culture. Scale bars are 1000 μm.

FIG. 32 depicts representative images of BLIMP 1 a wildtype (WT) and knockout (KO) two-month old corneal organoids stained with hematoxylin and eosin. Presumptive corneal stroma-like regions are indicated in boxes and shown as insets. The three principal corneal stromal-like layers are indicated-epithelium (Epi), stroma (Str) and endothelium (Endo). Arrowheads indicate corneal epithelial-like cells, which are well stratified in WT but appear disrupted in KO, including outside of cornea-like areas. Scale bars represent 200 μm.

FIG. 33 depicts a table showing selected gene normalized values (counts per million-CPM) with altered expression in BLIMP1a knockout corneal organoids. Downregulated genes are highlighted in orange and upregulated genes in green.

FIG. 34 depicts images of representative iPSC colonies from 4 patients that have keratoconus. Scale bars represent 100 μm.

FIG. 35 depicts a schematic of an exemplary method for generating human corneal organoids.

FIG. 36 depicts scRNA-seq data of human corneal organoids highlighting major corneal extracellular matrix (ECM) genes.

FIG. 37 depicts representative images of an infected an uninfected human corneal organoid.

FIG. 38 depicts representative immunofluorescence microscopy images of a human corneal organoid stained with a nuclear stain and to stained visualize KRT3 and COL5A1.

FIG. 39 depicts representative immunofluorescence microscopy images of a human corneal organoid stained with a nuclear stain and stained to visualize KRT3, HSV1, and CCL2.

FIG. 40 depicts representative immunofluorescence microscopy images of a human corneal organoids showing nerve-like structures.

FIG. 41 depicts images of wells of a 96 well plate containing human corneal organoids.

FIG. 42 depicts representative images of 11 day old human corneal organoids.

FIG. 43 depicts representative images of 22 day old human corneal organoids.

FIG. 44, comprising FIG. 44A-FIG. 44H, depicts the results of example experiments demonstrating the upregulation of GPX3 in Keratoconus tears and the source of GPX3 in tear fluid was tested in postnatal DO mice and GPX3 was rich in epithelial cells of ocular tissue. FIG. 44A depicts a comparison of GPX3 levels in tear fluid revealing a significant upregulation in the KC group compared to the non-KC group. FIG. 44B and FIG. 44D depict Kmax values plotted against GPX3 levels, revealing a positive correlation indicating an association with disease severity.

FIG. 44C depicts the results of example experiments demonstrating systemic GPX3 levels were elevated in the KC group compared to non-KC group individuals. FIG. 44E-FIG. 44G depict the results of example experiments demonstrating age, disease duration, and gender showed minimal correlation with GPX3 levels in tear fluid. FIG. 44H shows the eye of a P0 mouse with no GPX3 antibody control.

FIG. 44I depicts GPX3 expression in the epithelial tissues of eyelid skin, conjunctival epithelium, corneal epithelium, and lens epithelium (n=1).

FIG. 45, comprising FIG. 45A-FIG. 45C, depicts a schematic hypothesis of oxidative stress and NRF2 signaling corneal stromal degradation of Keratoconus disease. FIG. 45A shows that, in lipid peroxidation, reactive oxygen species (ROS) oxidize polyunsaturated fatty acids, undergoing lipid peroxidation which results in the production of malondialdehyde as an end-product. FIG. 45B shows that regulation of NRF2 occurs by KEAP1 release of NRF2, which then undergoes phosphorylation and translocation into the nucleus. There, it forms a heterodimer with MAFF, activating antioxidant responsive element (ARE) genes such as glutathione peroxidase (GPX), thioredoxin reductase (TXNRD1), and heme oxygenase-1 (HO-1). FIG. 45C depicts the glutathione metabolism cascade, wherein GPX3 plays a pivotal role in catalyzing the conversion of hydrogen peroxide into water.

FIG. 46 depicts the results of example experiments demonstrating MDA levels are significantly elevated in KC tears. KC tears were collected using a glass capillary tube and MDA levels were assayed using a sandwich ELISA. In the tear samples of donor normal (DN), MDA levels were found to be 57.58+/−142.91 (mean +/−SD) pmol/mL, while in KC tears, MDA levels were elevated to 204.36+/−118.37 pmol/mL (p<0.011).

FIG. 47 depicts the expression of NRF2 target genes in the corneal epithelial scrapings of KC and DN. Gene expression relative to GAPDH measured by Taqman qRT-PCR in total RNA from epithelial scraping of DN and KC (n=4). Peroxiredoxin I (PRDX1) is significantly upregulated (p<0.033) in KC, whereas MAF BZIP Transcription Factor F (MAFF) was decreased (p<0.0172). Expression of the other genes tested was not significantly different.

FIG. 48A depicts the experimental design of testing a cell culture model of oxidative stress and viability assay. FIG. 48B depicts the results of example experiments demonstrating that sulforaphane improved cell viability in fibroblasts treated with hydrogen peroxide.

FIG. 49 depicts the results of example experiments demonstrating subcellular localization of NRF2 and MAFF in a cell culture model of oxidative stress. DN and KC fibroblasts were immunostained for NRF2 and MAFF. In DN fibroblasts, the intensity of NRF2 increased in response to oxidative stress, while in the KC fibroblasts, the intensity was reduced. Similarly, the intensity of MAFF was lower in KC fibroblasts compared to DN fibroblasts.

FIG. 50 depicts the results of example experiments demonstrating NRF2 target gene expression in a cell culture model of oxidative stress. NRF2 target genes were evaluated before and after exposure to oxidative stress in DN and KC primary fibroblasts.

FIG. 51 depicts a model of NRF2 signaling under oxidative stress. Under homeostatic conditions, NRF2 is sequestered by KEAP1 and targeted for degradation, maintaining low antioxidant gene expression. Sulforaphane disrupts the NRF2-KEAP1 interaction, allowing NRF2 to accumulate, translocate to the nucleus, and activate antioxidant genes that protect against oxidative stress and ECM degradation, which is relevant to KC. In contrast, ML385 inhibits NRF2 by binding to its CNC-bZIP domain, preventing its interaction with MAFF and subsequent binding to AREs. This blocks downstream antioxidant gene activation, suppresses antioxidant defense, and promotes ECM breakdown, mimicking KC-like pathology. This model highlights how NRF2 modulation influences corneal fibroblast responses under oxidative stress.

FIG. 52 depicts the results of example experiments demonstrating the protective role of sulforaphane (SFN) in H₂O₂-treated corneal fibroblasts. Corneal fibroblasts were treated with varying H₂O₂ concentrations and 10 μM SFN. Cell viability and caspase 3/7 activity assays showed a concentration-dependent decrease in viability and increase in apoptosis with higher H₂O₂. SFN treatment protected cells, improving viability and reducing apoptosis, highlighting its protective role against oxidative stress.

FIG. 53 depicts the results of example experiments demonstrating sulforaphane mediated activation of NRF2 signaling in corneal stromal cells. Corneal fibroblasts from three biologically distinct DN and KC samples were treated with 10 μM SFN for 16 hours, followed by 50 μM H₂O₂ for 1 hour. NRF2 immunostaining was performed in untreated, H₂O₂-treated, and SFN+ H₂O₂-treated cells to assess NRF2 nuclear translocation. Gene expression analysis of GPX3, NRF2, and HMOX1 showed upregulation in response to SFN and oxidative stress, indicating enhanced NRF2-mediated antioxidant response in both DN and KC fibroblasts.

FIG. 54 depicts the results of example experiments demonstrating inhibition of NRF2 signaling in corneal stromal cells via ML385. The gene expression of NRF2 downstream target genes, including TXNRD1, was assessed in corneal stromal cells treated with ML385 (an NRF2 inhibitor). The results show reduced expression of these genes in ML385-treated cells compared to controls.

FIG. 55 depicts the results of example experiments demonstrating NRF2 inhibition reduces proliferation in corneal stromal cells. Brightfield images and cell counts of corneal stromal fibroblasts from three different biological donors are shown. Cells were treated with ML385 or DMSO (vehicle control, UT) and brightfield images were captured at D7 (after treatment). Cell counts on D7 revealed a reduction in cell number in ML385-treated cells compared to vehicle controls, indicating decreased proliferation over the 7-day period.

FIG. 56 depicts the results of example experiments demonstrating a decrease in fibronectin-1 after NRF2 inhibition. Cells were treated with ML385 or DMSO (vehicle control). Fibronectin-1 (FN1) staining revealed reduced staining in ML385-treated cells compared to controls, suggesting decreased ECM production. This indicates a potential role of NRF2, not directly, but through its effect on cell proliferation, which may disrupt ECM regulation.

FIG. 57 depicts the results of example experiments demonstrating a decrease in Col5A1 and Col1A1 after NRF2 inhibition. Cells were treated with ML385 or DMSO. Col5A1 staining showed reduced intensity in ML385-treated cells compared to controls, indicating decreased collagen deposition in the ECM. Gene expression analysis of Col5A1 and Col1A1 further confirmed reduced expression in ML385-treated cells, suggesting that NRF2 inhibition leads to downregulation of collagen production.

### DETAILED DESCRIPTION OF THE INVENTION

The present invention relates to human corneal organoids and methods of and methods of making compositions derived from any component of human corneal organoids. The present inventions also provides methods of using human corneal organoids and/or human corneal organoid derived compositions for any application. Applications may comprise treatment of a subject and/or any cell culture applications.

The present invention also relates to a method of diagnosing keratoconus (KC) or assessing the severity of KC in a subject based upon detection of the presence or relative level of a protein or RNA transcripts encoding a protein in a biological sample. In some embodiments, the method of diagnosing or assessing the severity of KC comprises measuring the level of an NRF2 target protein or RNA transcripts encoding an NRF2 target protein in a biological sample from a subject suspected of having, being at risk of having, or at risk of developing KC. In some embodiments, the NRF2 target protein is selected from glutathione peroxidase 3 (GPX3) and malondialdehyde (MDA).

In some embodiments, the level of the NRF2 target protein in the biological sample from the subject is measured using one or more techniques selected from the group consisting of enzyme-linked immunosorbent assay (ELISA, including “sandwich ELISA”), immunofluorescence, qRT-PCR, and Western blotting. In some embodiments, the biological sample is selected from tear fluid, epithelial cells, corneal stromal fibroblasts, plasma, or blood.

In some embodiments, the method further comprises a step of administering a treatment or therapy to the subject that has been diagnosed with having, being at risk of having, or being at risk of developing KC to treat or prevent KC.

### DEFINITIONS

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology can be used.

It is also to be understood that the terminology used herein is for the purpose of describing particular embodiments only, and is not intended to be limiting.

The articles “a” and “an” are used herein to refer to one or to more than one (i.e. to at least one) of the grammatical object of the article. By way of example, “an element” means one element or more than one element.

As used herein, the term “about” can be understood by persons of ordinary skill in the art and will vary to some extent on the context in which it is used. As used herein when referring to a measurable value such as an amount, a temporal duration, and the like, the term “about” is meant to encompass variations of +20%, +10%, +5%, +1%, or +0.1% from the specified value, as such variations are appropriate to perform the disclosed methods.

The term “abnormal” when used in the context of organisms, tissues, cells or components thereof, refers to those organisms, tissues, cells or components thereof that differ in at least one observable or detectable characteristic (e.g., age, treatment, time of day, etc.) from those organisms, tissues, cells or components thereof that display the “normal” (expected) respective characteristic. Characteristics which are normal or expected for one cell or tissue type, might be abnormal for a different cell or tissue type.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health. Preferably, the animal is a mammal. More preferably, the mammal is a human.

A disease or disorder is “alleviated” if the severity of a sign or symptom of the disease or disorder, the frequency with which such a sign or symptom is experienced by a patient, or both, is reduced.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

“Effective amount” refers to an amount of a compound, formulation, material, or composition, as described herein effective to achieve a particular biological result.

As used herein, the term “fragment,” as applied to a nucleic acid, refers to a subsequence of a larger nucleic acid. A “fragment” of a nucleic acid can be at least about 15 nucleotides in length; for example, at least about 50 nucleotides to about 100 nucleotides; at least about 100 to about 500 nucleotides, at least about 500 to about 1000 nucleotides; at least about 1000 nucleotides to about 1500 nucleotides; about 1500 nucleotides to about 2500 nucleotides; or about 2500 nucleotides (and any integer value in between). As used herein, the term “fragment,” as applied to a protein or peptide, refers to a subsequence of a larger protein or peptide. A “fragment” of a protein or peptide can be at least about 20 amino acids in length; for example, at least about 50 amino acids in length; at least about 100 amino acids in length; at least about 200 amino acids in length; at least about 300 amino acids in length; or at least about 400 amino acids in length (and any integer value in between).

“Identical” or “identity” as used herein in the context of two or more nucleic acids or polypeptide sequences, means that the sequences have a specified percentage of residues that are the same over a specified region. The percentage can be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of single sequence are included in the denominator but not the numerator of the calculation. When comparing DNA and RNA, thymine (T) and uracil (U) can be considered equivalent. Identity can be performed manually or by using a computer sequence algorithm such as BLAST or BLAST 2.0.

“Instructional material,” as that term is used herein, includes a publication, a recording, a diagram, or any other medium of expression which can be used to communicate the usefulness of the nucleic acid, peptide, and/or compound of the invention in the kit for identifying, diagnosing or alleviating or treating the various diseases or disorders recited herein. Optionally, or alternately, the instructional material may describe one or more methods of identifying, diagnosing or alleviating the diseases or disorders in a cell or a tissue of a subject. The instructional material of the kit may, for example, be affixed to a container that contains the nucleic acid, peptide, and/or compound of the invention or be shipped together with a container that contains the nucleic acid, peptide, and/or compound. Alternatively, the instructional material may be shipped separately from the container with the intention that the recipient uses the instructional material and the compound cooperatively.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

The terms “determining,” “detecting,” “measuring,” “assessing,” and “assaying” are used interchangeably and include both quantitative and qualitative measurement, and include determining if a characteristic, trait, marker, biomarker, or feature is present or not. Assessing may be relative or absolute. “Assessing the presence of′ includes determining the amount of something present, as well as determining whether it is present or absent.

The terms “patient,” “subject,” “individual,” and the like are used interchangeably herein, and refer to any animal, or cells thereof whether in vitro or in situ, amenable to the methods described herein. In certain non-limiting embodiments, the patient, subject or individual is a human.

A “therapeutic” treatment is a treatment administered to a subject who exhibits a sign or symptom of pathology, for the purpose of diminishing or eliminating that sign or symptom.

As used herein, the terms “therapy” or “therapeutic regimen” refer to those activities taken to alleviate or alter a disorder or disease state, e.g., a course of treatment intended to reduce or eliminate at least one sign or symptom of a disease or disorder using pharmacological, surgical, dietary and/or other techniques. A therapeutic regimen may include a prescribed dosage of one or more drugs or surgery. Therapies will most often be beneficial and reduce or eliminate at least one sign or symptom of the disorder or disease state, but in some instances the effect of a therapy will have non-desirable or side-effects. The effect of therapy will also be impacted by the physiological state of the subject, e.g., age, gender, genetics, weight, other disease conditions, etc.

The term “therapeutically effective amount” refers to the amount of the subject compound that will elicit the biological or medical response of a tissue, system, or subject that is being sought by the researcher, veterinarian, medical doctor or other clinician. The term “therapeutically effective amount” includes that amount of a compound that, when administered, is sufficient to prevent development of, or alleviate to some extent, one or more of the signs or symptoms of the disorder or disease being treated. The therapeutically effective amount will vary depending on the compound, the disease and its severity and the age, weight, etc., of the subject to be treated.

The terms “treat,” “treating,” and “treatment,” refer to therapeutic or preventative measures described herein. The methods of “treatment” employ administration to a subject, in need of such treatment, a method of the present invention, for example, a subject afflicted with a disease or disorder, or a subject who ultimately may acquire such a disease or disorder, in order to prevent, cure, delay, reduce the severity of, reduce the frequency of, or ameliorate at least one sign or symptom of the disease or disorder.

The term “variant,” as used herein, refers to a compound that differs from the compound of the present invention, but retains essential properties thereof. A non-limiting example of this is a polynucleotide or polypeptide compound having conservative substitutions with respect to the reference compound, commonly known as degenerate variants. Another non-limiting example of a variant is a compound that is structurally different, but retains the same active domain of the compounds of the present invention. Variants include N-terminal or C-terminal extensions, capped amino acids, modifications of reactive amino acid side chain functional groups, e.g., branching from lysine residues, pegylation, and/or truncations of a polypeptide compound. Generally, variants are overall closely similar, and in many regions, identical to the compounds of the present invention. Accordingly, the variants may contain alterations in the coding regions, non-coding regions, or both.

As used herein, the term “gel” refers to a three-dimensional polymeric structure that itself is insoluble in a particular liquid but which is capable of absorbing and retaining large quantities of the liquid to form a stable, often soft and pliable, but always to one degree or another shape-retentive, structure. When the liquid is water, the gel is referred to as a hydrogel. Unless expressly stated otherwise, the term “gel” will be used throughout this application to refer both to polymeric structures that have absorbed a liquid other than water and to polymeric structures that have absorbed water, it being readily apparent to those skilled in the art from the context whether the polymeric structure is simply a “gel” or a “hydrogel.”

As used herein, “scaffold” refers to a structure, comprising a biocompatible material that provides a surface suitable for adherence and proliferation of cells. A scaffold may further provide mechanical stability and support. A scaffold may be in a particular shape or form so as to influence or delimit a three-dimensional shape or form assumed by a population of proliferating cells. Such shapes or forms include, but are not limited to, films (e.g. a form with two-dimensions substantially greater than the third dimension), ribbons, cords, sheets, flat discs, cylinders, spheres, 3-dimensional amorphous shapes, etc.

The terms “biomarker” and “marker” are used herein interchangeably. They refer to a substance that is a distinctive indicator of a biological process, biological event and/or pathologic condition.

The term “body fluid” or “bodily fluid” as used herein refers to any fluid from the body of an animal. Examples of body fluids include, but are not limited to tear fluid, plasma, serum, and whole blood. A body fluid sample may be collected by any suitable method. The body fluid sample may be used immediately or may be stored for later use. Any suitable storage method known in the art may be used to store the body fluid sample. For example, the sample may be frozen at about-20 °C to about-80 °C.

“Biological sample” or “sample” as used herein means a biological material isolated from an individual. The biological sample may contain any biological material suitable for detecting the desired biomarkers, and may comprise cellular and/or non-cellular material obtained from the individual.

The term “control” or “comparator” describes a material comprising none, or a normal, low, or high level of one of more of the marker (or biomarker) expression products of one or more the markers (or biomarkers) of the invention, such that the comparator may serve as a control or reference standard against which a sample can be compared.

As used herein, the term “diagnosis” means detecting a disease or disorder or determining the stage or degree of a disease or disorder. Usually, a diagnosis of a disease or disorder is based on the evaluation of one or more factors and/or symptoms that are indicative of the disease. That is, a diagnosis can be made based on the presence, absence or amount of a factor which is indicative of presence or absence of the disease or disorder. Each factor, sign or symptom that is considered to be indicative for the diagnosis of a particular disease or disorder does not need be exclusively related to the particular disease; i.e. there may be differential diagnoses that can be inferred from a diagnostic factor, sign or symptom. Likewise, there may be instances where a factor, sign or symptom that is indicative of a particular disease or disorder is present in an individual that does not have the particular disease or disorder. The diagnostic methods may be used independently, or in combination with other diagnosing and/or staging methods known in the medical art for a particular disease or disorder.

As used herein, the phrase “difference of the level” refers to differences in the quantity of a particular marker, such as a nucleic acid (e.g., cryptic exon, etc.) or a protein, in a sample as compared to a control or reference level. For example, the quantity of a particular biomarker may be present at an elevated amount or at a decreased amount in samples of patients with a disease compared to a reference level. In one embodiment, a “difference of a level” may be a difference between the quantity of a particular biomarker present in a sample as compared to a control of at least about 1%, at least about 2%, at least about 3%, at least about 5%, at least about 10%, at least about 15%, at least about 20%, at least about 25%, at least about 30%, at least about 35%, at least about 40%, at least about 50%, at least about 60%, at least about 75%, at least about 80% or more. In one embodiment, a “difference of a level” may be a statistically significant difference between the quantity of a biomarker present in a sample as compared to a control. For example, a difference may be statistically significant if the measured level of the biomarker falls outside of about 1.0 standard deviations, about 1.5 standard deviations, about 2.0 standard deviations, or about 2.5 standard deviations of the mean of any control or reference group.

By the phrase “determining the level of marker (or biomarker) expression” is meant an assessment of the degree of expression of a marker in a sample at the nucleic acid or protein level, using technology available to the skilled artisan to detect a sufficient portion of any marker expression product.

“Differentially increased expression” or “up regulation” refers to expression levels which are at least 10% or more, for example, 20%, 30%, 40%, or 50%, 60%, 70%, 80%, 90% higher or more, and/or 1.1 fold, 1.2 fold, 1.4 fold, 1.6 fold, 1.8 fold, 2.0 fold higher or more, and any and all whole or partial increments there between compared to a comparator.

“Differentially decreased expression” or “down regulation” refers to expression levels which are at least 10% or more, for example, 20%, 30%, 40%, or 50%, 60%, 70%, 80%, 90% lower or less, and/or 2.0 fold, 1.8 fold, 1.6 fold, 1.4 fold, 1.2 fold, 1.1 fold or less lower, and any and all whole or partial increments there between compared to a comparator.

As used herein, a “primer” for amplification is an oligonucleotide that specifically anneals to a target or marker nucleotide sequence. The 3′ nucleotide of the primer should be identical to the target or marker sequence at a corresponding nucleotide position for optimal primer extension by a polymerase. As used herein, a “forward primer” is a primer that anneals to the anti-sense strand of double stranded DNA (dsDNA). A “reverse primer” anneals to the sense-strand of dsDNA.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

It is understood that embodiments described herein include “consisting” and/or “consisting essentially of” embodiments.

### DESCRIPTION

The present invention relates to human corneal organoids and methods of and methods of making compositions derived from any component of human corneal organoids. The present invention also provides methods of using human corneal organoids and/or human corneal organoid derived compositions. For example, in certain aspects the compositions described herein can be used for treatment of a wound in a subject. In some embodiments, the compositions described herein can be used to enhance or improve in vitro or ex vivo culturing of cells. The present invention also relates methods of sorting cells derived from human corneal organoids.

The present invention also relates to a method of diagnosing keratoconus (KC) or assessing the severity of KC in a subject based upon detection of the presence or relative level of a protein or RNA transcripts encoding a protein. The invention is based on the finding that the NRF2 antioxidant pathway is dysregulated in KC. In some embodiments, the method of diagnosing or assessing the severity of KC comprises measuring the level of an NRF2 target protein or RNA transcripts encoding an NRF2 target protein in a biological sample from a subject suspected of having, at risk of having, or at risk of developing KC. In some embodiments, the NRF2 target protein is selected from glutathione peroxidase 3 (GPX3) and malondialdehyde (MDA).

In some embodiments, the level of the NRF2 target protein or RNA transcripts encoding an NRF2 target protein in the biological sample from the subject is measured using one or more techniques selected from ELISA, immunofluorescence, and RT-PCR, including qRT-PCR. In some embodiments, the biological sample is selected from tear fluid, epithelial cells, serum, plasma, and blood.

In some embodiments, the method further comprises a step of administering a treatment or therapy to the subject that has been diagnosed with having, being at risk of having, or being at risk of developing KC to treat or prevent KC.

### Methods for Making Corneal Organoids

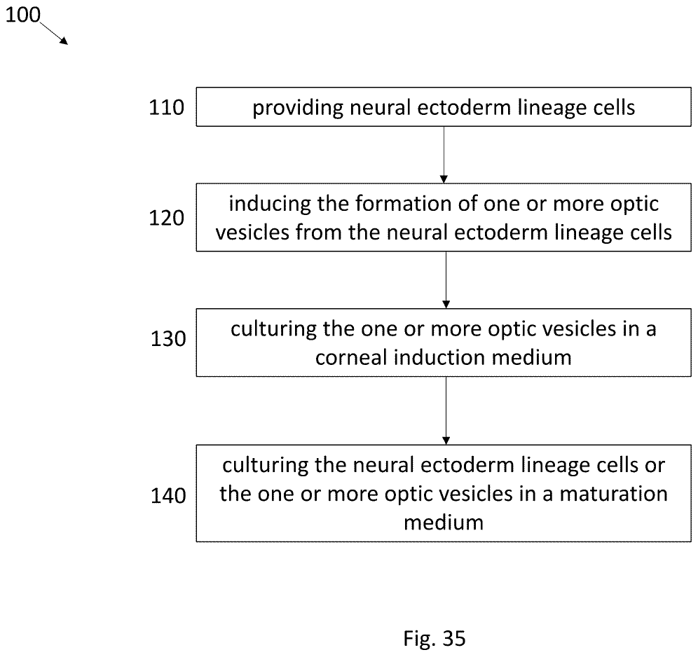

In one aspect, the present invention provides efficient methods for making one or more human corneal organoids. For example, as described herein, the presently described method for making human corneal organoids may increase the yield of human corneal organoids and/or generates human corneal organoids that have a high number of stromal cells. The presently described method for making human corneal organoids may also decrease batch variability among human corneal organoids. Now referring to FIG. 35, in some embodiments, the method comprises method 100 comprising the steps of 110 providing neural ectoderm lineage cells, 120 culturing the neural ectoderm lineage cells in an optic vesicle induction medium to induce the formation of one or more optic vesicles, and 130 culturing the one or more optic vesicles in a corneal induction medium. In some embodiments, the method further comprises 140 culturing the neural ectoderm lineage cells or the one or more optic vesicles in a maturation medium.

Neural ectoderm lineage cells of the method may be derived from any cell or by any method. In some embodiments, neural ectoderm lineage cells are derived from primary cells of a subject, induced pluripotent stem cells (iPSCs), embryonic stem cells, and/or somatic stem cells. The neural ectoderm lineage cells may be derived from primary cells that have been induced by any method to form induced pluripotent stem cells. iPSCs may be any iPSC line known in the art.

In some embodiments, neural ectoderm lineage cells of the method are derived from cell aggregates of induced pluripotent stem cells. Cell aggregates may comprise forced aggregates, spheroids, or any other 3D formation of cells. Cells of a cell aggregate may have primarily cell-cell contacts rather than cell-substrate or cell-surface contacts. Cell aggregates may be any size. For example, cell aggregates may be any dimension (i.e. diameter, volume, surface area, height, width). Cell aggregates may comprise any number of cells. In some embodiments, cell aggregates comprise or comprises about 2,000, 2,500, 3,000, 3,500, 4,000, 4,500, or 5,000 cells. In some embodiments, cell aggregates comprise in the range of 2,000 cells to 5,000 cells. In some embodiments, cell aggregates comprise in the range of about 2,000 cells to about 5,000 cells.

Cell aggregates may be formed by any method. In some embodiments, cell aggregates are formed by placing any number of cells in a tube, a well of a tissue culture plate, or any other container. The tube well, or other container may have a rounded bottom. In some embodiments, a rounded portion of the tube, well, or other container is configured such that it imparts a desired shape or curvature to a cell aggregate. The container may be substantially non-adherent to cells. In some embodiments, the cell aggregates are formed by centrifugation of the tissue culture plate that comprises a well containing cells, the tube containing cells, or any other container for the cell. In some embodiments, the cell aggregates are formed in a well of a 96 well plate. Centrifugation may be performed at any speed and for any amount of time. Centrifugation speed may relate to the compactness of the cell aggregate, for example faster centrifugation speeds may result in a more compact cell aggregate. In some embodiments, cells are centrifuged gently. In some embodiments, cells are centrifuged at a speed in the range between or between about 200 g to 500 g.

Cell aggregates of iPSCs may be formed in the presence of any media. The media may be suitable for the culture of iPSCs, for example mTesR™ media. In some embodiments, the media may comprise an agent that promotes the formation of cell aggregates or promotes cell viability. An agent may comprise any agent that decreases cell contractility and/or decreases apoptosis, for example blebbistatin or a ROCK inhibitor. Cell aggregates may be formed in a hypoxic environment. A hypoxic environment may promote cell survival and cell aggregation. In some embodiments, cell aggregates are cultured overnight or approximately 16 hours before they are induced to differentiate to neural ectoderm lineage cells.

Neural ectoderm lineage cells may be formed from iPSC cell aggregates. Exemplary methods for the induction of neural ectoderm lineage cells are described in Chandrasekaran et al., Stem Cell Res. 2017 December: 25:139-151 which is hereby incorporated by reference in its entirety. iPSC cell aggregates may be transferred to a normoxic environment for induction to neural ectoderm lineage cells. iPSC cell aggregates may be induced to differentiate to neural ectoderm lineage cells by culturing in a neural induction medium (NIM). NIM may comprise a suitable cell culture medium. The suitable cell culture medium may comprise Dulbecco's Modified Eagle Medium (DMEM), high glucose DMEM, and/or DMEM: F12 medium. NIM may comprise any additives including B-27™ supplement, B-27™ supplement without vitamin A, pyruvate, non-essential amino acids, GlutaMAX™ any salts, any buffers, and/or Essential-6 media supplement. In some embodiments, N-2 media supplement is substituted for Essential-6 media supplement. Essential-6 media supplement may comprise water, any buffers, any salts, insulin, holo-transferrin, L-ascorbic acid, and/or sodium selenite. Essential-6 media supplement may comprise NaHCO₃. In some embodiments, Essential-6 media supplement comprises or comprises about 0.97 g/L NaHCO₃, 0.97 g/L insulin, 0.535 g/L holo-transferrin, 3.2 g/L L-ascorbic acid, and/or 700 μg/L sodium selenite.

NIM may comprise or comprise about 1% B-27™ supplement without vitamin A. NIM may comprise or comprise about 1 mM pyruvate. NIM may comprise or comprise approximately 1× GlutaMAX™ or about 2 mM L-analyl-L-glutamine. NIM may comprise 1× Gibco™ MEM Non-Essential Amino Acids. NIM may comprise 0.88 g/L NaCl. NIM may comprise or comprise about 4% Essential-6 supplement. NIM may comprise or comprise about 38.8 mg/L NaHCO₃. NIM may comprise or comprise approximately 38.8 mg/L insulin or approximately 5 μg/mL insulin. NIM may comprise or comprise about 21.4 mg/L holo-transferrin. NIM may comprise or comprise about 128 mg/L L-ascorbic acid. NIM may comprise or comprise about 28 μg/L sodium selenite. Nim may comprise or comprise about 100 μM β-mercaptoethanol. Nim may comprise or comprise about 5 ng/ml basic fibroblast growth factor (bFGF). In some embodiments, osmolarity of NIM may be in the range of 330 mOsm to 340 mOsm. In some embodiments, the osmolarity of NIM is adjusted by the addition of NaCL to NIM. In some embodiments, the osmolarity of NIM is raised by 30 mOsm by the addition of NaCL to NIM. For example, 0.88 g/L NaCL may be added to NIM.

In some embodiments, extracellular matrix proteins are added to NIM. In some embodiments, the extracellular matrix proteins comprise Matrigel®. In some embodiments, the extracellular matrix proteins comprise fibronectin, recombinant fibronectin, laminin, and/or recombinant laminin. In some embodiments, the extracellular matrix proteins are included in a synthetic hydrogel. In some embodiments, NIM does not comprise extracellular matrix proteins. In some embodiments, NIM does not comprise Matrigel® (Martins-Costa C, et al., EMBO J. 2023 Nov. 15;42 (22): e113213). In some embodiments, a Wnt/beta-catenin inhibitor is added to NIM. In some embodiments, NIM does not comprise a Wnt/beta-catenin inhibitor. In some examples, iPSC cell aggregates are cultured in NIM in which Matrigel® and a Wnt/beta-catenin inhibitor is added. In some embodiments, the Wnt/beta-catenin inhibitor comprises IWR1 or IWR1-e. In some embodiments, Matrigel® is added to NIM such that the NIM comprises or comprises about 0.1%, 0.5%, 1%, 2%, 3%, 4%, or 5% Matrigel®. In some embodiments, Matrigel® is added to NIM such that the NIM comprises in the range of 0.1% to 5% Matrigel®. In some embodiments, IWR1-e is added to NIM such that the NIM comprises or comprises about 6 μM IWR1-e or 3 μM IWR1-e. In some embodiments, IWR1-e is added to NIM such that the NIM comprises or comprises about 0.5 μM IWR1-e, 1 μM IWR1-e, 2 μM IWR1-e, 3 μM IWR1-e, 4 μM IWR1-e, 5 μM IWR1-e, 6 μM IWR1-e, 7 μM IWR1-e, 8 μM IWR1-e, 9 μM IWR1-e, or 10 μM IWR1-e. In some embodiments, IWR1-e is added to NIM such that the NIM comprises in the range of 0.5 μM IWR1-e to 10 μM IWR1-e. In some embodiments, IWR1 is added to NIM such that the NIM comprises or comprises about 0.5 μM IWR1, 1 μM IWR1, 2 μM IWR1, 3 μM IWR1, 4 μM IWR1, 5 μM IWR1, 6 μM IWR1, 7 μM IWR1, 8 μM IWR1, 9 μM IWR1, or 10 μM IWR1. In some embodiments, IWR1 is added to NIM such that the NIM comprises in the range of 0.5 μM IWR1 to 10 μM IWR1.

For example, iPSC cell aggregates may be induced to differentiate to neural ectoderm lineage cells by exposing the iPSC cell aggregates to NIM that comprises extracellular matrix proteins and a Wnt/beta-catenin inhibitor. In some embodiments, iPSC cell aggregates are first incubated in NIM that comprises a first concentration of extracellular matrix proteins and a first concentration of a Wnt/beta-catenin inhibitor, secondly incubated in NIM that comprises a second concentration of extracellular matrix proteins and a second concentration of a Wnt/beta-catenin inhibitor, and thirdly incubated in NIM that comprises a third concentration of extracellular matrix proteins and a third concentration of Wnt/beta-catenin inhibitor. In some embodiments, the first concentration of extracellular matrix proteins comprises 2% Matrigel®. In some embodiments, the first concentration of extracellular matrix proteins comprises or comprises about 0%, 0.1%, 0.5%, 1%, 2%, 3%, 4%, or 5% Matrigel®. In some embodiments, the first concentration of extracellular matrix proteins comprises in the range of 0% to 5% Matrigel®. In some embodiments, the first concentration of a Wnt/beta-catenin inhibitor comprises or comprises about 0 μM IWR1, 0.5 μM IWR1, 1 μM IWR1, 2 μM IWR1, 3 μM IWR1, 4 μM IWR1, 5 μM IWR1, 6 μM IWR1, 7 μM IWR1, 8 μM IWR1, 9 μM IWR1, or 10 μM IWR1. In some embodiments, the first concentration of a Wnt/beta-catenin inhibitor comprises in the range of 0 μM IWR1 to 10 μM IWR1. In some embodiments, the first concentration of a Wnt/beta-catenin inhibitor comprises or comprises about 0 μM IWR1-e, 0.5 μM IWR1-e, 1 μM IWR1-e, 2 μM IWR1-e, 3 μM IWR1-e, 4 μM IWR1-e, 5 μM IWR1-e, 6 μM IWR1-e, 7 μM IWR1-e, 8 μM IWR1-e, 9 μM IWR1-e, or 10 μM IWR1-e. In some embodiments, the first concentration of a Wnt/beta-catenin inhibitor comprises in the range of 0 μM IWR1-e to 10 μM IWR1-e. The first incubation duration may be or may be about 1 day, 2 days, 3 days, 4 days, 5 days, 6 days, or 7 days. The first incubation duration may be in the range of 1 day to 7 days. In some embodiments, the second concentration of extracellular matrix proteins comprises or comprises about 0%, 0.1%, 0.5%, 1%, 2%, 3%, 4%, or 5% Matrigel®.

In some embodiments, the second concentration of extracellular matrix proteins comprises in the range of 0% to 5% Matrigel®. In some embodiments, the second concentration of a Wnt/beta-catenin inhibitor comprises 3 μM IWR1. In some embodiments, the second concentration of a Wnt/beta-catenin inhibitor comprises or comprises about 0 μM IWR1, 0.5 μM IWR1, 1 μM IWR1, 2 μM IWR1, 3 μM IWR1, 4 μM IWR1, 5 μM IWR1, 6 μM IWR1, 7 μM IWR1, 8 μM IWR1, 9 μM IWR1, or 10 μM IWR1. In some embodiments, the second concentration of a Wnt/beta-catenin inhibitor comprises in the range of 0 μM IWR1 to 10 μM IWR1. In some embodiments, the second concentration of a Wnt/beta-catenin inhibitor comprises or comprises about 0 μM IWR1-e, 0.5 μM IWR1-e, 1 μM IWR1-e, 2 μM IWR1-e, 3 μM IWR1-e, 4 μM IWR1-e, 5 μM IWR1-e, 6 μM IWR1-e, 7 μM IWR1-e, 8 μM IWR1-e, 9 μM IWR1-e, or 10 μM IWR1-e. In some embodiments, the second concentration of a Wnt/beta-catenin inhibitor comprises in the range of 0 μM IWR1-e to 10 μM IWR1-e. The second incubation duration may be or may be about 1 day, 2 days, 3 days, 4 days, 5 days, 6 days, 7 days, 8 days, 9 days, or 10 days. The second incubation duration may be in the range of 1 day to 10 days. In some embodiments, the third concentration of extracellular matrix proteins comprises or comprises about 0%, 0.1%, 0.5%, 1%, 2%, 3%, 4%, or 5% Matrigel®. In some embodiments, the third concentration of extracellular matrix proteins comprises in the range of 0% to 5% Matrigel®. In some embodiments, the third concentration of a Wnt/beta-catenin inhibitor comprises or comprises about 0 μM IWR1, 0.5 μM IWR1, 1 μM IWR1, 2 μM IWR1, 3 μM IWR1, 4 μM IWR1, 5 μM IWR1, 6 μM IWR1, 7 μM IWR1, 8 M IWR1, 9 μM IWR1, or 10 μM IWR1. In some embodiments, the third concentration of a Wnt/beta-catenin inhibitor comprises in the range of 0 μM IWR1 to 10 μM IWR1. In some embodiments, the third concentration of a Wnt/beta-catenin inhibitor comprises or comprises about 0 μM IWR1-e, 0.5 μM IWR1-e, 1 μM IWR1-e, 2 μM IWR1-e, 3 μM IWR1-e, 4 μM IWR1-e, 5 μM IWR1-e, 6 μM IWR1-e, 7 UM IWR1-e, 8 μM IWR1-e, 9 μM IWR1-e, or 10 μM IWR1-e. In some embodiments, the third concentration of a Wnt/beta-catenin inhibitor comprises in the range of 0 μM IWR1-e to 10 μM IWR1-e. The third incubation duration may be or may be about 1 day, 2 days, 3 days, 4 days, 5 days, 6 days, 7 days, 8 days, 9 days, 10 days, 11 days, 12 days, 13 days, 14 days, or 15 days. In some embodiments, the third incubation time is within the range of about 1 day to 15 days. In some embodiments, the third incubation time is within the range of 1 day to 15 days.

In some embodiments, the method comprises inducing the formation of one or more optic vesicles from the neural ectoderm lineage cells. In some embodiments, inducing the formation of one or more optic vesicles from the neural ectoderm lineage cells comprises incubating the neural ectoderm lineage cells in an optic vesicle induction medium. In some embodiments, the incubation may be for a duration of or of about 1 day, 2 days, 3 days, 4 days, 5 days, 6 days, 7 days, 8 days, 9 days, or 10 days. In some embodiments, the incubation may be for a duration in the range of 1 day to 10 days. In some embodiments, the neural ectoderm lineage cells comprise an aggregate before induction. In some embodiments, the neural ectoderm linage cell aggregates are placed in a low attachment plate before induction. The low attachment plate may have any size wells. In some embodiments, the low attachment plate is a 24 well plate. In some embodiments, individual organoids are separated from each other throughout the culture such that the organoids do not adhere to each other. For example, organoids in a culture may be maintained separate from each other while inducing the formation of one or more optic vesicles from neural ectoderm lineage cells, culturing the one or more optic vesicles in a corneal induction medium, and/or while incubating the culture with a maturation medium. For example, organoids in a culture may be maintained separate from each other beginning during the induction of the formation of one or more optic vesicles and for any number of days after, for example, 10 to 31 days.

In some embodiments, optic vesicle induction medium comprises NIM. In some embodiments, optic vesicle induction medium comprises an agent that activates the Sonic hedgehog pathway and/or activates Smoothened (SMO). In some embodiments, optic vesicle induction medium is configured to promote a desired level of Sonic Hedgehog (Shh) signaling. In some embodiments, a desired level of Shh signaling relates to a level of Shh signaling during native eye development or native early eye patterning. In some embodiments, a desired level of Shh signaling relates to a level of Shh signaling exhibited by corneal cells during native development. In some embodiments, optic vesicle induction medium comprises Smoothened Agonist (SMA). In some embodiments, optic vesicle induction medium does not comprise Smoothened Agonist (SMA). In some embodiments, optic vesicle induction medium comprises or comprises about 100 nM SMA, 90 nM SMA, 80 nM SMA, 70 nM SMA, 60 nM SMA, 50 nM SMA, 40 nM SMA, 30 nM SMA, 20 nM SMA, 10 nM SMA, 5 nM SMA, 4 nM SMA, 3 nM SMA, 2 nM SMA, 1 nM SMA, 0.5 nM SMA, or 0 nM SMA. In some embodiments the optic vesicle induction medium comprises less than or less than about 100 nM SMA, 90 nM SMA, 80 nM SMA, 70 nM SMA, 60 nM SMA, 50 nM SMA, 40 nM SMA, 30 nM SMA, 20 nM SMA, 10 nM SMA, 5 nM SMA, 4 nM SMA, 3 nM SMA, 2 nM SMA, InM SMA, or 0.5 nM SMA. In some embodiments the optic vesicle induction medium comprises in the range of 0 nM SMA to 100 nM SMA.

In some embodiments, the method comprises culturing the one or more optic vesicles in a corneal induction medium. In some embodiments, a cell culture that contains one or more optic vesicles in addition to any number of other cell types including neural ectoderm lineage cells are cultured in a corneal induction medium. The one or more optic vesicles may be cultured in a corneal induction medium for any duration including or including about 1 day, 2 days, 3 days, 4 days, 5 days, 6 days, 7 days, 8 days, 9 days, 10 days, 11 days, 12 days, 13 days, or 14 days. The one or more optic vesicles may be cultured in a corneal induction medium for a duration in the range of 1 day to 14 days.

In some embodiments, optic vesicles are separated from other cells in the culture before culturing the one or more optic vesicles in a corneal induction medium. Optic vesicles may be separated using any method. In some embodiments, the separation method comprises using a tungsten needle. For example, a tungsten needle may be used to sever the one or more optic vesicles from other cells in a culture. In some embodiments, a tungsten needle is moved back and forth across the cell culture or in an otherwise scissoring motion to sever the one or more optic vesicles from other cells in the cell culture. In some embodiments, gentle pipetting may be applied to the organoids to separate optic vesicles, lobes, or any other organoid components.

In some embodiments, corneal induction medium comprises long-term maturation medium (LTR). LTR may comprise any medium suitable for the culture of iPSCs or neural ectoderm lineage cells. In some embodiments, LTR comprises DMEM or DMEM: F12. In some embodiments, LTR comprises a DMEM: F12 in a 3:1 ratio or about a 3:1 ratio. In some embodiments, LTR comprises B-27™ supplement, pyruvate, non-essential amino acids, GlutaMAX™, taurine, any antibiotics, any antifungal agent, Penicillin, Streptomycin, any salts, and/or any buffers. In some embodiments, LTR comprises serum. In some embodiments, the serum is heat inactivated. In some embodiments, the serum comprises fetal bovine serum. In some embodiments, Knockout Serum Replacement (KSR) is used as a replacement for serum in LTR. For example, for any LTR formulations or compositions described herein that include serum, the serum may may be replaced by the same or similar concentration of KSR.

LTR may comprise or comprise about 1% B-27™ supplement. LTR may comprise or comprise about 1%, 2%, 3%, 4%, 5%, 6%, 7%, 8%, 9%, 10%, 11%, 12%, 13%, 14%, 15%, 16%, 17%, 18%, 19%, or 20% serum. LTR may comprise in the range of 1% to 20% serum. LTR may comprise or comprise approximately 1%, 2%, 3%, 4%, 5%, 6%, 7%, 8%, 9%, 10%, 11%, 12%, 13%, 14%, 15%, 16%, 17%, 18%, 19%, or 20% KSR. LTR may comprise in the range of 1% to 20% KSR. LTR may comprise or comprise about 1 mM pyruvate. LTR may comprise or comprise about 1× Gibco™ MEM Non-Essential Amino Acids. LTR may comprise or comprise about 1× GlutaMAX™. LTR may comprise or comprise about 2 mM L-analyl-L-glutamine. LTR may comprise or comprise about 1 mM taurine.

BMP-4 may be added to corneal induction medium that comprises KSR and/or does not comprise serum. BMP-4 may be added to corneal induction medium such that the concentration of BMP-4 in the corneal induction medium is or is about 0.1 nM, 0.5 nM, 1 nM, 1.5 nM, 2 nM, 2.5 nM, 3 nM. 3.5 nM, 4 nM, 4.5 nM, or 5 nM. BMP-4 may be added to corneal induction medium such that the concentration of BMP-4 in the corneal induction medium is in the range of 0.1 nM to 5 nM.

In some embodiments, corneal induction medium comprises an agent that activates the Sonic hedgehog pathway and/or activates Smoothened (SMO). In some embodiments, corneal induction medium is configured to promote a desired level of Sonic Hedgehog (Shh) signaling. In some embodiments, a desired level of Shh signaling relates to a level of Shh signaling during native eye development or native early eye patterning. In some embodiments, a desired level of Shh signaling relates to a level of Shh signaling exhibited by corneal cells during native development. In some embodiments, the corneal induction medium comprises Smoothened Agonist (SMA). In some embodiments, the corneal induction medium does not comprise Smoothened Agonist (SMA). In some embodiments, corneal induction medium comprises or comprises about 100 nM SMA, 90 nM SMA, 80 nM SMA, 70 nM SMA, 60 nM SMA, 50 nM SMA, 40 nM SMA, 30 nM SMA, 20 nM SMA, 10 nM SMA, 5 nM SMA, 4 nM SMA, 3 nM SMA, 2 nM SMA, 1 nM SMA, 0.5 nM SMA, or 0 nM SMA. In some embodiments, corneal induction medium comprises less than or less than about 100 nM SMA, 90 nM SMA, 80 nM SMA, 70 nM SMA, 60 nM SMA, 50 nM SMA, 40 nM SMA, 30 nM SMA, 20 nM SMA, 10 nM SMA, 5 nM SMA, 4 nM SMA, 3 nM SMA, 2 nM SMA, 1 nM SMA, or 0.5 nM SMA. In some embodiments corneal induction medium comprises in the range of 0 nM SMA to 100 nM SMA.

In some embodiments, after incubation with corneal induction medium, the one or more optic vesicles and/or any other cells in culture with the one or more optic vesicles are incubated with a maturation medium. Other cells in culture with the one or more optic vesicles may include any other neural ectoderm lineage cells.

Maturation medium may comprise LTR. For example, maturation medium may comprise compositions of LTR that comprise serum or KSR. Maturation medium may comprise all-trans-retinoic acid (ATRA). For example, maturation medium may comprise LTR to which ATRA has been added. Maturation medium may comprise or comprise about 50 nM, 100 nM, 200 nM, 300 nM, 400 nM, 500 nM, 600 nM, 700 nM, 800 nM, 900 nM, or 1 mM ATRA. Maturation medium may comprise in the range of 50 nM to 1 mM ATRA. The duration of incubation in maturation medium may be for up to 100 days or indefinitely. The duration of incubation in maturation medium may be or may be about 1 day, 2 days, 3 days, 4 days, 5 days, 10 days, 20 days, 30 days, 40 days, 50 days, 60 days, 70 days, 80 days, 90 days, 100 days, 110 days, 120 days, 130 days, 140 days, or 150 days. The duration of incubation in maturation medium may be in the range of 1 day to 150 days.

It should be appreciated that any incubations of the method with any medium may comprise any number of media changes and/or media replenishment at any frequency.

In some embodiments, corneal organoids are formed after any amount of time of incubation with maturation medium. In some embodiments, corneal organoids may be removed from the culture for any experimentation or may be used for any treatments. In some embodiments, any component of a corneal organoid, including cells or extracellular matrix components, may be extracted for downstream use in any application. Exemplary applications may include treatment of a subject and cell culture applications.

In some examples, the method comprises incubating and/or infecting the human corneal organoid or cells designated to form a human corneal organoid with a virus. Incubating organoids or cells with a virus may comprise infecting the organoids or cells with the virus. A human corneal organoid may be incubated with a virus at any timepoint of a method of making a human corneal organoid such as any methods of making organoids described herein. In some embodiments, a human corneal organoid is incubated with a virus at a timepoint in which the human corneal organoid has formed a corneal stromal layer and/or an epithelial layer. In some embodiments, a human corneal organoid is incubated with a virus at a timepoint in which the human corneal organoid has formed a corneal stromal layer and/or an epithelial layer that recapitulates a corneal stromal layer and/or an epithelial layer, respectively, of a human cornea at any point of human cornea development. In some embodiments, a human corneal organoid is incubated with a virus at any timepoint that is at least 30 days after the initiation of a method of making a human cornea organoid such as method 100. In some embodiments, a human corneal organoid is incubated with a virus while the human cornea organoid is positioned in maturation media of method 100. For example, a human cornea organoid may be incubated with a virus for any duration during step 140 of method 100 described herein.

Formed corneal organoids may be incubated with any virus, for example a virus configured to model any infection. In some examples, the formed corneal organoids are incubated with herpes simplex virus or a virus configured to model herpes simplex virus. Additional exemplary viruses for incubation with corneal organoids comprises herpes zoster virus, varicella zoster virus (VZV), SARS-COV-2, and any enterovirus. In some examples, the formed corneal organoids are incubated with In some embodiments, corneal organoids infected with herpes simplex virus are used in applications including drug screening or any drug discovery applications. For example, candidate drugs may be incubated with corneal organoids that have been previously incubated and/or infected by a virus. The organoids may then be analyzed to determine a candidate drug's effectiveness in treating disease.

Human corneal organoids may be derived by the methods of the invention from cells of any genetic background. For example, human corneal organoids of the invention may be derived any iPSC line or iPSCs derived from any subject. For example, human corneal organoids derived from iPSCs of a subject may be used in drug screens and/or any other experimentation useful in personalized medicine approaches. For example, human corneal organoids of the invention may be derived from iPSCs derived from a subject having any disease. For example, human corneal organoids of the invention may be derived from iPSCs derived from a subject having any disease, and the potential effectiveness of a candidate drug in treating a disease of the subject may be determined based on any experimentation on the organoid.

Human corneal organoids of the invention may be derived from cells with any genetic mutation or any single nucleotide polymorphisms (SNPs). In some embodiments, cells used to generate human corneal organoids may be engineered such that any number of genes are knocked out or such that any number of genes are overexpressed. Human corneal organoids generated from cells of different genetic backgrounds may be compared to draw conclusions about the effects of genetics on cornea development.

In some examples, the method comprises culturing the one or more HCOs in high-throughput. In some examples, a formed HCO is positioned in any number of separate wells of a tissue culture plate such as a 96 well plate. In some examples, cells designated to form an HCO are positioned in separate wells of a tissue culture plate, such as a 96 well plate, at any timepoint during a method of making an HCO. In some embodiments, high-throughput culture of organoids is useful for the testing of any number of treatments such as any number of drug candidates, any drug libraries, and/or any toxins.

### Compositions

In some aspects, the invention relates to compositions comprising human corneal organoids or a component of human corneal organoids. For example, in one embodiment, the composition comprises extracellular matrix (ECM) proteins extracted or isolated from human corneal organoids. In some examples, the composition comprises cells extracted or isolated from human corneal organoids. Compositions comprising extracellular matrix proteins and/or cells derived from human corneal organoids may be used to treat a subject and/or may be used in cell culture applications.

In some aspects, the invention relates to human corneal organoid-derived extracellular matrix (ECM). In some aspects, the invention relates to a composition comprising human corneal organoid-derived extracellular matrix (ECM). In some embodiments, human corneal organoids are decellularized such that ECM is left behind. Any methods for decellularization of tissue or organoids may be used. In some embodiments, human corneal organoids are decellularized such that the HCO-derived ECM is decellularized and retains the 3D structure of the organoid. Decellularization methods may comprise applying a detergent to a human corneal organoids. The detergent may be any detergent. In some embodiments, the detergent comprises a mild detergent, a neutral detergent, or a mild neutral detergent. Decellularization methods may comprise applying a non-detergent salt solution to a human corneal organoid. In some embodiments, ECM is extracted from human corneal organoids through the use of a lyophilization technique.

HCO-derived ECM may comprise any ECM proteins or other ECM components in any proportions or ratios. For example, HCO-derived ECM may comprise collagen I, collagen III, collagen V, fibronectin, lumican, biglycan, fibromodulin, decorin, glycosaminoglycans, keratan sulfate, and chondroitin sulfate. HCO-derived ECM may comprise any ECM proteins or other ECM components in proportions or ratios that are suggested by gene expression values in RNA sequencing data of human corneal organoids or the human cornea.

In one embodiment, the composition comprises a gel comprising HCO-derived ECM. The use of a gel may increase biocompatibility, support cell viability, and/or provide structural integrity for the effective delivery of HCO-derived ECM to a subject. For example, a gel may function as a supportive matrix or a carrier for HCO-derived ECM. In some embodiments, the gel comprises a hydrogel. Exemplary hydrogels include fibrin gel and PEGDA. Fibrin gel may comprise fibrin and thrombin an appropriate proportion for the formation of a gel. In some embodiments, the composition comprises any other pharmaceutical compositions for the treatment of the eye or treatment of the skin. In some embodiments, the composition comprises cells, growth factors, peptides, small molecules, antibiotics, or any other component useful for the treatment of the eye or treatment of the skin.

In some aspects, the invention provides cells derived from human corneal organoids. In some aspects, the invention relates to methods of using cells derived from human corneal organoids for treating a subject. For example, the cells may be applied to the eye of a subject or the skin of a subject. Human corneal organoids may be digested or dissociated by any method to obtain HCO cells or a solution comprising HCO cells. HCO cells may be sorted by any method to obtain a desired cell type, a desired ratio of cell types, or an enrichment of a desired cell type.

In some embodiments, the composition comprises HCO cells that have been sorted by the presence of any surface marker. Exemplary surface markers for sorting HCO cells include VEGFR1, CD-90/THY-1, and Podoplanin/PDPN. In some embodiments, the composition comprises VEGFR 1 positive cells, or VEGFR1 negative cells. VEGFR1 positive cells may be stromal or stromal-like cells, while VEGFR1 negative cells may be epithelial or epithelial-like cells. VEGFR1 positive, CD90 positive, and/or PDPN positive cells may be stromal or stromal-like cells. In some embodiments, cells are sorted such that cells positive for VEGFR1, CD90, and PDPN are isolated. In some embodiments, cells are sorted such that cells negative for VEGFR1, CD90, and PDPN are isolated. In some embodiments, the composition comprises epithelial or epithelial-like cells. In some embodiments, the composition comprises stromal or stromal-like cells. In some embodiments, the composition comprises a desired proportion or ratio of epithelial and stromal cells. In some embodiments, the composition comprises a desired proportion or ratio of stromal cell or stromal-like cell subtypes. Stromal cell subtypes may include keratocytes, progenitor keratocytes, differentiating keratocytes, keratocyte-like cells, keratocyte-like progenitor cells, proliferating keratocyte-like cells, immature keratocyte-like cells, fibroblasts, and myofibroblasts.

In some embodiments, the composition comprises HCO derived cells that have been expanded in tissue culture after being isolated from HCOs. In some embodiments, the composition comprises HCO derived cells that have been expanded in tissue culture after being sorted.

In one embodiment, the composition comprises a gel comprising HCO-derived cells. The use of a gel may increase biocompatibility, support cell viability, and/or provide structural integrity for the effective delivery of HCO-derived cells to a subject. For example, a gel may function as a supportive matrix or a carrier for HCO-derived cells. In some embodiments, the gel comprises a hydrogel. Exemplary hydrogels include fibrin gel and PEGDA. Fibrin gel may comprise fibrin and thrombin an appropriate proportion for the formation of a gel. In some embodiments, the composition comprises any other pharmaceutical compositions for the treatment of the eye or treatment of the skin. In some embodiments, the composition comprises cells, growth factors, peptides, small molecules, antibiotics, and/or any other component useful for the treatment of the eye or treatment of the skin.

In some embodiments, the invention provides a composition comprising HCO derived cells and HCO derived ECM. In some embodiments, the invention provides a composition comprising a gel comprising HCO derived cells and HCO derived ECM.

The present invention also provides pharmaceutical compositions comprising HCOs, HCO derived cells, and/or HCO derived ECM. Pharmaceutical compositions may be employed in admixtures with conventional excipients, i.e., pharmaceutically acceptable organic or inorganic carrier substances suitable for administration to the wound or treatment site. The pharmaceutical compositions may be sterilized and if desired mixed with auxiliary agents, e.g., lubricants, preservatives, stabilizers, wetting agents, emulsifiers, salts for influencing osmotic pressure buffers, coloring, and/or aromatic substances and the like. They may also be combined where desired with other active agents, e.g., other analgesic agents.

As used herein, “additional ingredients” include, but are not limited to, one or more of the following: excipients; surface active agents; dispersing agents; inert diluents; granulating and disintegrating agents; binding agents; lubricating agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” that may be included in the pharmaceutical compositions of the invention are known in the art and described, for example in Genaro, ed. (1985, Remington's Pharmaceutical Sciences, Mack Publishing Co., Easton, PA), which is incorporated herein by reference.

In some embodiments, compositions comprising components derived from human corneal organoids may be used to treat a subject. For example, ECM and/or cells derived from HCOs may be used to treat a subject. In some examples, a composition comprising a component of a human corneal organoid is configured to be applied to a subject. In some examples, the HCOs of a composition of the invention from which are derived from the cells of a subject. In some examples, the HCOs from which a component is extracted are derived from the cells of a subject.

### Methods of Sorting

In some aspects, the invention provides methods for sorting HCO derived cells or for the enrichment of HCO derived cell types. For example, as described herein, the method of sorting may provide rapid extraction of stromal enriched and epithelial enriched cell populations from human corneal organoids. In some embodiments, the invention provides a method of isolating a cell population enriched for stromal cells and/or a cell population enriched for epithelial cells from HCOs. In some embodiments, the invention provides a method of isolating a cell population that is depleted of stromal cells. In some embodiments, the method comprises the steps of digesting a human corneal organoid to obtain a cell suspension, contacting the cell suspension to a cell culture substrate that includes laminin, and collecting the cells that are not adhered to the cell culture substrate. The cells adhered to the cell culture substrate may be enriched for epithelial or epithelial-like cells, enriched for neural cells, enriched for corneal endothelial-like cells, enriched for melanocytes, and/or depleted of stromal or stromal-like cells. The cells that remain unadhered to the tissue culture substrate may be enriched for stromal or stromal-like cells, enriched for keratocyte-like cells, depleted of epithelial or epithelial-like cells, depleted of neural cells, depleted of melanocytes, and/or depleted of corneal endothelial-like cells.

In some embodiments, the cell culture substrate is coated with laminin. The laminin may comprise any laminin subtype. In some embodiments, the laminin comprises laminin 221, laminin 332, and/or laminin 511. In some embodiments, the laminin comprises iMatrix-221, iMatrix-332, and/or iMatrix-511. The cell culture substrate may comprise any tissue culture substrate known in the art. The cell culture substrate may comprise a dish, a flask, or a well of a tissue culture plate of any size.

In some embodiments, the HCO derived cell suspension is incubated in a laminin-coated tissue culture substrate for any duration of time to allow the adherence of at least some of the HCO derived cells. Longer incubation times may allow for more cells to adhere to the tissue culture substrate, therefore the duration of the incubation may relate to the cell types that adhere to the substrate. For example, the HCO derived cell suspension may be incubated in a laminin-coated tissue culture substrate for or for about 1 minute, 5 minutes, 10 minutes, 15 minutes, 20 minutes, 30 minutes, 40 minutes, 50 minutes, or 60 minutes. For example, the HCO derived cell suspension may be incubated in a laminin-coated tissue culture substrate for a duration in the range of 1 minute to 60 minutes. After the cell suspension is incubated the unadhered cells may be collected by any method.

### Methods of Culturing

In some aspects, the invention relates to methods of using human cornea organoid derived extracellular matrix (ECM) to promote the culture of cells. Human cornea organoid derived ECM may be beneficial for the culturing of difficult to culture cells for example primary cells from patients or any cells that are sensitive to xenogeneic materials. Human cornea organoid derived extracellular matrix may be used as a substitute for Matrigel® or any other basement membrane material used for culturing any cell types. For example, HCO-derived ECM may be used as a substitute for any xenogeneic materials. Human cornea organoid derived extracellular matrix may be made into the form of a gel. For example, a gel comprising HCO derived ECM may be used as a cell culture substrate for 2D and/or 3D cell cultures. Human cornea organoid derived extracellular matrix may be beneficial for human cell culturing in part due to the human origin of human cornea organoids.

HCO-derived ECM may promote certain differentiation outcomes of cells in culture. For example, HCO-derived ECM may promote the differentiation of certain cornea cell types.

In some embodiments, HCO derived ECM is applied to a tissue culture substrate for the subsequent culture of cells. For example, HCO derived ECM may be coated onto a tissue culture substrate. In some embodiments, HCO-derived ECM may be in the form of a 3D structure for example a scaffold. In some embodiments, cells are embedded in a gel comprising HCO-derived ECM. In some embodiments, HCOs are decellularized and the HCO derived ECM retains the 3D structure of the HCO or approximates the 3D structure of the HCO to form a scaffold. Therefore, cells may be seeded directly onto a decellularized HCO ECM that has maintained the 3D structure of an HCO. In some embodiments, HCO-derived ECM may be used as an additive to a cell culture medium. For example, HCO-derived ECM may be diluted in a cell culture medium at any concentration. In some embodiments, HCO-derived ECM may be used in conjunction with Matrigel® or any other basement membrane material used for culturing any cell types.

In some examples, contacting HCO derived ECM to a cell, a cell culture, or an organoid aids in the culture thereof. For example, contacting HCO derived ECM to a cell, a cell culture, or an organoid aids may aid in viability, proliferation, and/or desired cell type maintenance. In some examples, HCO derived ECM aids in the culture of primary cells from a subject or iPSC-derived cells. In some embodiments, the primary cells and/or iPSC-derived cells comprise corneal stromal cells, corneal epithelial stem cells, limbal stem cells, keratinocytes, and/or dermal fibroblasts. In some embodiments, cells are expanded in culture with the aid of HCO-derived ECM. In some embodiments, HCO derived ECM aids in the culture of organoids, for example HCOs. In some embodiments, HCO derived ECM aids in the culture of epithelial stem cells to study corneal epithelial defects or stem cell deficiencies. For example, autologous epithelial stem cells may be derived from cells with the genetic background of a subject and expanded in culture with the aid of HCO derived ECM.

### Methods of Treatment Via HCOs or Components Thereof.

In some aspects, the invention relates to a method of treating a subject. In some embodiments, the method comprises applying a composition of the invention to one or both eyes of a subject or to the skin of a subject. The composition may comprise any composition described herein. For example, the composition may comprise HCOs, isolated HCO derived cells, and/or isolated HCO derived ECM. Any method may be used to apply a composition of the invention to the eye or the skin.

In some examples, the subject has any disease or condition. In some examples, the subject has any disease or condition that pertains to the eye or the skin. In some examples, the subject has or is recovering from an eye injury or skin injury. In some examples, the eye or skin of the subject comprises a wound. In some embodiments, the subject has keratoconus, Fuchs endothelial dystrophy, infection-related corneal scarring, injury-related corneal scarring, a congenital corneal dystrophy, lattice corneal dystrophy, and/or Avellino corneal dystrophy.

In some examples, the composition is applied to the eye at any time after an eye wound. In some examples, the composition is applied to the skin at any time after a skin wound. For example, the composition may be applied to the eye shortly after the cornea of the eye has been injured or the composition may be applied to the eye after a scar has formed in response to a corneal injury. In some embodiments, the method comprises applying the composition to any number of times or for any frequency. For example, the composition may be applied hourly, twice daily, once daily, weekly, or monthly.

In some embodiments, the composition is applied to the eye in conjunction with any other pharmaceutical compositions for the treatment of the eye. In some embodiments, the composition may be applied with any other pharmaceutical composition to aid in the delivery of the composition to the eye. For example, the composition may be applied to the eye in conjunction with any cells, growth factors, peptides, small molecules, antibiotics, or any other component useful for the treatment of the eye.

In some embodiments, the composition is applied to the skin in conjunction with any other pharmaceutical compositions for the treatment of the skin. In some embodiments, the composition may be applied with any other pharmaceutical composition to aid in the delivery of the composition to the skin. For example, the composition may be applied to the skin in conjunction with any cells, growth factors, peptides, small molecules, antibiotics, or any other component useful for the treatment of the skin.

In some embodiments, the HCOs of a composition that is applied to the eye or skin of a subject is derived from the cells of the subject. In some embodiments, the cells of a composition that is applied to the or the skin of a subject are derived from HCOs that have been derived from cells of the subject. In some embodiments, the HCOs from which a component is derived that is then applied to the eye or skin of a subject is derived from the cells of the subject. For example, stem cells may be isolated or iPSCs may be derived from the cells of a subject and subsequently used to generate HCOs.

In some examples, a sealant is applied to the eye after the application of a composition of the invention to the eye. For example, a sealant may be applied to the eye after the application of a composition comprising HCOs, HCO derived cells, and/or HCO derived matrix to the eye. The sealant may be a fibrin sealant.

### Method of Diagnosis of KC

In some embodiments, the present invention provides methods for diagnosing a subject as having, being at risk of having, or being at risk of developing KC. In some embodiments, the invention provides methods for assessing the severity of KC in a subject.

In some embodiments, the method comprises detecting the presence or relative level of an NRF2 target protein or RNA transcripts encoding an NRF2 target protein in a biological sample from a subject. In some embodiments, the NRF2 target protein is selected from glutathione peroxidase (GPX) proteins and malondialdehyde (MDA). In some embodiments, the protein is GPX3.

The methods of detecting an NRF2 target protein (e.g., GPX3 or MDA) or RNA transcripts encoding an NRF2 target protein may be carried out using any assay or methodology known in the art. In various embodiments of the invention, methods of measuring an NRF2 target protein or RNA transcripts encoding an NRF2 target protein in a biological sample include, but are not limited to, PCR, including RT-PCR, RT-qPCR, and qPCR, sequencing, a gene chip or nucleic acid microarray assay, an immunochromatography assay, an immunodot assay, a Luminex assay, an ELISA assay, an ELISPOT assay, a protein microarray assay, a ligand-receptor binding assay, an immunostaining assay, a Western blot assay, a mass spectrophotometry assay, a radioimmunoassay (RIA), a radioimmunodiffusion assay, a liquid chromatography-tandem mass spectrometry assay, reverse phase protein microarray, a rocket immunoelectrophoresis assay, an immunohistostaining assay, an immunoprecipitation assay, a complement fixation assay, FACS, a protein chip assay, confocal imaging, live cell imaging, and fluorescence-based assays such as resazurin reduction assays for metabolic activity or viability, and caspase-3/7 activity assays for apoptosis detection (see also, 2007, Van Emon, Immunoassay and Other Bioanalytical Techniques, CRC Press; 2005, Wild, Immunoassay Handbook, Gulf Professional Publishing; 1996, Diamandis and Christopoulos, Immunoassay, Academic Press; 2005, Joos, Microarrays in Clinical Diagnosis, Humana Press; 2005, Hamdan and Righetti, Proteomics Today, John Wiley and Sons; 2007).

In some embodiments, the biological sample can be any sample from which a protein or nucleic acid molecule can be obtained. In some embodiments, the biological sample is selected from tear fluid, epithelial cells, serum, plasma, or blood. In some embodiments, epithelial cells are obtained from an epithelial scraping of the eye or tissue surrounding the eye. In some embodiments, the sample is tear fluid. Frequently the sample will be a “clinical sample” which is a sample derived from a patient.

A biological sample can be obtained by any suitable method. The biological sample can be used as the test sample; alternatively, the biological sample can be processed to enhance access to proteins, nucleic acids, or copies of nucleic acids, and the processed biological sample can then be used as the test sample. For example, in various embodiments, a protein or nucleic acid is prepared from a biological sample, for use in the methods.

In some embodiments, the method may comprise detecting the amount or level of a nucleic acid using one of a variety of nucleic acid detection assays. For example, traditional Northern blotting, nuclease protection, RT-PCR, qPCR, RT-qPCR, microarray, and differential display methods may be used for detecting gene expression levels. Methods for assaying for mRNA specifically include RT-PCR, RT-qPCR, Northern blots, slot blots, dot blots, and hybridization to an ordered array of oligonucleotides. Any method for specifically and quantitatively measuring a specific protein or mRNA or DNA product can be used.

Direct sequence analysis can also be used to detect a nucleic acid of interest. A sample comprising DNA or RNA can be used, and PCR or other appropriate methods can be used to amplify all or a fragment of the nucleic acid, and/or its flanking sequences, if desired. The nucleic acid, or a fragment thereof, is determined using standard methods.

In another embodiment, arrays of oligonucleotide probes that are complementary to target nucleic acid sequences can be used to detect and identify nucleic acids. For example, in one embodiment, an oligonucleotide array can be used. Oligonucleotide arrays typically comprise a plurality of different oligonucleotide probes that are coupled to a surface of a substrate in different known locations. These oligonucleotide arrays, also known as “Genechips,” have been generally described in the art, for example, U.S. Pat. No. 5,143,854 and PCT patent publication Nos. WO 90/15070 and 92/10092. These arrays can generally be produced using mechanical synthesis methods or light directed synthesis methods which incorporate a combination of photolithographic methods and solid phase oligonucleotide synthesis methods. See Fodor et al., 1991, Science 251, 767-777, Pirrung et al., U.S. Pat. No. 5,143,854 (see also PCT Application No. WO 90/15070) and Fodor et al., PCT Publication No. WO 92/10092 and U.S. Pat. No. 5,424,186. Techniques for the synthesis of these arrays using mechanical synthesis methods are described in, e.g., U.S. Pat. No. 5,384,261.

In some embodiments, the method uses an immunoassay detection system to measure the presence or amount of a protein in the sample. Exemplary immunoassays can include direct Enzyme-Linked Immunosorbent Assay (ELISA), indirect ELISA, sandwich ELISA, competitive ELISA, multiplex ELISA, lateral flow immunoassay, immunodiffusion assay, immunosensor assay, immunofluorescence assay, radioimmunoassay, ELISPOT technologies or mass spectrometry analysis methods.

In some embodiments, a sandwich ELISA is used. The sandwich ELISA measures the amount of a target between two layers of capture agents. The target to be measured must contain at least two binding sites, capable of binding to the capture agent, since at least two capture agents act in the sandwich. For this reason, sandwich assays are restricted to the quantitation of multivalent targets such as proteins or polysaccharides.

The Enzyme-Linked Immunosorbent Spot (ELISpot) assay employs the sandwich assay approach of the Enzyme-Linked ImmunoSorbent Assay (ELISA), with some variations. The capture agent is coated aseptically onto a polyvinylidene difluoride (PVDF)-backed microwell plate. The plate is blocked with serum proteins, cells of interest are plated out at varying densities, along with antigen or mitogen, and targets in a serum sample are captured locally by the coated antibody on the high surface area PVDF membrane. A second capture agent (biotinylated) reactive with a distinct epitope of the target is employed to detect the captured target. The detected target is then visualized using avidin-HRP, and a precipitating substrate (e.g. AEC). The colored end product (spot) represents an individual expressing the target antigen. The spots can be counted manually (e.g., with a dissecting microscope) or using an automated reader to capture the microwell images and to analyze spot number and size.

Lateral flow immunoassays, also called lateral flow tests, dipsticks or simply strip tests, are simple one- or two-step assays for the qualitative determination of targets directly in liquid samples. Specific lines, or zones, are “striped” onto, or applied to, a test membrane that contains capture agents designed to react with and bind to predefined targets of interest that may be present in a liquid test sample. When a liquid sample is applied to one end of the test membrane, the sample is drawn by capillary action along the longitudinal axis of the membrane strip. Targets of interest present in the sample interact with the capture agents, producing measurable and detectable changes along the striped agent assay test zones. The benefits of lateral flow tests include: (a) they have a user-friendly format; (b) a very short time is required to obtain the test result; (c) they have long-term stability over a wide range of climates; and (d) they are relatively inexpensive to make. These features make strip tests ideal for applications such as home testing, rapid point-of-care testing, and testing in the field for various environmental and agricultural analytes. In addition, they provide reliable testing that might not otherwise be available in developing countries.

A rapid lateral flow test generally comprises a system of overlapping porous materials containing the dried components needed to perform the test. These membranes are assembled in small strips, which can be placed into a plastic housing for ease in handling. Lateral flow tests can be used to detect any target that can be bound to a visually detectable capture agent attached to a solid support, both qualitatively and, in many cases, semi-quantitatively. A multiplexed lateral flow assay system can be used to detect multiple targets. A multiplexed lateral flow assay system aligns multiple lateral flow assays, or test strips, into a single large cassette. A liquid test sample is applied at a specific location and then divided and directed into multiple separate channels, with each channel containing capturing agents for detecting a specific target.

In various embodiments, the level of one or more of the NRF2 target protein (e.g., GPX3 or MDA) or RNA transcripts encoding an NRF2 target protein of the invention in the biological sample of the subject is compared with the level of a corresponding protein or RNA transcript in a comparator. Non-limiting examples of comparators include, but are not limited to, a negative control, a positive control, an expected normal background value of the subject, a historical normal background value of the subject, an expected normal background value of a population that the subject is a member of, or a historical normal background value of a population that the subject is a member of.

In some embodiments, the invention provides methods of diagnosing, monitoring the progression of, or treating KC by assessing the level of one or more of an NRF2 target protein or RNA transcript encoding an NRF2 target protein in a biological sample of the subject.

In some embodiments, the level of one or more NRF2 target protein or RNA transcripts encoding an NRF2 target protein of the invention is determined to be increased when the level of one or more NRF2 target protein or RNA transcripts encoding an NRF2 target protein detected in a biological sample of a subject is increased when compared with a comparator control.

In some embodiments, the level of one or more NRF2 target protein or RNA transcripts encoding an NRF2 target protein is determined to be increased when the level of the NRF2 target protein or RNA transcripts encoding an NRF2 target protein detected in a biological sample of a subject is increased by at least about 10%, at least about 20%, at least about 30%, at least about 40%, at least about 50%, at least about 60%, at least about 70%, at least about 80%, at least about 90%, at least about 100%, at least about 110%, at least about 120%, at least about 130%, at least about 140%, at least about 150%, at least about 160%, at least about 170%, at least about 180%, at least about 190%, at least about 200%, at least about 220%, at least about 240%, at least about 260%, at least about 280%, at least about 300%, at least about 350%, at least about 400%, at least about 450%, at least about 500%, at least about 550%, at least about 600%, at least about 650%, at least about 700%, at least about 750%, at least about 800%, at least about 850%, at least about 900%, at least about 950%, at least about 1000%, at least about 1100%, at least about 1200%, at least about 1300%, at least about 1400%, at least about 1500%, at least about 1600%, at least about 1700%, at least about 1800%, at least about 1900%, at least about 2000%, at least about 3000%, at least about 4000%, or at least about 5000%, when compared to with a comparator control.

In various embodiments of the assays of the invention, the level of the NRF2 target protein or RNA transcripts encoding an NRF2 target protein is determined to be increased when the level of the NRF2 target protein or RNA transcripts encoding an NRF2 target protein in the sample is increased by at least 1.2 fold, at least 1.3 fold, at least 1.4 fold, at least 1.5 fold, at least 1.6 fold, at least 1.7 fold, at least 1.8 fold, at least 1.9 fold, at least 2.0 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, at least 3.0 fold, at least 3.5 fold, at least 4.0 fold, at least 4.5 fold, at least 5.0 fold, at least 5.5 fold, at least 6 fold, at least 6.5 fold, at least 7 fold, at least 7.5 fold, at least 8 fold, at least 8.5 fold, at least 9 fold, at least 9.5 fold, at least 10 fold, at least 11 fold, at least 12 fold, at least 13 fold, at least 14 fold, at least 15 fold, at least 20 fold, at least 25 fold, at least 30 fold, at least 40 fold, at least 50 fold, at least 75 fold, at least 100 fold, at least 200 fold, at least 250 fold, at least 500 fold, or at least 1000 fold, when compared with a comparator control.

In some embodiments, the level of one or more NRF2 target protein or RNA transcripts encoding an NRF2 target protein of the invention is determined to be decreased when the level of one or more NRF2 target protein or RNA transcripts encoding an NRF2 target protein detected in a biological sample of a subject is decreased when compared with a comparator control.

In various embodiments of the assays of the invention, the level of NRF2 target protein or RNA transcripts encoding an NRF2 target protein is determined to be decreased when the level of the NRF2 target protein or RNA transcripts encoding an NRF2 target protein in the sample is decreased by at least about 10%, at least about 20%, at least about 30%, at least about 40%, at least about 50%, at least about 60%, at least about 70%, at least about 80%, at least about 90%, at least about 100%, at least about 110%, at least about 120%, at least about 130%, at least about 140%, at least about 150%, at least about 160%, at least about 170%, at least about 180%, at least about 190%, at least about 200%, at least about 220%, at least about 240%, at least about 260%, at least about 280%, at least about 300%, at least about 350%, at least about 400%, at least about 450%, at least about 500%, at least about 550%, at least about 600%, at least about 650%, at least about 700%, at least about 750%, at least about 800%, at least about 850%, at least about 900%, at least about 950%, at least about 1000%, at least about 1100%, at least about 1200%, at least about 1300%, at least about 1400%, at least about 1500%, at least about 1600%, at least about 1700%, at least about 1800%, at least about 1900%, at least about 2000%, at least about 3000%, at least about 4000%, or at least about 5000%, when compared with a comparator control.

In various embodiments of the method of the invention, the level of the NRF2 target protein or RNA transcripts encoding an NRF2 target protein is determined to be decreased when the level of the NRF2 target protein or RNA transcripts encoding an NRF2 target protein in the sample is decreased by at least 1.2 fold, at least 1.3 fold, at least 1.4 fold, at least 1.5 fold, at least 1.6 fold, at least 1.7 fold, at least 1.8 fold, at least 1.9 fold, at least 2.0 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, at least 3.0 fold, at least 3.5 fold, at least 4.0 fold, at least 4.5 fold, at least 5.0 fold, at least 5.5 fold, at least 6 fold, at least 6.5 fold, at least 7 fold, at least 7.5 fold, at least 8 fold, at least 8.5 fold, at least 9 fold, at least 9.5 fold, at least 10 fold, at least 11 fold, at least 12 fold, at least 13 fold, at least 14 fold, at least 15 fold, at least 20 fold, at least 25 fold, at least 30 fold, at least 40 fold, at least 50 fold, at least 75 fold, at least 100 fold, at least 200 fold, at least 250 fold, at least 500 fold, or at least 1000 fold, when compared with a comparator control.

In some embodiments, the level of GPX3 protein or RNA transcripts encoding GPX3 is determined to be increased when the level of GPX3 protein or RNA transcripts encoding GPX3 detected in a biological sample of a subject is increased when compared with a comparator control.

In some embodiments, the level of GPX3 protein or RNA transcripts encoding GPX3 is determined to be increased when the level of GPX3 protein or RNA transcripts encoding GPX3 detected in a biological sample of a subject is increased by at least about 10%, at least about 20%, at least about 30%, at least about 40%, at least about 50%, at least about 60%, at least about 70%, at least about 80%, at least about 90%, at least about 100%, at least about 110%, at least about 120%, at least about 130%, at least about 140%, at least about 150%, at least about 160%, at least about 170%, at least about 180%, at least about 190%, at least about 200%, at least about 220%, at least about 240%, at least about 260%, at least about 280%, at least about 300%, at least about 350%, at least about 400%, at least about 450%, at least about 500%, at least about 550%, at least about 600%, at least about 650%, at least about 700%, at least about 750%, at least about 800%, at least about 850%, at least about 900%, at least about 950%, at least about 1000%, at least about 1100%, at least about 1200%, at least about 1300%, at least about 1400%, at least about 1500%, at least about 1600%, at least about 1700%, at least about 1800%, at least about 1900%, at least about 2000%, at least about 3000%, at least about 4000%, or at least about 5000%, when compared to with a comparator control.

In various embodiments of the assays of the invention, the level of the GPX3 protein or RNA transcripts encoding GPX3 is determined to be increased when the level of the GPX3 protein or RNA transcripts encoding GPX3 in the sample is increased by at least 1.2 fold, at least 1.3 fold, at least 1.4 fold, at least 1.5 fold, at least 1.6 fold, at least 1.7 fold, at least 1.8 fold, at least 1.9 fold, at least 2.0 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, at least 3.0 fold, at least 3.5 fold, at least 4.0 fold, at least 4.5 fold, at least 5.0 fold, at least 5.5 fold, at least 6 fold, at least 6.5 fold, at least 7 fold, at least 7.5 fold, at least 8 fold, at least 8.5 fold, at least 9 fold, at least 9.5 fold, at least 10 fold, at least 11 fold, at least 12 fold, at least 13 fold, at least 14 fold, at least 15 fold, at least 20 fold, at least 25 fold, at least 30 fold, at least 40 fold, at least 50 fold, at least 75 fold, at least 100 fold, at least 200 fold, at least 250 fold, at least 500 fold, or at least 1000 fold, when compared with a comparator control.

In some embodiments, the level of GPX3 protein or RNA transcripts encoding GPX3 is determined to be decreased when the level of GPX3 protein or RNA transcripts encoding GPX3 detected in a biological sample of a subject is decreased when compared with a comparator control.

In various embodiments of the assays of the invention, the level of GPX3 protein or RNA transcripts encoding GPX3 is determined to be decreased when the level of GPX3 protein or RNA transcripts encoding GPX3 in the sample is decreased by at least about 10%, at least about 20%, at least about 30%, at least about 40%, at least about 50%, at least about 60%, at least about 70%, at least about 80%, at least about 90%, at least about 100%, at least about 110%, at least about 120%, at least about 130%, at least about 140%, at least about 150%, at least about 160%, at least about 170%, at least about 180%, at least about 190%, at least about 200%, at least about 220%, at least about 240%, at least about 260%, at least about 280%, at least about 300%, at least about 350%, at least about 400%, at least about 450%, at least about 500%, at least about 550%, at least about 600%, at least about 650%, at least about 700%, at least about 750%, at least about 800%, at least about 850%, at least about 900%, at least about 950%, at least about 1000%, at least about 1100%, at least about 1200%, at least about 1300%, at least about 1400%, at least about 1500%, at least about 1600%, at least about 1700%, at least about 1800%, at least about 1900%, at least about 2000%, at least about 3000%, at least about 4000%, or at least about 5000%, when compared with a comparator control.

In various embodiments of the method of the invention, the level of GPX3 protein or RNA transcripts encoding GPX3 is determined to be decreased when the level of GPX3 protein or RNA transcripts encoding GPX3 in the sample is decreased by at least 1.2 fold, at least 1.3 fold, at least 1.4 fold, at least 1.5 fold, at least 1.6 fold, at least 1.7 fold, at least 1.8 fold, at least 1.9 fold, at least 2.0 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, at least 3.0 fold, at least 3.5 fold, at least 4.0 fold, at least 4.5 fold, at least 5.0 fold, at least 5.5 fold, at least 6 fold, at least 6.5 fold, at least 7 fold, at least 7.5 fold, at least 8 fold, at least 8.5 fold, at least 9 fold, at least 9.5 fold, at least 10 fold, at least 11 fold, at least 12 fold, at least 13 fold, at least 14 fold, at least 15 fold, at least 20 fold, at least 25 fold, at least 30 fold, at least 40 fold, at least 50 fold, at least 75 fold, at least 100 fold, at least 200 fold, at least 250 fold, at least 500 fold, or at least 1000 fold, when compared with a comparator control.

In some embodiments, the level of MDA protein or RNA transcripts encoding MDA is determined to be increased when the level of MDA protein or RNA transcripts encoding MDA detected in a biological sample of a subject is increased when compared with a comparator control.

In some embodiments, the level of MDA protein or RNA transcripts encoding MDA is determined to be increased when the level of MDA protein or RNA transcripts encoding MDA detected in a biological sample of a subject is increased by at least about 10%, at least about 20%, at least about 30%, at least about 40%, at least about 50%, at least about 60%, at least about 70%, at least about 80%, at least about 90%, at least about 100%, at least about 110%, at least about 120%, at least about 130%, at least about 140%, at least about 150%, at least about 160%, at least about 170%, at least about 180%, at least about 190%, at least about 200%, at least about 220%, at least about 240%, at least about 260%, at least about 280%, at least about 300%, at least about 350%, at least about 400%, at least about 450%, at least about 500%, at least about 550%, at least about 600%, at least about 650%, at least about 700%, at least about 750%, at least about 800%, at least about 850%, at least about 900%, at least about 950%, at least about 1000%, at least about 1100%, at least about 1200%, at least about 1300%, at least about 1400%, at least about 1500%, at least about 1600%, at least about 1700%, at least about 1800%, at least about 1900%, at least about 2000%, at least about 3000%, at least about 4000%, or at least about 5000%, when compared to with a comparator control.

In various embodiments of the assays of the invention, the level of the MDA protein or RNA transcripts encoding MDA is determined to be increased when the level of the MDA protein or RNA transcripts encoding MDA in the sample is increased by at least 1.2 fold, at least 1.3 fold, at least 1.4 fold, at least 1.5 fold, at least 1.6 fold, at least 1.7 fold, at least 1.8 fold, at least 1.9 fold, at least 2.0 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, at least 3.0 fold, at least 3.5 fold, at least 4.0 fold, at least 4.5 fold, at least 5.0 fold, at least 5.5 fold, at least 6 fold, at least 6.5 fold, at least 7 fold, at least 7.5 fold, at least 8 fold, at least 8.5 fold, at least 9 fold, at least 9.5 fold, at least 10 fold, at least 11 fold, at least 12 fold, at least 13 fold, at least 14 fold, at least 15 fold, at least 20 fold, at least 25 fold, at least 30 fold, at least 40 fold, at least 50 fold, at least 75 fold, at least 100 fold, at least 200 fold, at least 250 fold, at least 500 fold, or at least 1000 fold, when compared with a comparator control.

In some embodiments, the level of MDA protein or RNA transcripts encoding MDA is determined to be decreased when the level of MDA protein or RNA transcripts encoding MDA detected in a biological sample of a subject is decreased when compared with a comparator control.

In various embodiments of the assays of the invention, the level of MDA protein or RNA transcripts encoding MDA is determined to be decreased when the level of MDA protein or RNA transcripts encoding MDA in the sample is decreased by at least about 10%, at least about 20%, at least about 30%, at least about 40%, at least about 50%, at least about 60%, at least about 70%, at least about 80%, at least about 90%, at least about 100%, at least about 110%, at least about 120%, at least about 130%, at least about 140%, at least about 150%, at least about 160%, at least about 170%, at least about 180%, at least about 190%, at least about 200%, at least about 220%, at least about 240%, at least about 260%, at least about 280%, at least about 300%, at least about 350%, at least about 400%, at least about 450%, at least about 500%, at least about 550%, at least about 600%, at least about 650%, at least about 700%, at least about 750%, at least about 800%, at least about 850%, at least about 900%, at least about 950%, at least about 1000%, at least about 1100%, at least about 1200%, at least about 1300%, at least about 1400%, at least about 1500%, at least about 1600%, at least about 1700%, at least about 1800%, at least about 1900%, at least about 2000%, at least about 3000%, at least about 4000%, or at least about 5000%, when compared with a comparator control.

In various embodiments of the method of the invention, the level of MDA protein or RNA transcripts encoding MDA is determined to be decreased when the level of MDA protein or RNA transcripts encoding MDA in the sample is decreased by at least 1.2 fold, at least 1.3 fold, at least 1.4 fold, at least 1.5 fold, at least 1.6 fold, at least 1.7 fold, at least 1.8 fold, at least 1.9 fold, at least 2.0 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, at least 3.0 fold, at least 3.5 fold, at least 4.0 fold, at least 4.5 fold, at least 5.0 fold, at least 5.5 fold, at least 6 fold, at least 6.5 fold, at least 7 fold, at least 7.5 fold, at least 8 fold, at least 8.5 fold, at least 9 fold, at least 9.5 fold, at least 10 fold, at least 11 fold, at least 12 fold, at least 13 fold, at least 14 fold, at least 15 fold, at least 20 fold, at least 25 fold, at least 30 fold, at least 40 fold, at least 50 fold, at least 75 fold, at least 100 fold, at least 200 fold, at least 250 fold, at least 500 fold, or at least 1000 fold, when compared with a comparator control.

In some embodiments, the presence or amount of an NRF2 target protein (e.g., GPX3 or MDA) or RNA transcripts encoding an NRF2 target protein in a biological sample of a subject is indicative of KC, risk of KC, or severity of KC in the subject. In some embodiments, the detection of the presence or an increased amount of an NRF2 target protein or RNA transcripts encoding an NRF2 target protein, as compared to a control or comparator as provided herein, is used to diagnose the subject as having, or as at risk of developing, KC. In various embodiments, the detection of the presence or amount of an NRF2 target protein or RNA transcripts encoding an NRF2 target protein is used to assess the progression of KC, or to assess the efficacy of a treatment method.

In some embodiments, the method of the invention is a diagnostic assay for diagnosing KC, or predicting the likelihood of developing KC, by determining the absolute amount or relative abundance of an NRF2 target protein (e.g., GPX3 or MDA) or RNA transcripts encoding an NRF2 target protein in a biological sample derived from the subject. In some embodiments, the subject is diagnosed as having KC when an NRF2 target protein or RNA transcripts encoding an NRF2 target protein is determined to be present at an increased abundance, relative to a comparator.

In some embodiments, the method of the invention is used to determine the severity of KC in a subject. In some embodiments, the amount of an NRF2 target protein (e.g., GPX3 or MDA) or RNA transcripts encoding an NRF2 target protein is correlated with disease severity. Accordingly, in some embodiments, a subject is diagnosed with having a particular severity of KC depending on the abundance of an NRF2 target protein or RNA transcripts encoding an NRF2 target protein relative to a comparator.

Information obtained from the methods of the invention described herein can be used alone, or in combination with other information (e.g., disease status, disease history, vital signs, blood chemistry, etc.) from the subject or from the biological sample obtained from the subject.

Methods of Treatment, Prevention, or Reduction of at Least One Symptom Associated with KC

The present invention also provides a method of treating or preventing KC, or reducing at least one symptom associated with KC in a subject. In one embodiment, the method comprises treating a subject identified by the methods of the invention as having or being at increased risk of developing KC through detection of an NRF2 target protein (e.g., GPX3 or MDA) or RNA transcripts encoding an NRF2 target protein in a biological sample of the subject. In some embodiments, the treatment comprises administering an effective amount of a composition to, or performing a procedure on, the subject. In some embodiments, the NRF2 target protein is selected from glutathione peroxidase (GPX) proteins and malondialdehyde (MDA). In some embodiments, the protein is GPX3.

In some embodiments, treatment comprises the use of eyeglasses, soft contact lenses, rigid gas permeable lenses, and scleral lenses.

In some embodiments, treatment comprises performing a procedure on a subject. Exemplary procedures that can be performed on subjects identified as having or at increased risk of developing JC include corneal cross-linking, intacts (intracorneal rings), corneal transplant, topography-guided phototherapeutic keratectomy (PTK), and conductive keratoplasty.

In some embodiments, treatment comprises administering an effective amount of a composition comprising at least one agent to treat KC. In one embodiment, the composition comprises at least one agent to reduce at least one symptom associated with KC. Exemplary agents that can be administered to subjects identified as having or at increased risk of developing KC include agents that reduce oxidative stress.

In some embodiments, the treatment comprises administering an effective amount of a composition comprising an agent for increasing the expression or activity of NRF2 or an NRF2 target protein, or RNA transcripts encoding NRF2 or an NRF2 target protein. In some embodiments, the NRF2 target protein is HMOX1 or GPX3. An agent that increases the level or activity of a protein, or the level of RNA transcripts encoding the protein, include, but should not be construed as being limited to, a chemical compound, a protein, a peptide, a peptidomemetic, an antibody, a ribozyme, a small molecule chemical compound, a nucleic acid, a vector, an antisense nucleic acid molecule (e.g., siRNA, miRNA, etc.), or combinations thereof.

The compositions of the invention can be administered to a subject in need in a wide variety of ways. In various embodiments, the composition of the invention is administered intraocularly, orally, intraoperatively, intravenously, intravascularly, intramuscularly, subcutaneously, intracerebrally, intraperitoneally, or by other soft tissue injection. Any administration may be a single administration of a therapeutic composition or multiple administrations. Administrations may be to a single site or to more than one site in the subject being treated. Multiple administrations may occur essentially at the same time or separated in time.

Compositions of the present invention may be administered in a manner appropriate to the disease to be treated (or prevented). The quantity and frequency of administration will be determined by such factors as the condition of the subject, and the type and severity of the subject's disease, although appropriate dosages may be determined by clinical trials.

The precise amount of the compositions of the present invention to be administered can be determined by a physician with consideration of individual differences in age, weight, disease type, extent of disease, and condition of the patient (subject).

The composition can be incorporated into any formulation known in the art. For example, the composition may be incorporated into formulations suitable for ocular administration, or another route of administration. Suitable compositions include, but are not limited to, suspensions, solutions, syrups, creams, pastes, plasters, lotions, discs, gels, eye drops, drug loaded contact lenses and the like. It should be understood that the formulations and compositions that would be useful in the present invention are not limited to the particular formulations and compositions that are described herein.

Although the description of compositions provided herein are principally directed to compositions which are suitable for ethical administration to humans, it will be understood by the skilled artisan that such compositions are generally suitable for administration to animals of all sorts. Modification of compositions suitable for administration to humans in order to render the compositions suitable for administration to various animals is well understood, and the ordinarily skilled veterinary pharmacologist can design and perform such modification with merely ordinary, if any, experimentation. Subjects to which administration of the therapeutic compositions of the invention is contemplated include, but are not limited to, humans and other primates, mammals including commercially relevant mammals such as non-human primates, cattle, pigs, horses, sheep, cats, and dogs.

In the method of treatment, the treatment, including administration of a procedure or composition, may be for either “prophylactic” or “therapeutic” purpose. When provided prophylactically, the treatment of the present invention is provided in advance of any sign or symptom, although in particular embodiments the treatment is provided following the onset of at least one sign or symptom to prevent further signs or symptoms from developing or to prevent present signs or symptoms from becoming more severe. The prophylactic treatment serves to prevent or ameliorate subsequent signs or symptoms. When provided therapeutically, the treatment is provided at or after the onset of at least one sign or symptom. Thus, the present invention may be provided either prior to the anticipated exposure to a disease-causing agent or disease state or after the initiation of the disease or disorder.

Subjects identified as having an increased level of at least one NRF2 target protein (e.g., GPX3 or MDA) or RNA transcripts encoding an NRF2 target protein can optionally be selected to receive treatment regimens, such as administration of prophylactic or therapeutic compounds or treatments to prevent, treat or delay the onset of KC.

Identifying a subject before they develop KC enables the selection and initiation of various therapeutic interventions or treatment regimens in order to delay, reduce or prevent the development or severity of the disease or disorder. In certain instances, monitoring the levels of at least one NRF2 target protein or RNA transcripts encoding an NRF2 target protein also allows for the course of treatment of the disease or disorder to be monitored. For example, a sample can be provided from a subject undergoing treatment regimens or therapeutic interventions (e.g., drug treatments, etc.) for KC. Samples can be obtained from the subject at various time points before, during, or after treatment.

The present invention also provides methods for identifying agents for treating KC that are appropriate or otherwise customized for a specific subject. In this regard, a test sample from a subject, exposed to a therapeutic agent, drug, or other treatment regimen, can be taken and the level of one or more NRF2 target protein or RNA transcripts encoding an NRF2 target protein can be determined. The level of one or more specific NRF2 target protein or RNA transcripts encoding an NRF2 target protein can be compared to in a sample derived from the subject before and after treatment, or can be compared across samples derived from one or more subjects who have shown improvements in risk factors as a result of such treatment or exposure.

### Kits

The present invention also pertains to kits useful in the methods of the invention. Such kits comprise various combinations of components useful in any of the methods described elsewhere herein, including for example, materials for quantitatively analyzing a protein or RNA transcript encoding a protein, and instructional material. For example, in one embodiment, the kit comprises components useful for the quantification of a desired nucleic acid in a biological sample. In another embodiment, the kit comprises components useful for the quantification of a desired polypeptide in a biological sample.

In a further embodiment, the kit comprises the components of an assay for diagnosing KC or assessing the severity of KC in a subject in need thereof, containing instructional material and the components for determining whether the level of a protein or RNA transcript of the invention in a biological sample obtained from the subject is elevated. In various embodiments, to determine whether the level of a protein or RNA transcript of the invention is elevated in a biological sample obtained from the subject, the level of the biomarker is compared with the level of at least one comparator control contained in the kit, such as a positive control, a negative control, a historical control, a historical norm, or the level of another reference molecule in the biological sample.

In a further embodiment, the kit comprises the components of an assay for monitoring the effectiveness of a treatment administered to a subject in need thereof, containing instructional material and the components for determining whether the level of a protein or RNA transcript of the invention in a biological sample obtained from the subject is modulated during or after administration of the treatment. In various embodiments, to determine whether the level of a protein or RNA transcript of the invention is modulated in a biological sample obtained from the subject, the level of the protein or RNA transcript is compared with the level of at least one comparator control contained in the kit, such as a positive control, a negative control, a historical control, a historical norm, or the level of another reference molecule in the biological sample. In certain embodiments, the ratio of the protein or RNA transcript and a reference molecule is determined to aid in the monitoring of the treatment.

### EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

### Example 1: Human Corneal Organoid Derived Decellularized Extracellular Matrix to Promote Scarless Corneal Wound Healing

Corneal disruption is a leading cause of blindness worldwide, yet limited donor cornea availability significantly restricts treatment options for keratoconus, Fuchs endothelial dystrophy, and infection- or injury-related corneal scarring. While epithelial and endothelial cell therapies have advanced, treatment of stromal disease or injuries remains a major challenge due to the lack of an ideal cell type or stromal biomaterial (R. Hayashi et al., Nature 531, 376-380 (2016), S. Kinoshita et al., N Engl J Med 378, 995-1003 (2018), M. Ueno et al., Am J Ophthalmol 237, 267-277 (2022)). To address this critical need, in this example, Human Corneal Organoids (HCOs) were developed from induced pluripotent stem cells (iPSCs). HCOs derived from iPSCs are an innovative system that mimics the regenerative potential of the fetal cornea and offers new avenues for corneal repair and stromal regeneration. The HCO stroma mimics an immature, developing matrix, a property that, in this example, was harnessed as a human-derived extracellular matrix (ECM) for tissue repair. Matrigel®, a basement membrane/ECM material of mouse origin is widely used in experimental settings, but its use in the clinic is limited (A. Passaniti, H. K. Kleinman, G. R. J Cell Commun Signal 16, 621-626 (2022)). The HCO-derived ECM of this example has wide-use in the clinic and experimental settings. Thus, the HCO-derived ECM of this example may be used for clinical wound-healing applications. In this example, HCOs and signaling networks underlying its interactions with stem cells are investigated in corneal repair, stromal integration, and anti-fibrotic remodeling such that HCO-derived ECM is positioned as a novel, translational, biomaterial for corneal regenerative medicine.

The decellularized HCO-derived ECM of this example mimics the regenerative properties of the fetal cornea. Therefore, decellularized HCO-derived is a unique biomaterial useful for wound healing and stromal repair. Unlike current scaffolds, which lack the dynamic remodeling features of native corneal tissue, the HCO ECM provides a structurally and biochemically relevant matrix that supports cell integration, tissue remodeling, and scarless healing. Decellularized HCO ECM is used in this example for corneal wound healing and as a filler for stromal injuries. Decellularized HCO ECM is a cutting-edge tissue-engineering strategy for regenerative treatments.

Additionally, 3D HCOs were developed from iPSCs, creating a scalable and reproducible in vitro model for mimicking key features of the developing human cornea. 3D HCOs developed from iPSCs represent a significant advancement over traditional models that rely on cadaveric donor tissue, which is limited in availability and variability. Unlike conventional systems, HCOs generate functional stromal cells within a physiologically relevant ECM, making HCOs a powerful too for studying corneal development and disease.

The efficacy of HCO-derived decellularized ECM as a novel biomaterial for corneal wound healing was evaluated. The 3D decellularized HCO matrix mimics the biological features of an immature regenerative corneal matrix that is expected to provided the niche for scarless wound healing. Scarless wound healing is critical for clear vision.

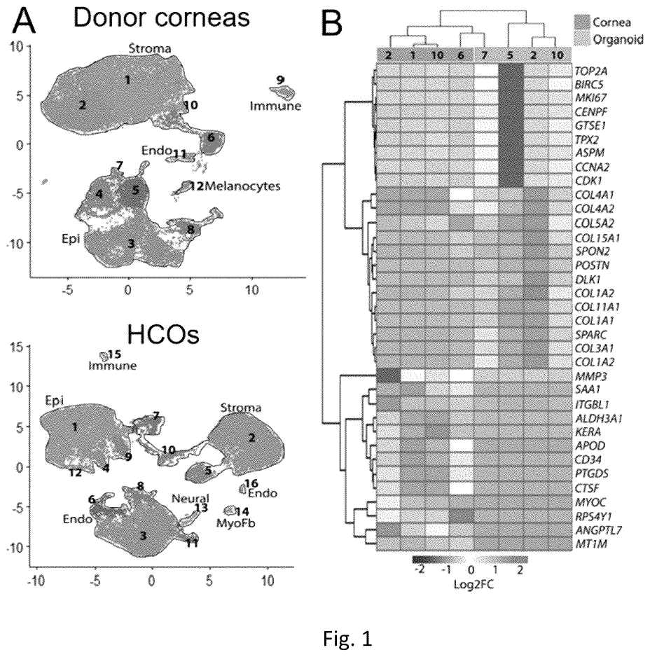

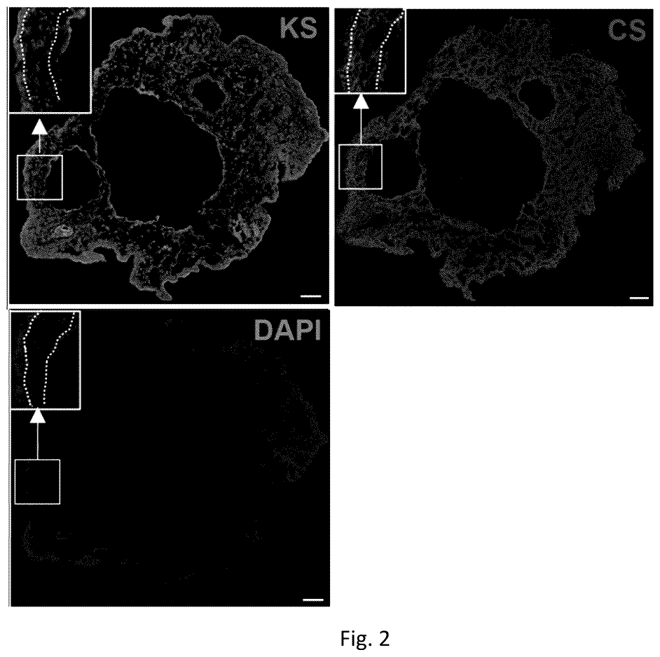

HCOs represent a breakthrough in corneal biology by co-differentiating stromal keratocytes, epithelial cells, and endothelial cells within a physiologically relevant 3D ECM (J. W. Foster et al., Cornea organoids from human induced pluripotent stem cells. Scientific reports 7, 41286 (2017), G. Maiti et al., PNAS Nexus 1, pgac246 (2022), S. Ashworth et al., Ocul Surf 35, 68-80 (2025)). Notably, these organoids recapitulate key features of the fetal cornea, which is known for its regenerative properties and resistance to fibrotic remodeling. The fetal corneal tissue has a unique ECM composition and microenvironment, and like all immature tissues, promote scarless healing. In contrast, the adult cornea has a highly organized ECM with minimal turnover, while injuries can lead to fibrotic repair and loss of vision (J. R. Hassell, D. E. Birk, Exp Eye Res 91, 326-335 (2010), R. C. de Oliveira, S. E. Wilson, Invest Ophthalmol Vis Sci 61, 28 (2020), S. E. Wilson, Exp Eye Res 207, 108594 (2021)). Single-cell RNA sequencing (scRNA-seq) and immunohistochemistry data revealed that HCO-derived stromal cells mirror developing corneal stroma, with cluster 2 (CL2) expressing collagens I, III, and V, while cluster 5 (CL5) and cluster 7 (CL7) show progenitor markers (FIG. 1). Additionally, unlike the adult cornea, HCOs deposit collagen III, fibronectin, and glycosaminoglycans (keratan sulfate (KS) and chondroitin sulfate (CS)), resembling regenerative or injured tissue (FIG. 2). Therefore, it is tested whether HCO-derived decellularized ECM resembling the fetal-like corneal stromal matrix, supports scarless wound healing without pathological ECM fibrosis. This is tested by evaluating the wound-healing properties of decellularized HCO ECM.

### Determination of Whether HCO-Derived Decellularized ECM Promotes Corneal Wound Healing.

The ability of HCO-derived ECM to integrate into corneal tissue and facilitate regenerative healing is assessed using a mouse model of corneal injury. Decellularized HCO ECM is introduced into mouse corneas injured with a scar-promoting debridement wound and scar-free healing is evaluated by in vivo slit-lamp microscopy. Tissue healing in harvested corneas is analyzed by immunohistochemistry, confocal microscopy, and electron microscopy.

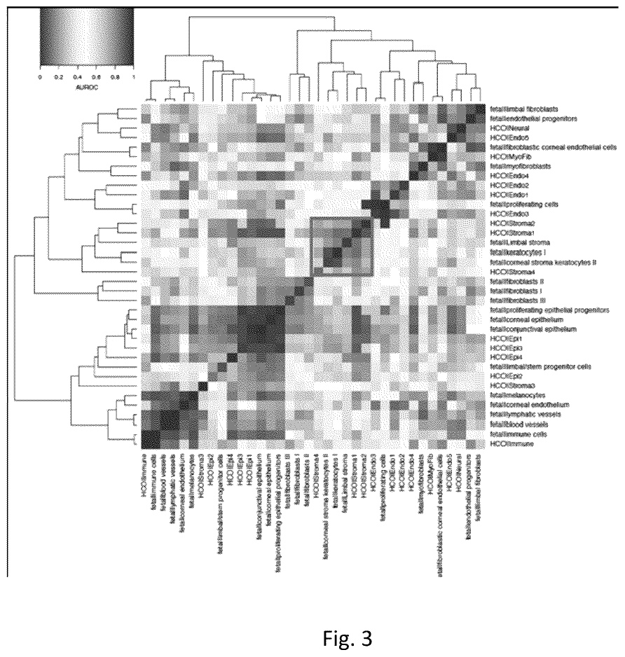

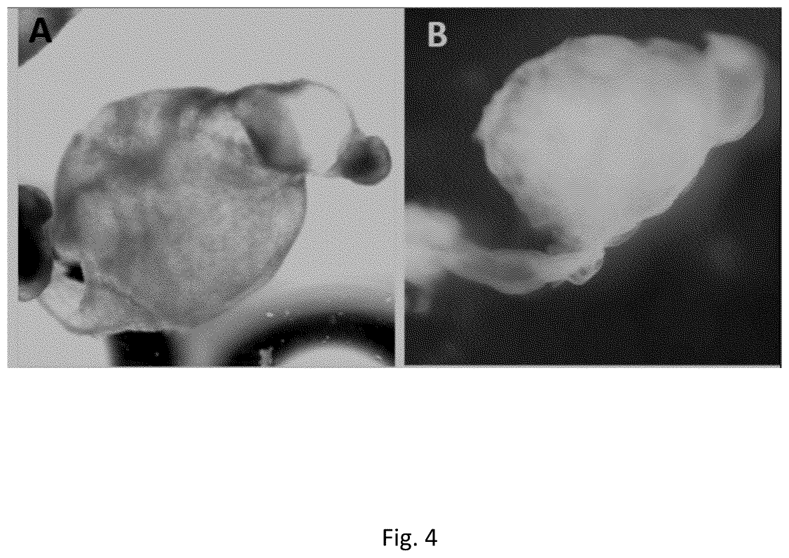

HCOs were shown to resemble the early 17-18-day human fetal cornea through scRNA-seq data (FIG. 3). HCOs were shown to have transcriptionally active ECM gene expression similar to wounded or immature cornea. Therefore, the question of whether the decellularized HCO matrix can promote scar-less wound healing is investigated. A decellularization procedure for HCOs was developed and experimental data shows that the decellularized HCO is devoid of cells but retains the ECM structure and components (FIG. 4).

Corneal wound healing, fibrosis, and inflammation are assessed after applying the decellularized HCO matrix. Specifically, a deep stromal injury is induced in one eye of a C57BL/6J mice using an Algerbrush® under anesthesia. HCO-ECM in fibrin gel or fibrin gel alone (control) is applied to the injury site. Wound healing and scar resolution is tracked using slit lamp biomicroscope (0-6 weeks), while corneal swelling is assessed via Optical Coherence Tomography (OCT). Corneal sections are immunostained for keratocyte markers (ALDH3A1, KERA), fibroblast marker (THY1), and myofibroblast markers (vimentin, desmin, a-SMA). Regenerating ECM components (collagen III, fibronectin) and fibrosis marker (SPARC) are evaluated. Fluorescently tagged collagen hybridizing peptides (CHP) are used to detect newly synthesized/denatured collagen. Corneal pro-inflammatory cytokines are analyzed using a multiplex assay. Transmission electron microscopy (TEM) is used to assess collagen fibril structure. This comprehensive approach provides insight into ECM remodeling, myofibroblast differentiation, and inflammatory responses during corneal wound healing.

### Investigation of the Regenerative Potential of Decellularized HCO Matrix.

Whether HCO-derived ECM provides a supportive microenvironment for corneal stem cell differentiation and ECM remodeling is evaluated. Corneal stem cells are seeded onto decellularized HCO ECM and differentiation into keratocytes, fibroblasts, and myofibroblasts is assessed using immunohistochemistry and flow cytometry. ECM-cellular interactions and anti-fibrotic signaling mechanisms by spatial transcriptomics and proteomics are determined.

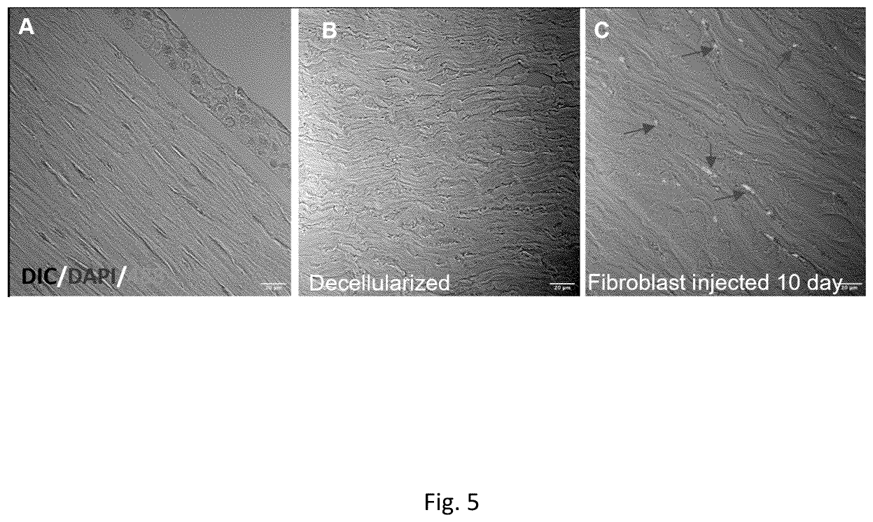

Dye-labeled primary stromal cells were introduced into the decellularized human cornea and were shown to have the capability of repopulating the matrix (FIG. 5). Based on this finding, whether corneal stem cells can differentiate into keratocytes once introduced into the decellularized HCO matrix is investigated.

Specifically, the question of whether HCO-derived ECM supports corneal/limbal stem cell survival and differentiation into keratocytes, fibroblasts, and myofibroblasts is assessed. Corneal stem/progenitor cells (e.g., ABCG2+, PAX6+, or ALDH3A1+, cells) are isolated from adult mice or human corneas by sorting. Cells are injected into decellularized HCO and are compared with standard substrates (e.g., Matrigel®). The decellularized HCO with the injected cells is then cultured for 7-14 days and differentiation is assessed using immunocytochemistry and flow cytometry for Keratocytes (KERA), fibroblasts (THY1, vimentin), and myofibroblasts (a-SMA).

How HCO-derived ECM influences cellular remodeling and ECM deposition by corneal cells is determined. Corneal fibroblasts or keratocytes are cultured on HCO-derived ECM and compared with adult corneal ECM. ECM remodeling is assessed using CHIP staining and immunostaining.

How HCO-derived ECM modulates signaling pathways that regulate fibrosis, inflammation, and regeneration is investigated. Spatial transcriptomics is used to compare gene expression profiles in corneal fibroblasts cultured on HCO-derived ECM vs. adult corneal ECM. Upregulated/downregulated pathways related to fibrosis (TGF-β, Wnt, PDGF) and regeneration (Notch, BMP, FGF) are identified. Proteomics and secretome analysis (LC-MS/MS) are used to identify ECM-derived bioactive factors and growth factors secreted by cells in response to HCO-derived ECM. Decellularized HCO-ECM is applied to wounded ex vivo human corneal tissue (organ culture model) and HCO ECM integration, cellular response, fibrosis markers are assessed.

### SUMMARY

HCO-derived ECM is demonstrated as a viable regenerative biomaterial to enhance corneal wound healing and reduce fibrosis in vivo. Molecular mechanisms underlying anti-fibrotic and pro-regenerative properties of HCO-derived ECM is identified. HCO-derived ECM's ability to support corneal stem cell differentiation and modulate ECM remodeling is validated. HCO-ECM may also be used as platform technology for applications beyond corneal healing. For example, HCO-ECM may be used to enhance graft integration and reduce rejection of corneal transplantation, as an alternative to amniotic membrane grafts in corneal ulcer treatment, to support limbal stem cell function in ocular surface disease therapy, or for skin wound healing.

Preclinical efficacy and biocompatibility of HCO-ECM in animal in vivo and human ex vivo models are validated. HCO-ECM is established as a safe, scalable biomaterial ready for clinical trials. Scalable and cost-efficient ECM production is achieved for clinical and commercial use. Early safety and efficacy data are generated to support large-scale clinical trials.

### Example 2: HCO-Derived Materials for Wound Healing and Regeneration

Corneal disruption is a major cause of blindness worldwide, and the limited availability of donor corneas significantly restricts transplantation options for diseases such as corneal-thinning keratoconus, Fuchs endothelial dystrophy and injury or infection-related corneal scarring. While epithelial and endothelial therapies have advanced, challenges remain in treating keratoconus, corneal scarring and dystrophy. To address the critical stromal need human cornea organoids (HCOs) were developed that mimic fetal cornea (Foster, J. W., et al., Sci Rep, 2017. 7: p.41286, Maiti, G., et al., PNAS Nexus, 2022. 1 (5): p. pgac246). In this example, the HCO's stromal cells and its immature extracellular matrix were investigated for their wound healing and regenerative potential.

Generated from pluripotent stem cells the HCOs present a breakthrough model system of co-differentiating stromal keratocyte, epithelial and endothelial cells in a more cornea-like 3-dimensional ECM environment. The TGF-β signaling network regulates cell differentiation, and the balance between healthy and fibrotic ECM synthesis (Massague, J. and D. Sheppard, Cell, 2023. 186 (19): p.4007-4037). Therefore, the immature cornea-like HCO was used to elucidate pre-regenerative TGF-β signals that can lead to scarless wound healing treatments of the cornea. The wound healing functions of HCO stromal ECM extract itself are also tested. Regenerative potentials of the HCO stromal cells were ascertained by functionally testing HCO stromal cells in human donor corneas and in vivo mouse models. The findings in this example elucidate therapeutically targetable TGF-β signaling intermediates and develop the HCO as a novel stroma source.

### Identification of Active TGF-β Signals in HCOs and Assessment of the HCO ECM as a Novel Wound Healing Biomaterial

It was hypothesized that TGF-β-mediated-fibrotic signals are restricted in HCOs compared to adult donor (DN) stromal cells and the HCO ECM promotes wound healing responses in cell culture. The expression of TGF-β network genes in HCOs was shown by single cell RNA sequence (scRNA-seq) data. This data showing the expression TGF-β network genes in HCOs enabled the querying of TGF-β fibrosis-limiting signals. HCOs are rich in epithelial and stromal cells, but are poor in immune cells. Therefore, HCOs were ideal for investigating the crosstalk between these major corneal cell types. The HCO stroma contains proteoglycans and fibrillar collagens, typical of immature corneas, which is a basis of the hypothesis that TGF-β-mediated-fibrotic signals are restricted in HCOs compared to adult donor (DN) stromal cells and the HCO ECM promotes wound healing responses in cell culture.

The TGF-β network in HCOs and DN spheroid cultures (as a control) are perturbed, and cellular responses are evaluated using scRNA-seq and phosphoproteome and ECM analyses. Wound healing functions of decellularized HCO ECMs are tested in cell culture and mouse models.

It is also tested whether TGF-β-mediated-fibrotic signals are highly regulated in HCOs and whether its ECM is supportive of scarless wound healing.

HCOs are challenged with TGF-β1 and TGF-β3. Signal transduction is then evaluated by immunoblotting and progenitor, keratocyte, fibroblast, and myofibroblast phenotypes are assessed by scRNA-seq. ECM changes are assayed by immunohistology, confocal and electron microscopy and proteomics. The decellularized HCO ECM extracts are assessed for wound healing responses in donor (DN) stromal cell culture assays.

The fetal quality of HCOs suggests built-in mechanisms that resist TGF-β-controlled fibrotic ECM production (Maiti, G., et al., PNAS Nexus, 2022. 1 (5): p. pgac246, Ashworth, S., et al., Ocul Surf, 2025. 35: p. 68-80). This regulation is explored by treating HCOs with TGF-β1 (TGF-β2 may elicit similar responses) or TGF-β3, or a TGFBR1 kinase inhibitor that blocks all SMA-dependent downstream signals and effects on cellular phenotypes and ECM production are determined. Corneal fibroblast spheroid cultures that have been studied previously are included as a control, because it has the 3D environment, but unlike HCOs contain differentiated corneal fibroblasts (Scott, S. G., et al., Exp Eye Res, 2011. 93 (6): p. 898-905). Like the immature corneal stroma, the HCO stroma may have regenerative, wound healing properties. Therefore, the effects of decellularized HCO-ECM on cell migration assays or scratch assays are tested. The HCO-preparations on mouse corneal wound healing are also tested. It is expected that the HCO ECM impacts the wound healing response which entails apoptosis, mesenchymal to epithelial transition, as well as formation of activated fibroblasts, myofibroblasts, and wound closure (Medeiros, C. S., et al., Invest Ophthalmol Vis Sci, 2018. 59 (10): p. 4044-4053, Wilson, S. E., et al., Matrix Biol, 2017. 64: p. 17-26).

The immature quality of the HCO ECM was shown. scRNA-seq showed high expression of collagen types I, III, and V in the large stromal cluster 2 (CL2) as was seen in developing corneal stroma, which cluster 5 (CL5) and cluster 7 (CL7) were more in transition, expressing proliferation and progenitor markers. By contrast, collagen transcripts were barely detectable in the adult resting cornea as there is very little ECM turnover. In addition to collagen V, the HCOs deposited a collagen III and a fibronectin matrix typical of developing or injured corneas, no collagen type III staining was detectable in the adult DN cornea. The HCOs also deposited the glycosaminoglycans chondroitin sulfate (CS) and keratan sulfate (KS) as in the developing cornea.

Culture conditions were significantly improved for purposes of high HCO yield and for assessing TGF-β response. Multiple conditions were tested using a stem cells line carrying two fluorescently tagged retinal genes. It was found that reducing hedgehog signaling by removing smoothened agonist during days 15 to 21 of development supported high cornea over retina organoids. Fetal bovine serum (contains TGF-β and other growth factors) was replaced with defined KnockOut™ serum replacement (KSR) media (ThermoFisher) and found unperturbed HCO growth. This enabled accurate assessments of TGF-β response.

HCOs express TGF-β network genes. HCOs were cultured in standard FBS media, which contains TGF-β, and not surprisingly express TGF-β network genes. The TGFBR1, TGFBR2 receptors, the R-SMADs and co-SMAD4 are expressed well in the stromal and epithelial clusters (CLs). The autoregulated ligands are also expressed. TGF-β1 is in the major differentiating stromal Cluster 2 (CL2) and the progenitor/proliferating Cluster 5 (CL5), TGF-β2 expression is weaker but similar in pattern, while TGF-β3 is more restricted to stromal CL2. THSB1, TSHD4 and ITGA5/ITGB, involved in releasing active TGF-ligand, are more specifically expressed in the stromal Cluster 7 (CL7), possibly a center of active signaling. It is examined how these signaling centers change in HCOs treated with the exogenous ligands or the TGFBR1 inhibitor. How the TGF-β-SMAD2/3 and the SMAD1/5/8 axes are activated in HCOs is also tracked. Activation/phosphorylation of SMAD1/5/8 within 30 minutes of TGF-β1 treatment of DN corneal stroma monolayer culture was noted (Foster, J., et al., PLOS One, 2014. 9 (9): p. e106556). Consequences of signals downstream of TGF-β1 and TGF-β3 on the two SMAD axes, and the noncanonical axis to impact keratocyte differentiation and ECM deposition are investigated.

Methods and Materials for experiments regarding the identification of active TGF-β signals in HCOs and assessment of the HCO ECM as a novel wound healing biomaterial are described.

### HCO Cultures

A robust pipeline for generating HCOs using three independent stem cell lines was developed. HCOs were developed from two independent line having similar timelines of growth and ECM staining (Ashworth, S., et al., Ocul Surf, 2025. 35: p. 68-80). TGF-β is tested and ECM is assayed from 3 independent HCO lines. All reagents are standardized and are endotoxin- and mycoplasma-free. FBS, a source of lot-dependent variability, was replaced with KnockOut™ serum replacement (KSR) media. All RNA-seq and proteomics have biological triplicates, and are quality-checked at each step.

### Primary Donor (DN) Corneal Stromal Cells

Keratocyte isolation was performed from DN corneas and 16 different low passage stocks were used (Scott, S. G., et al., Exp Eye Res, 2011. 93 (6): p. 898-905, Foster, J., et al., PLOS One, 2014. 9 (9): p. e106556, Foster, J. W., et al., Invest Ophthalmol Vis Sci, 2018. 59 (7): p. 2977-2986). These are used as controls as monolayer or spheroid cultures in this example as indicated. All antibodies used are standard and validated.

### Mouse Studies Used in all of Example 2

8-10-week-old male and female C57BL/6/J mice, randomized into groups of 6-10 animals, are used. Both sexes are used in each experiment. Clear sex-based differences give rise to the need for studying each group separately. All biomicroscopy and OCT methods are standardized. Scoring is blinded. Immunohistology is quantified using ImageJ.

### Statistical Methods

Two-sample t-test and one- or two-way ANOVA, Wilcoxon rank-sum test and Kruskal-Wallis ANOVA, and Pearson correlation are used as necessary for comparing two and multiple groups. All experiments are repeated 3-5 times. For control and treated mice unpaired Student's t test, and statistical significance set at ≤ 0.05 are used. For the injury models, sample sizes of 8 and 8 yield 80% power to detect mean difference of 1.51 standard deviations (SD) and a significance level of 0.05. For longitudinal biomicroscopy and OCT measurements, sample sizes of 5 and 5 achieve 80% power to detect a difference of 1.44 SD.

### HCO Treatments

HCOs grown in FBS-free KSR media are treated with TGF-β1 or TGF-β3 between 0.1 to 5 ng/m doses. 2 ng/mL TGF-β1 is used on human primary stromal cell monolayer culture (Foster, J., et al., PLOS One, 2014. 9 (9): p. e106556.). 0.1 ng/ml was used on primary cell cultures in 3D collagen constructs. HCOs are also treated with 10 μM TGFBR1 inhibitor, SB431542, to block all TGF-β-SMAD4-dpendent canonical signals (Oyanagi, J., et al., Exp Cell Res, 2014. 326 (2): p. 267-79).

### TGF-β Signal Assessment

Signal transduction is assayed by immunoblotting for serine/threonine phosphorylated SMAD2/3 and SMAD 1/5/8, both going through co-SMAD4, and the noncanonical axis is assayed by tyr-phosphorylated TGFBR2 and MAPK pathway components. Bulk RNA seq and phosphoproteome analyses are also performed on treated and untreated HCOs for signal transduction assessments. As additional controls, corneal fibroblast spheroid cultures are included. A TGF-β response panel is used to detect upregulation of partner transcription factors, signal intermediates and LTBPs that control signal strength (Rifkin). For selected ECM genes (COL3A1, COL1A1, FN1, LUM, DCN, SPARC, KERA) Taqman qRTPCR is used (Shao, H., et al., J Proteomics, 2011, Shinde, V., et al., Sci Rep, 2020. 10 (1): p. 9907).

### Cellular Phenotypes

scRNA-seq on treated HCOs is performed to elucidate how perturbation of the TGF-β network regulate cellular phenotypes. For morphological assessments, the HCOs are fixed, sectioned, and immunostained for keratocytes/fibroblasts and myofibroblasts using markers as listed. Flow cytometry is also used to analyze proportions of epithelial, endothelial, and stromal cells. Clonal expansion tests are performed on HCO-stromal cells as described for limbal stem and progenitor cell assessments (Basu, S., et al., 2014. 6 (266): p. 266ra172). The trajectory and pseudo time analysis predicted a pool of stromal precursor cells (DPP+, PIEZO2+) in the Clusters 3 (CL3) and Cluster 11 (CL11). Flow cytometry is also used to quantify these precursor progenitor-like cells (PIEZO2-Cat #83488-4-RR, Thermo and DPP-4 Cat #12-0269-42, eBioscience).

### ECM Structure

The ECM is immunostained for collagens I, III, and V, fibronectin, lumican, biglycan, decorin, keratocan, CS, and KS, and collagen deposition is assessed using picrosirius stain, Masson's trichrome (Scott, S. G., et al., Exp Eye Res, 2011. 93 (6): p. 898-905, Ashworth, S., et al., Ocul Surf, 2025. 35: p. 68-80, Engler, C., et al., Am J Ophthalmol, 2011. 151 (5): p. 752-759 e2, Foster, J. W., et al., Invest Ophthalmol Vis Sci, 2018. 59 (7): p. 2977-2986, Chakravarti, S., et al., Dev Dyn, 2006. 235 (9): p. 2493-506, Wu, F. and S. Chakravarti, J Immunol, 2007. 179 (10): p. 6988-7000). Denatured collagen is quantified using fluorescently tagged collagen hybridizing peptides (CHP, Advanced Biomatrix) (Yuan, Y., et al., Ocul Surf, 2025). TEM is also used for collagen fibril structure (Foster, J. W., et al., Sci Rep, 2017. 7: p. 41286, Chakravarti, S., et al., Invest Ophthalmol Vis Sci, 2003. 44 (6): p. 2422-32, Birk, D. and S. Chakravarti, Investigative Ophthalmology and Visual Sciences, 1999. 40: p. S179, Chen, S., et al., Dev Dyn, 2010. 239 (3): p. 844-54).

### Assessment of Decellularized HCO ECM Extracts for Wound Healing Effects in Cell Culture and Mouse Stromal Injuries

### Cell Culture Migration Assays

The HCO decellularized ECM extracted in PC is used on monolayer cell migration assays to determine their effect on primary donor corneal fibroblast and on the HCLE epithelial cell line migration. The data is quantified using imageJ.

### Corneal Wound Healing

One eye/wild type mouse (C57BL/6J) is injured with an Algerbrush® for a deep stromal wound in anesthetized mice (Vij, N., et al., Invest Ophthalmol Vis Sci, 2005. 46 (1): p. 88-95, Ghosh, A., et al., Invest Ophthalmol Vis Sci, 2009. 50 (9): p. 4185-91). HCO-ECM in fibrin gel or the latter alone as a control is applied to the injury site. HCO-ECM is also combined with PEGDA hydrogel (Garagorri, N., et al., Acta Biomater, 2008. 4 (5): p. 1139-47.). Wound healing and clearing of scar are tracked by in vivo slit lamp biomicroscopy between 0-6 weeks. Corneal swelling is determined using OCT. Mice are harvested at defined times and corneal sections are immunostained for a) ALDH3A1, KERA (keratocytes), THY1 (fibroblasts), vimentin, a-SMA (myofibroblast). Collagen III and fibronectin (regenerating ECM), SPARC (fibrosis) are immunostained for. Denatured/newly synthesized collagen is also stained for using CHP as described. Mouse corneas extracted at defined times are assessed for TGF-β intermediates, pro-inflammatory cytokines (mouse Th1/Th2 cytokine and chemokine multiplex) (Gowda, R. N., et al., PLOS One, 2015. 10 (9): p. e0137129, Shao, H., et al., PLOS One, 2013. 8 (1): p. e54765, Wu, F., et al., J Biol Chem, 2007. 282 (36): p. 26409-17).

### HCO-Derived Stromal Cells for Stromal Cell Therapy

It was hypothesized that HCO-derived stromal cells introduced into decellularized human corneas integrate and produce cornea-typical stromal ECM. The HCOs harbor stromal progenitor and fetal keratocyte-like cells which are likely to have regenerative potential. Stromal and progenitor cells were extracted from HCOs rapidly without monolayer expansion. Dye-labeled fibroblasts were injected into decellularized donor (DN) corneas, and their survival was tracked 10 days later. This approach is also used with HCO-stromal cells. HCO-stromal cells are injected into decellularized DN corneas, and long-term cell viability, differentiation into keratocyte, fibroblast, and myofibroblast are determined. TGF-β signaling and ECM gene expression in re-extracted HCO stromal cells from decellularized DN corneas is determined and compared to freshly isolated HCO-stromal cells and DN-stromal cells. Local ECM architecture of the DN corneas integrated with HCO-stromal cells is also determined.

HCO-stromal cells are introduced into decellularized DN corneas and the following are tested: A) cell viability, keratocyte, fibroblast, and myofibroblast marker expression, synthesis of cornea-typical ECM, collagens, glycoproteins and proteoglycans. scRNA-seq of HCO-cells maintained in decellularized corneas is compared with those freshly isolated from HCOs and native corneas to elucidate genes that underpin these cellular phenotypes and B) local ECM architecture of recipient donor corneas re-populated with HCO-stromal cell are elucidated by immunohistology and TEM using decellularized and native corneas as controls.

Cell-therapeutic potentials of HCO-cells are determined. HCO-cells' ability to integrate into a human donor cornea and produce cornea-typical ECM is tested. It was shown that rapid isolation of cells from HCOs enriched for stromal cells, such that there is no need for monolayer expansion. Second, human donor corneas were decellularized and dye-labeled primary stromal cells were introduced and tracked. This set up is used to functionally test HCO stromal cell-enriched pool.

Donor corneas were decellularized and injected with fibroblasts as described for porcine corneas (Chandru, A., et al., Biomolecules, 2021. 11 (4), Gonzalez-Andrades, M., et al., Invest Ophthalmol Vis Sci, 2011. 52 (1): p. 215-22). The DN corneas were kept in culture. Survival of injected cells was assessed after 10 days in culture. It was found that multiple injected cells remained in the recipient decellularized corneas. This approach is used to test the HCO stromal cells.

Gene expression supports the immature fetal quality of HCO stromal cells. The largest stromal cluster (CL2) in HCOs expresses cornea-like fibrillar collagens (COL1A1, COL1A2, COL5A1 ((2), but it also expresses COL3A1, fibronectin, COL6A1, LUM, and very low KERA, typical of an immature or regenerating corneal ECM. The smaller CL7 and CL10 clusters express ABCG2 and PAX6, consistent with keratocyte progenitor and precursor qualities (Du, Y., et al., Stem Cells, 2005. 23 (9): p. 1266-75, Funderburgh, M. L., et al., FASEB J, 2005. 19 (10): p. 1371-3). CL5 also expresses the proliferation markers. Thus, the HCO-stromal Clusters (CLs) have a good mix of progenitor and differentiating keratocytes which are predicted to have long lasting potential and producing maturing keratocytes in a native cornea. Two approaches were developed for enrichment of HCO stromal cells.

HCO stromal cells were enriched by differential adhesion and removal of epithelial/endothelial cells. Epithelial cells have been enriched for from 2D SEAM tissues by selective adhesion on the terminal fragment of laminin trimers (Hayashi, R., et al., Nature, 2016. 531 (7594): p. 376-380.). This approach was used for selective adhesion of the epithelial and endothelial pools to harvest the non-adherent stromal cell-enriched fraction. The adherent cells had a typical epithelial cobblestone appearance, while the non-adherent cells acquired a typical dendritic keratocyte-like phenotype in tissue culture plates. Taqman qRTPCR from these cultures showed that the “epithelial-like cells express the epithelial marker TP63, while the keratocyte-like cells express stromal cell markers KERA and LUM.

Stromal cells were enriched by magnetic-bead-based separation. In a second rapid HCO-stromal cell extraction approach, the finding that HCO stromal and progenitor cell CLs express VEGFR1 was utilized. By flow cytometry it was further confirmed that ~27% of the HCO cells are indeed VEGFR1+. For rapid enrichment of VEGFR1+ cells, HCO cells were incubated with PE-conjugated anti-VEGFR1, and EasySep Release Human PE positive selection was used to enrich this population. These cells showed keratocyte-like morphology in tissue culture. The magnetic bead extracted HCO stromal cells are directly introduced into decellularized human corneas without monolayer expansion.

Experimental designs for HCO-derived stromal cells for stromal cell therapy are now described.

Introduction of HCO Stromal Cells into Decellularized DN Corneas

Aged donor corneas are incubated 1.5 M NaCl for 48 hours, followed by PBS washes with antibiotics and DNase I digestion at 37 °C for 24 hours to remove nuclear material. HCO stromal cells are labeled with Vybrant DiO and 20,000 cells are injected into the decellularized human corneas. As controls low passage primary corneal keratocytes are injected into decellularized corneas. The cell-loaded corneas are placed in DMEMF12 with antibiotics and 10% FBS in 37 °C and 5% CO₂ incubator. After 5 days the media is switched to DMEMF12, antibiotics and in 1% FBS for up to 6 weeks.

### Phenotype of HCO Stromal Cells in Decellularized Human Cornea

At defined times (1, 2, 4, and 6 weeks) the corneas are fixed, OCT-embedded and immunostained for keratocyte, fibroblast and myofibroblast markers, and TUNEL stained as described to detect apoptotic cells. The HCO-cells are re-extracted from the DN corneas (DN-resident HCO cells), as controls DN central corneal cells (DN-cells) and the HCO-stromal cells are included for the following assessments: clonal expansion in culture, bulk RNA-seq to elucidate gene expression and phenotype. Local ECM architecture is elucidated.

### Ascertaining the Suitability of HCO Stromal Cells as a Standardized and Replenishable Source of Cells for Stromal Therapy

It was hypothesized that HCO-derived stromal cells promote scarless wound healing in mouse corneas. Debridement corneal wounding causes scarring in mice and applied DN cells resolve scars by releasing cytokines and proteins (Du, Y., et al., Stem Cells, 2009. 27 (7): p. 1635-42., Basu, S., et al Sci Transl Med, 2014. 6 (266): p. 266ra172). This technique was successfully tested, and it was shown that corneal fibroblasts resolve scarring by 2 weeks. It is expected that the HCO progenitor/fetal-keratocyte-like cells are not only able to resolve scars, but also survive and secrete corneal ECM proteins that are of relevance to corneal disease with ECM dysfunction when injected into undebrided corneas.

HCO-cells are applied on debrided mouse corneas and scar resolution in vivo is assessed along with wound healing in harvested corneas. HCO-stromal cells are injected into undebrided mouse corneal stroma and their long-term survival (4-6 weeks) along with their secretion of ECM is examined using available human-specific antibodies. Differentiation of the introduced HCO-cells into keratocytes, fibroblasts, and any myofibroblasts is assessed by immunohistology and flow cytometry. Stromal architecture of the introduced HCO-cells is assessed by confocal and electron microscopy.

It was hypothesized that HCO-derived stromal cells introduced into mouse corneas in vivo will promote wound healing. HCO-cells are applied on debrided mouse corneas and scar resolution in vivo as well as wound healing in harvested corneas is assessed. HCO-stromal cells are injected into undebrided mouse corneal stroma and their long-term survival (4-6 weeks) as well as their secretion of ECM is examined using available human-specific antibodies. Differentiation of the introduced HCO-cells into keratocytes, fibroblasts, and any myofibroblasts is assessed by immunohistology and flow cytometry. Stromal architecture is assessed by confocal and electron microscopy.

The HCO stromal cells have a mix of progenitor, proliferating and immature keratocyte-like cells, and therefore it was predicted these cells will survive and function as corneal stromal cells in the mouse cornea in vivo. For example, Basu et. al., showed that human limbal stromal cells applied on a debrided mouse cornea in fibrin gel, prevent scar formation and this was through modulation of neutrophil ingress into the cornea (Hertsenberg, A. J., et al., PLOS One, 2017. 12 (3): p. e0171712.). Other cell types, such as bone marrow, adipose tissue, umbilical cord, mesenchymal, and dental pulp have been similarly tested in mouse corneal scar resolution (Mittal, S. K., et al., Stem Cell Reports, 2016. 7 (4): p. 583-590, Zeppieri, M., et al., J Clin Med, 2017. 6 (12), Coulson-Thomas, V. J., et al., Stem Cells, 2013. 31 (10): p. 2116-26, Eslani, M., et al., Stem Cells, 2018. 36 (5): p. 775-784, Syed-Picard, F. N., et al., Stem Cells Transl Med, 2015. 4 (3): p. 276-85). This may become a valuable alternative to corneal transplants for scarring resulting from infections or injuries (Stepp, M. A., et al., Exp Eye Res, 2014. 121: p. 178-93, Koujah, L., R. K. Suryawanshi, and D. Shukla, Cell Mol Life Sci, 2018). Du et. al., showed that donor corneal limbal stem cells injected directly into the thin corneas of lumican-null mice produce ECM and restore thickness (Du, Y., et al., Stem Cells, 2009. 27 (7): p. 1635-42).

Lumican-nulls may be used for mouse-corneal wound healing in this example. Direct introduction of cells to the stroma is highly relevant to development of treatments of keratoconus (111), other ectasia (48) and stromal dystrophies (112-114) ( Soiberman, U., et al., Open Opthalmol J, 2017. 11: p. 252-261, Lee, J. Y., et al., Curr Opin Ophthalmol, 2023. 34 (3): p. 267-272, Aldave, A. J., et al., Clin Genet, 2013. 84 (2): p. 109-19, Klintworth, G. K., Front Biosci, 2003. 8: p. d687-713, Amberger, J. S., et al., Nucleic Acids Res, 2019. 47 (D1): p. D1038-D1043.).

### Mouse Debridement Corneal Scarring and DN Fibroblast Injection

One eye per mouse was injured with a debridement circular wound and Vybrant DiO-labeled DN fibroblasts were introduced and sealed with a fibrin sealant. In the control animal the debridement was treated with the fibrin sealant alone. The corneas were examined by slit lamp on day 0 and day 14 and then harvested for immunostaining of human COL5A1 (collagen type V subunit) to identify functional human keratocytes. The results showed that the injured cornea treated with cells resolved the scar. Immunohistology on day 14 showed a few cells COL5A1 expressing cells. Of note, the fully differentiated DN fibroblasts used here have limited progenitor potential and likely to disappear over time. It is expected that the HCO stromal cells, with progenitor and proliferative capacity, function over a longer period and this is evaluated.

This example identifies fibrosis-regulatory TGF-β signals in fetal cornea-like HCOs, a key to scarless wound healing. This example also develops HCO ECM as a tissue-regenerative biomaterial and its stromal cells as a standardized replenishable source for cell-therapy.

### The Human Corneal Organoid is a Powerful Model System

The cornea is the outermost layer of the eye, that provides barrier protection, transparency and 70% of the eye's refractive power (Maurice, D. M., J Physiol, 1957. 136 (2): p. 263-86, Hassell, J. R. and D. E. Birk, Exp Eye Res, 2010. 91 (3): p. 326-35, Meek, K. M. and C. Knupp, Prog Retin Eye Res, 2015. 49: p. 1-16). These functional demands are met by its three major cell types organized into a stratified epithelium, a thick highly organized stromal extracellular matrix (ECM) produced by keratocytes, and an innermost endothelium. Injuries, infections or congenital defects in these layers present the fourth largest cause of blindness worldwide, underscoring the urgent need for effective therapies. Corneal transplantation the primary treatment, critically, impact 12.7 million people who need corneal grafts (Ghebreyesus, T., World report on vision. 2019, World Health Organisation, Klintworth, G. K., Orphanet J Rare Dis, 2009. 4: p. 7, Porth, J. M., et al., Cornea, 2019. 38 (12): p. 1602-1609, Kate, A. and S. Basu, F1000Res, 2023. 12: p. 1309, Lobo, A. M., et al., Ocul Surf, 2019. 17 (1): p. 40-49, Gain, P., et al., JAMA Ophthalmol, 2016. 134 (2): p. 167-73). With the beginning of self-organizing organoids from pluripotent stem cells, as in the cerebral cortex and the gut, a revolutionary model system arose to study cells in a 3D environment that partially mimicked a human organ. Early organoids were developed from human embryonic(ES) and rare tissue stem cells (Eiraku, M., et al., Cell Stem Cell, 2008. 3 (5): p. 519-32, Sato, T., et al., Nature, 2009. 459 (7244): p. 262-5, Nakano, T., et al., Cell Stem Cell, 2012. 10 (6): p. 771-85). But reprogramming of somatic cells to induce pluripotent stem cells (iPSC) has brough the organoid field close to patient-specific clinical and therapeutic research (Takahashi, K., et al., Cell, 2007. 131 (5): p. 861-72.). Following iPSC-derived optic and retinal cup and lacrimal gland organoids, 3D human cornea organoids (HCOs) were developed which mimic rudimentary corneal layers and collagen fibril stacks (Foster, J. W., et al., Sci Rep, 2017. 7: p. 41286, Wahlin, K. J., et al., Scientific Reports, 2017. 7 (1): p. 766, Hayashi, R., et al., Nature, 2022. 605 (7908): p. 126-131, Bannier-Helaouet, M., et al., Cell Stem Cell, 2021. 28 (7): p. 1221-1232 e7). Single cell RNA sequence analysis further revealed epithelial, endothelial, and stromal cell clusters in 4-month-old HCOs. HCOs were developed using multiple lines to address their development as a source of regenerative ECM and stromal cells for future therapy. Characterization of HCOs elevates their use in functional studies of disease genes before extensive use of animal models.

### Key Advantages in the HCOs Over Existing 3D Cell Culture Models of Single Differentiated Cell Types

3D spheroid, pellet, and scaffolded cultures use one or two primary cell types, extracted from cadaverous donor tissue, and this vulnerable to donor-specific heterogeneity and rapid aging (Garagorri, N., et al., Acta Biomater, 2008. 4 (5): p. 1139-47, Scott, S. G., et al., Exp Eye Res, 2011. 93 (6): p. 898-905, Uchida, S., et al., Invest Ophthalmol Vis Sci, 2005. 46 (5): p. 1620-5, Funderburgh, M. L., et al., Mol Vis, 2008. 14: p. 308-17, Du, Y., et al., Invest Ophthalmol Vis Sci, 2007. 48 (11): p. 5038-45, Karamichos, D., et al., J Tissue Eng Regen Med, 2011. 5 (8): p. e228-38, Priyadarsini, S., et al., PLOS One, 2016. 11 (12): p. e0168845). Instead, the HCOs, derived from iPSC lines, are less variable and more tolerant to cell-passaging, representing a virtually limitless source. The co-differentiation of all corneal cell types in HCOs retrace early corneal development making HCOs a more physiologically relevant model (Cvekl, A. and E. R. Tamm, Bioessays, 2004. 26 (4): p. 374-86, Graw, J., Eye development. Curr Top Dev Biol, 2010. 90: p. 343-86).

Compared to Other iPSC-Derived Corneal Tissues, HCOs have High Stromal Cell Count and Robust Proteoglycan/Collagen Presence Resembling the Corneal Stroma

Ocular organoid development relies on the principle that cell aggregates exposed to early eye-field signals such as, Wnt signal inhibition, B27, smoothened agonist, recapitulate eye development to produce ectoderm and neuroectoderm (Walker, H., et al., Exp Eye Res, 2020. 201: p. 108231, Lwigale, P. Y., Prog Mol Biol Transl Sci, 2015. 134: p. 43-59). This approach naturally favors epithelial lineage that was leveraged to produce self-formed ectodermal autonomous multi-zone (SEAM) as 2-dimensional tissues for epithelial cell therapies (Hayashi, R., et al., Nature, 2016. 531 (7594): p. 376-380). The modified cues that were used in HCO generation yielded organoids with epithelial and stromal cells and more cornea-like stromal collagens and proteoglycans (Foster, J. W., et al., Sci Rep, 2017. 7: p. 41286, Maiti, G., et al., PNAS Nexus, 2022. 1 (5): p. pgac246, Foster, J. W., et al., Methods Mol Biol, 2020. 2145: p.51-58, Ashworth, S., et al., Ocul Surf, 2025. 35: p. 68-80). Two other cornea organoids are also epithelium-rich (Susaimanickam, P. J., et al., Development, 2017. 144 (13): p. 2338-2351, Swarup, A., et al., Stem Cell Reports, 2023. 18 (12): p. 2482-2497). Thus far cell-based treatments for epithelial and endothelial conditions, have advanced (Rama, P., et al., N Engl J Med, 2010. 363 (2): p. 147-55, Yazdanpanah, G., S., et al., Curr Ophthalmol Rep, 2019. 7 (1): p. 1-10, Pellegrini, G., et al., Regen Med, 2016. 11 (4): p. 407-20, Shortt, A. J., et al., Br Med Bull, 2011. 100: p. 209-25, Bonnet, C., S., et al., Stem Cells Transl Med, 2023. 12 (11): p.714-719, Kinoshita, S., et al., N Engl J Med, 2018. 378 (11): p. 995-1003, Zhu, Y. T., et al., Ocul Surf, 2023. 29: p. 301-310). The stroma-rich HCOs developed in this example addresses the existing gap in stromal ECM and cell-based therapies of the stroma. The keratocyte-like cells in the HCOs develop in close proximity with epithelial and endothelial cells as in a native 3D corneal environment and secrete keratan sulfate (KS) and chondroitin sulfate (CS) proteoglycans and fibrillar collagens, the hallmarks of a cornea-like stroma (Ashworth, S., et al., Ocul Surf, 2025. 35: p. 68-80). In this example, the HCO stroma however was shown to mimic an immature developing stroma, a quality that may be harnessed to use the HCO stroma as a stromal matrix, like the widely used murine Matrigel® has become. Importantly, being of human source, the decellularized HCO ECM of this example may have clinical wound healing applications.

### HCOs Mimic Immature Human Fetal Corneas and are Potentially a Source of Stromal Cells for Therapeutic Purposes

A Metaneighbor comparison of HCO scRNA-seq data with that of a published report on human fetal cornea stages was used to reveal closer alignment of the HCOs with 17-18 and 20-21 post conception week fetal stages (Ashworth, S., et al., Ocul Surf, 2025. 35: p. 68-80, Collin, J., et al., Ocul Surf, 2021. 21: p. 279-298). The HCO stromal cells share high similarity scores with fetal keratocytes and fibroblasts, and like multipotent human fetal keratocytes, the HCO stromal cells may be ideal for cell-based therapies (Chao, J. R., et al., Stem Cells Dev, 2013. 22 (15): p. 2186-95). Currently, stromal cell therapy attempts to include mesenchymal/stromal stem cells for treating corneas undergoing steroid-resistant graft versus host disease, but these have yet to reach major therapy modalities (Le Blanc, K., et al., Lancet, 2008. 371 (9624): p. 1579-86, Lee, J. Y., et al., Curr Opin Ophthalmol, 2023. 34 (3): p. 267-272). Autologous adipose derived stem cell therapy was tested in a few keratoconus patients, but patient-specific challenges may prevent its widespread use (El Zarif, M., et al., Front Med (Lausanne), 2021. 8: p. 650724). Directly deriving keratocyte-like cells from iPSC lines has yielded mixed results. Attempts to produce intermediate neural crest-like cells have used subsequent passage through cadaveric corneal limbal rims to generate keratocytes which is not ideal for eventual use in patients (Naylor, R. W., et al., PLOS One, 2016. 11 (10): p. e0165464). Patient-derived iPSCs have also been taken through a forced-aggregation step to yield “neural-crest-like” cells to keratocytes but not secreted ECM was examined (Nayak, D., et al., Stem Cell Res Ther, 2024. 15 (1): p. 464).

TGF-β Signals were Shown to Regulate the ECM Production Versus Fibrosis Balance and its Components were Detectable in the HCO

The TGF-β signals promote epithelial and stromal wound healing responses in the cornea, including epithelial-mesenchymal transition (EMT), mesenchymal to epithelial transition, cell migration and wound healing (Kawakita, T., et al., J Cell Physiol, 2013. 228 (1): p. 225-34, Wang, Y. C., et al., Ocul Surf, 2023. 29: p. 557-565, Wang, Y. C., et al., Cells, 2024. 13 (7)). These signals have profound effects of keratocyte, activated fibroblast, and myofibroblast differentiation that orchestrate corneal repair or fibrosis. The HCOs are a unique 3D system to address the epithelial and fibroblast response in a versatile, tractable system. Importantly, scRNA-seq of HCOs was shown in this example to indicate expression of all major TGF-β signaling components (Maiti, G., et al., PNAS Nexus, 2022. 1 (5): p. pgac246, Ashworth, S., et al., Ocul Surf, 2025. 35: p. 68-80).

The TGF-β ligands (helped by the co-receptor betaglycan) are presented to the type II TGFBR2 receptor which phosphorylates (p) the type I TGFBR1 at serine/threonine. The pTGFBR1 phosphorylates the transcription factors, SMAD2/3 or SMAD1/5/8 (R-SMADs) and the co-SMAD4, which translocate to the nucleus and regulate gene expression. Additional control, including many co-transcription factors, and context-dependent fine-tuning occurs at multiple levels. TGF-β cleaved in the Golgi remains associated with its non-signaling N-terminal latency-associated peptide (LAP) as an inactive complex. The LTBPs, ECM proteins (thrombospondin 1, fibrillin1), integrins (αvβ6) and MMPs, interact with or degrade LAP to release the active ligand. Although the three TGF-β isoforms interact with the same receptors, their differential expression and storage of latent forms may have different biological consequences. It was found that there is increased TGF-β2 and pSMAD2 presence in the epithelium and stroma of human keratoconus corneas (Engler, C., et al., Am J Ophthalmol, 2011. 151 (5): p. 752-759 e2). In the cornea TGF-β1 and TGF-β2 may be fibrotic and TGF-β3 possibly anti-fibrotic, but this is far from settled (Karamichos, D., et al., J Tissue Eng Regen Med, 2011. 5 (8): p. e228-38, Karamichos, D., et al., Exp Eye Res, 2014. 124: p. 31-6, Wilson, S. E., Exp Eye Res, 2021. 207: p. 108594). The importance of TGF signals in corneal homeostasis was demonstrated in mice carrying keratocyte-specific knockouts (KO) of tgfbr2, smad4, tgfbr1; all three develop stromal ECM loss and keratoconus-like ectasia, but the most severe phenotype in the Tgfbr1 KO, may be due to poor ECM synthesis and an unusual Smad4 dependent ECM degradation, as a second Smad4 KO alleviated ECM degradation (53, 54, 58) (Wang, Y. C., et al., Ocul Surf, 2023. 29: p. 557-565, Wang, Y. C., et al., Cells, 2024. 13 (7), Yuan, Y., et al., Ocul Surf, 2025.). It was found in this example that stromal keratocyte cultures from human keratoconus corneas rapidly phosphorylate SMAD 1/5/8 after TGF-β1 stimulation, while the more common pSMAD2/3 axis was constitutively active (Foster, J., et al., PLOS One, 2014. 9 (9): p. e106556.). It is possible that these two pSMAD4-dependent signals have different consequences on ECM synthesis and degradation. The HCO is a simpler model to investigate TGF-β regulated ECM deposition, degradation and fibrotic changes.

### Keratocyte, Fibroblast, and Myofibroblast Phenotypes, Key to Corneal Health And Fibrosis, are Regulated by TGF-β Signals

The quiescent and differentiated keratocytes maintain the corneal stromal ECM (Hassell, J. R. and D. E. Birk, Exp Eye Res, 2010. 91 (3): p.326-35, Poole, C. A., et al., J Cell Sci, 1993. 106 (Pt 2): p. 685-91, Muller, L. J., et al., Invest Ophthalmol Vis Sci, 1995. 36 (13): p. 2557-67, Watsky, M. A., Invest Ophthalmol Vis Sci, 1995. 36 (13): p. 2568-76, Yam, G. H. F., et al., Exp Eye Res, 2020. 196: p. 108062, Espana, E. M. and D. E. Birk, Exp Eye Res, 2020. 198: p. 108137). Injury and surgery, cause apoptosis of damaged keratocytes, and activation of neighboring cells to acquire immature keratocyte or activated fibroblast and myofibroblasts phenotypes. These cellular changes that cause loss of corneal crystallins (65-67), secretion of profibrogenic ECM and corneal haze (66, 68-74) (Stepp, M. A., et al., Exp Eye Res, 2014. 121: p. 178-93, Ljubimov, A. V. and M. Saghizadeh, Prog Retin Eye Res, 2015. 49: p. 17-45, Jester, J. V., et al., J Cell Sci, 1999. 112 (Pt 5): p. 613-22, Zieske, J. D., Curr Opin Ophthalmol, 2001. 12 (4): p. 237-41, Wilson, S. E., Trans Am Ophthalmol Soc, 2002. 100: p. 411-33, Vij, N., et al., Invest Ophthalmol Vis Sci, 2005. 46 (1): p. 88-95, Beecher, N., et al., Invest Ophthalmol Vis Sci, 2006. 47 (1): p. 146-50, Petroll, W. M., et al., Invest Ophthalmol Vis Sci, 2004. 45 (10): p. 3466-74, Torricelli, A. A., et al., Exp Eye Res, 2016. 142: p. 110-8, Foulsham, W., et al., Ocul Surf, 2019. 17 (3): p. 374-383).

The adult corneal stromal ECM at rest is comprised of uniformly organized fibrils of collagen types I and V (collagen type III in developing corneas), anionic proteoglycans, lumican and keratocan, modified with keratan sulfate (KS), and decorin and biglycan with chondroitin sulfate (CS) glycosaminoglycans (GAGs), essential for corneal structure hydration, transparency and refractive (Hassell, J. R. and D. E. Birk, Exp Eye Res, 2010. 91 (3): p. 326-35, Meek, K. M. and C. Knupp, Prog Retin Eye Res, 2015. 49: p. 1-16, Espana, E. M. and D. E. Birk, Exp Eye Res, 2020. 198: p. 108137, Liu, C. Y., et al., J Biol Chem, 2003. 278 (24): p. 21672-7, Caterson, B. and J. Melrose, Glycobiology, 2018. 28 (4): p. 182-206, Chakravarti, S., et al., J Cell Biol, 1998. 141 (5): p. 1277-86, Chakravarti, S., Glycoconj J, 2002. 19 (4-5): p. 287-93, Chakravarti, S., et al., Invest Ophthalmol Vis Sci, 2003. 44 (6): p. 2422-32). Studies show that TGF-β signals promote keratocyte to fibroblast to myofibroblast transition in culture (Beales, M. P., et al., Invest Ophthalmol Vis Sci, 1999. 40 (8): p. 1658-63, Jester, J. V., et al., Cornea, 1996. 15 (5): p. 505-16). Gene expression patterns underlying the keratocytes, fibroblasts and myofibroblast phenotypes have been examined (82-85) (Chakravarti, S., et al., Invest Ophthalmol Vis Sci, 2004. 45 (10): p. 3475-84, Wu, F., et al., Exp Eye Res, 2008. 87 (3): p. 214-25, Funderburgh, J. L., et al., J Biol Chem, 2001. 276 (47): p. 44173-8, Norman, B., et al., Invest Ophthalmol Vis Sci, 2004. 45 (2): p. 429-40). In this example, these gene expression patterns are used to track the cellular phenotypes and fibrotic response in TGF-β treated HCOs and cell fates of HCO cells maintained in decellularized human corneas and in vivo mice.

It is found that development of an organoid-derived matrix can advance wound healing in the clinic. Studies of the TGF-β network in HCOs elucidate antifibrogenic nodes that can be targeted for wound repair. Investigations of the stromal cell bring a novel replenishable source for cell therapy. Deep characterization of HCOs leads to use of HCOs for functional studies of disease genes negating the need to use donor corneas and model animals.

### a Novel Model of the Human Cornea

3D HCOs were developed from iPSCs, creating a novel in vitro model that mimics the features of the developing human cornea. This provides a scalable and reproduceable alternative to traditional models that rely on cadaveric donor tissue. The HCO model is capable of producing stromal cells. The HCO model can also functionally test ocular disease genes before extensive animal model usage.

### a Platform for Studying TGF-β Signaling

The HCO model is an innovative platform to probe fibrosis-regulatory aspects of TGF beta signals. This network is central to controlling epithelial to mesenchymal transition, mesenchymal to epithelial transition, and differentiation to keratocytes, fibroblast and myofibroblasts during wound healing. These cellular phenotypes and their consequences are studied in this simplified model without the confounding effects of immune cells which are minimal in the HCO. However, the HCO model allows for the controlled addition of immune cells and other cell types in co-culture experiments, enabling the study of interactions between these cells and the corneal cells in a more complex and physiologically relevant environment.

a Source of Cells with Enhanced Regenerative Potential

The fetal cornea-like nature of the HCOs, harboring a mix of stem, progenitor and immature stromal cells, presents an unprecedented opportunity for developing cell-based therapies for corneal diseases. This is a standardized, scalable source of cells with enhanced regenerative potential, compared to those isolated from donor tissues.

### a Novel Biomaterial for Corneal Repair

The HCO ECM, which mimics features of an immature regenerative corneal matrix, offers a novel biomaterial for wound healing and stromal repair. The decellularized HCO matrix is used for wound healing and as a filler for stromal injuries. The HCO ECM is a cutting-edge new tissue for regenerative treatments.

### Example 3: Human Corneal Organoids for ECM and Cell Therapy and Pre-Animal Model System

The human cornea organoids are fetal cornea-like in having an immature ECM and a mix of progenitor and fetal keratocyte-like cells. It was hypothesized that the regenerative ECM and the fetal cornea-like stromal cells of HCOs can be developed into tools for scarless wound healing. It was hypothesized that the TGF-β network known to control cell fates, healthy and fibrotic ECM states, regulates this immature regenerative state of the organoid. It was also hypothesized that the regenerative ECM and the fetal cornea-like stromal cells of the human cornea organoids can be used as tools for scarless wound healing. It was also hypothesized that the human cornea organoids are a surrogate model of the cornea for functional studies of disease genes.

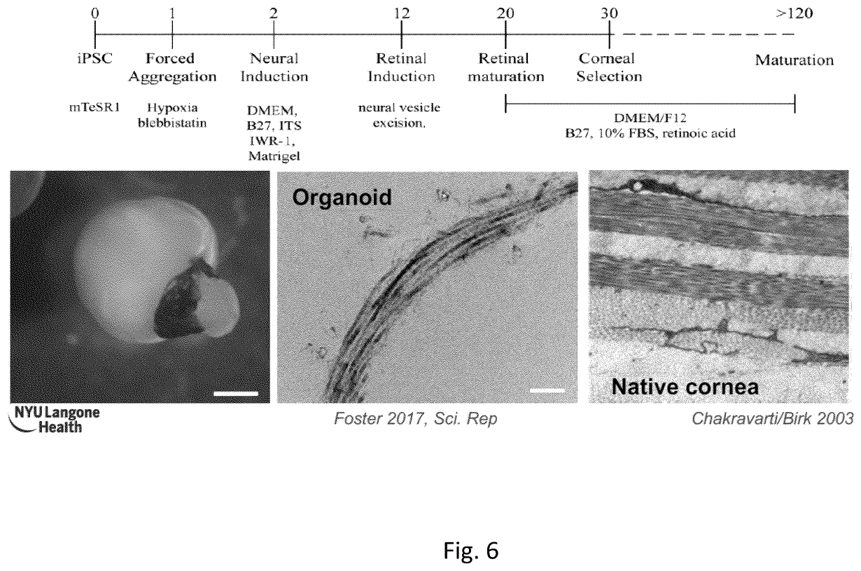

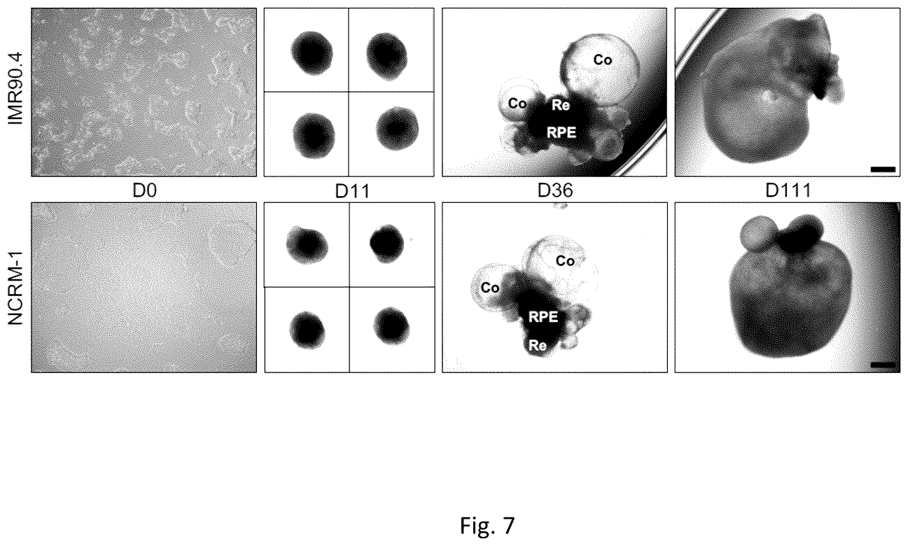

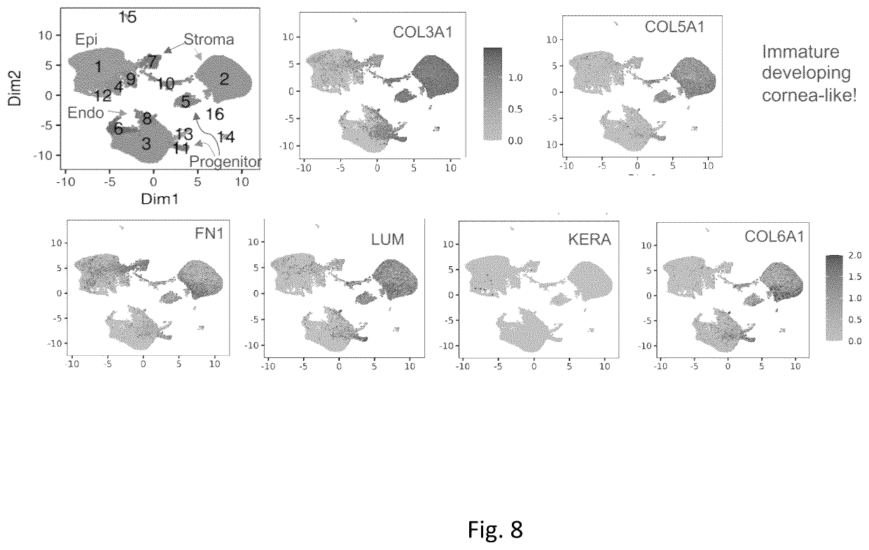

A protocol and timeline for developing human cornea organoids from iPSCs was developed (FIG. 6). Cornea organoids were developed from multiple iPSC lines (FIG. 7) (Ashworth, Ocul, Surf., November 2024). The stroma of the human cornea organoids is of an immature/remodeling nature. scRNA-seq data that shows that stromal genes are expressed in the human cornea organoids and that the human cornea organoids are similar to immature developing cornea (FIG. 8) (Maiti, PNASnexus, 2022).

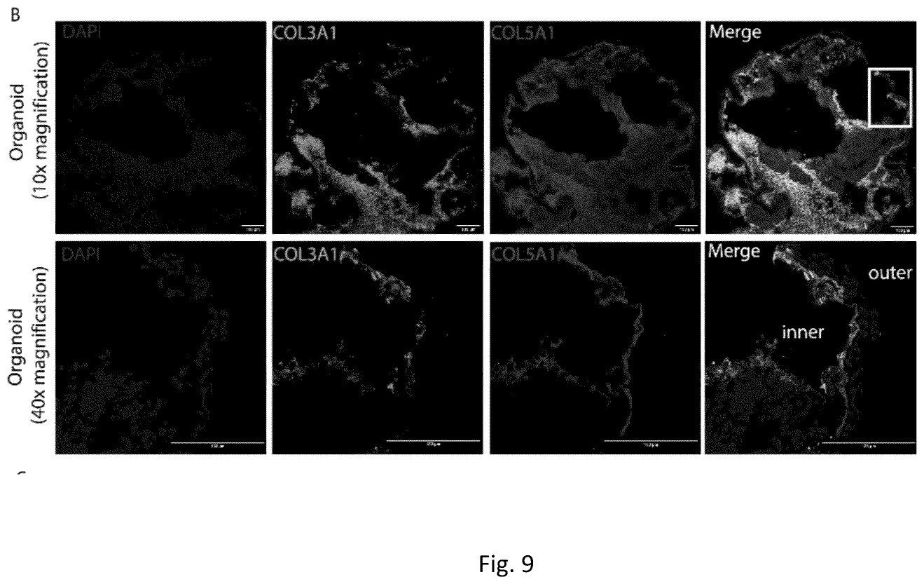

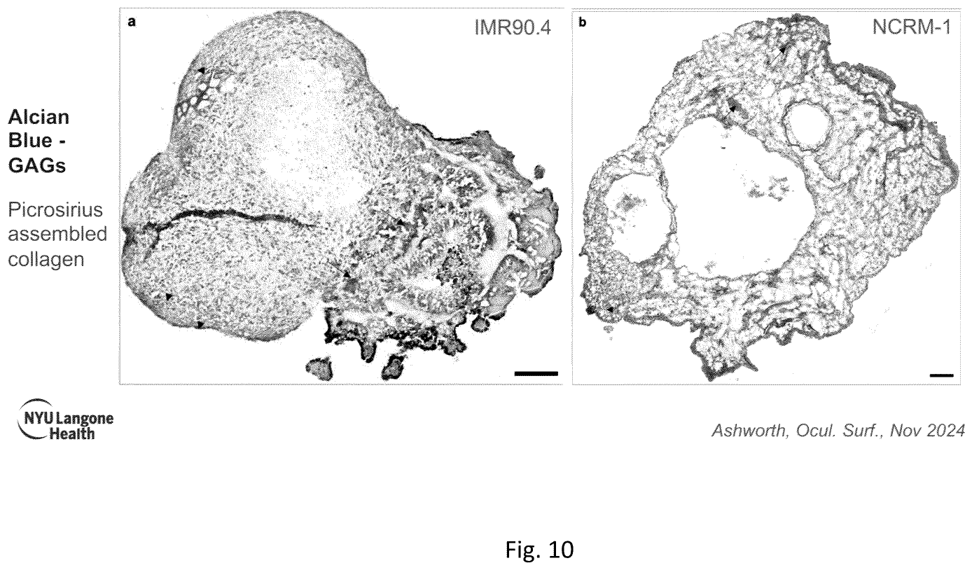

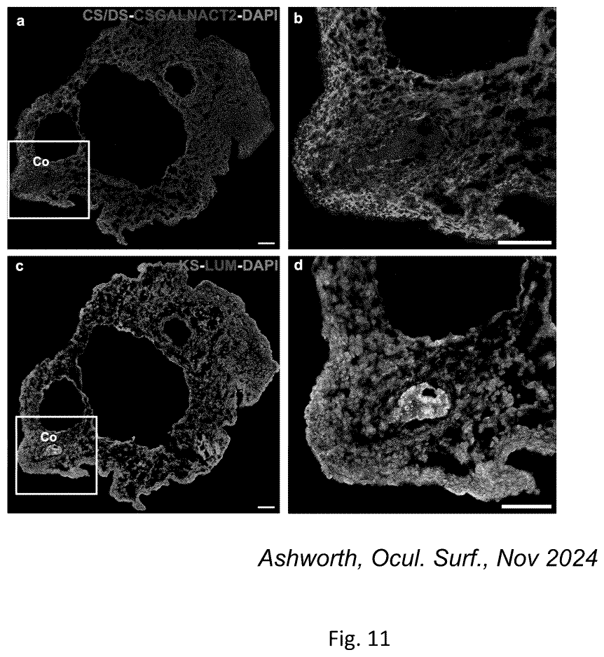

Human cornea organoids contain a collagen type II and V-rich stromal ECM (FIG. 9) (Maiti, PNASnexus, 2022). Human cornea organoids were also stained for fibrillar collagen and GAG (FIG. 10), and found to contain stromal proteoglycan and GAGs (FIG. 11) (Ashworth, Ocul, Surf., November 2024). Human cornea organoids had slightly more CS than KS which is indicative of immaturity (FIG. 11).

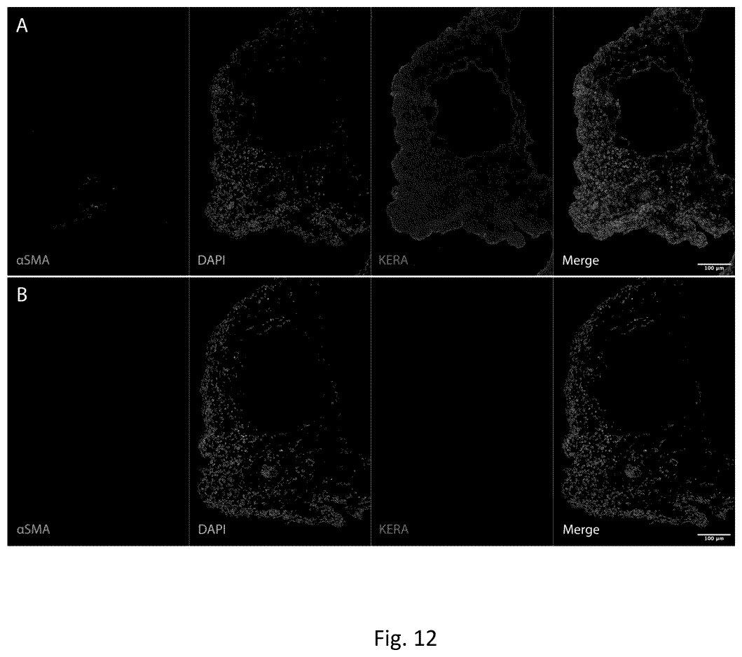

Keratocan was deposited in the human corneal organoid stroma (FIG. 12).

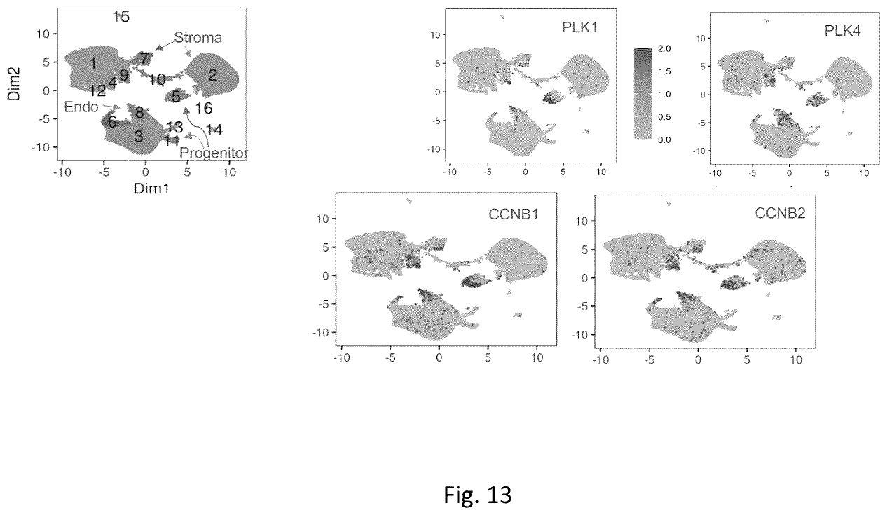

Human corneal organoids had progenitor/proliferating immature keratocyte-like cells shown by scRNA-seq data (FIG. 13). Ser/Thr kinases promote cell proliferation. Cyclin B1 and Cyclin B2 regulate TGF-beta mediated cell proliferation.

Organoid stromal cells were found to resemble developing corneal keratocytes and fibroblasts through a MetaNeighbor analysis of HCO scRNA-seq data and a human fetal corneal study (FIG. 1) (Collin 2021, Ocul. Surf, Ashworth, 2024, Ocul. Surf., Maiti, PNASnexus, 2022).

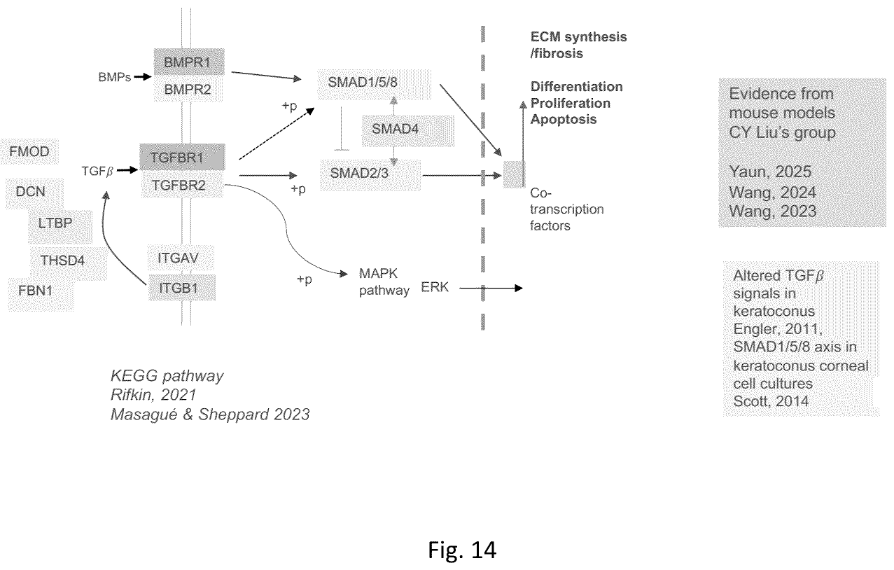

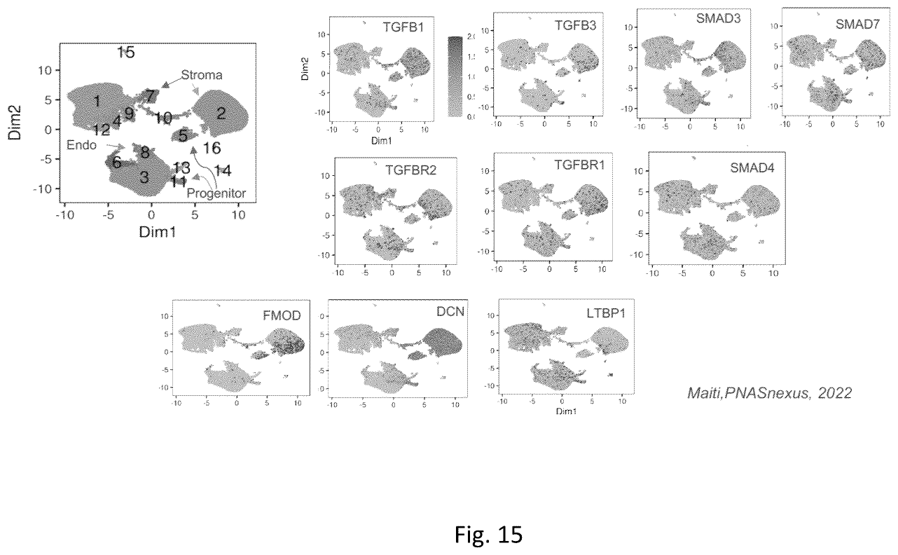

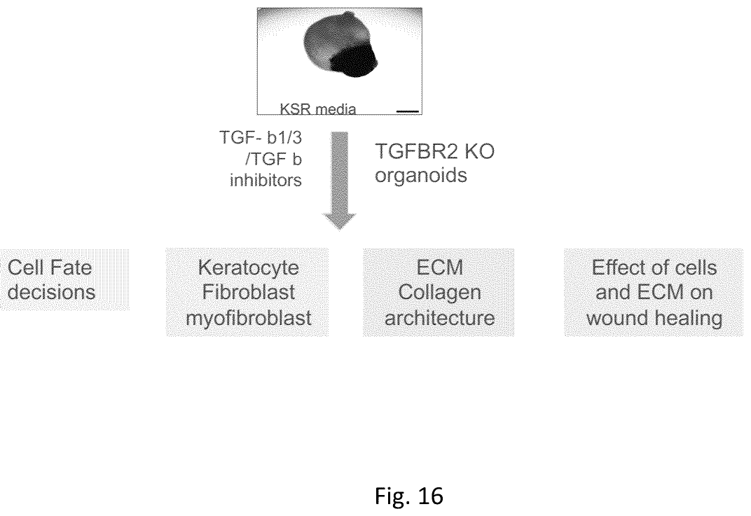

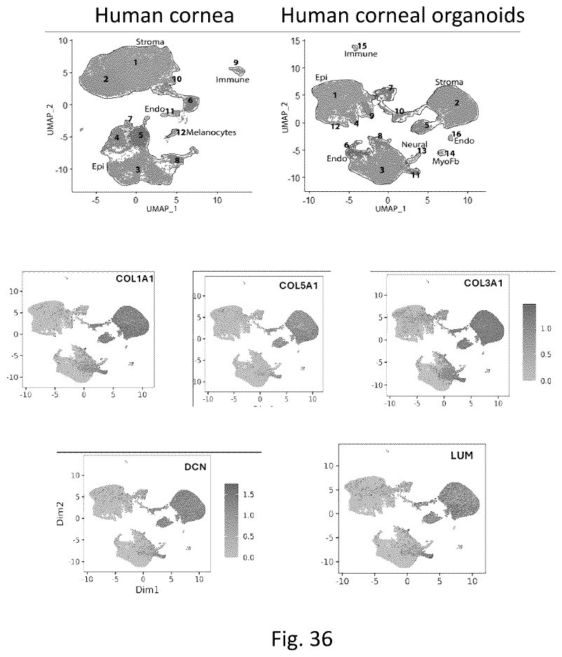

The corneal organoid is a useful model for understanding pro-regenerative TGF-β signals. TGF-β signaling regulates healthy and fibrotic ECM. TGF-β signaling is key to healthy ECM build up and fibrosis (FIG. 14) (KEGG Pathway, Rifkin, 2021, Masague & Sheppard 2023, Yuan 2025, Wang 2024, wang 2023, Engler 2011, Scott 2014). Key TGF-β signal mediators were expressed in the human cornea organoids shown by scRNA-seq data (FIG. 15) (Maiti, PNASnexus, 2022). ECM-regenerative TGF-β signals are tested in HCOs (FIG. 16). Major corneal ECM genes were expressed in the human cornea organoids shown by scRNA-seq data (FIG. 36). The effects of TGF-β signaling on cell fate decisions, keratocytes, fibroblasts, myofibroblasts, ECM architecture, collagen architecture, and on wound healing are tested.

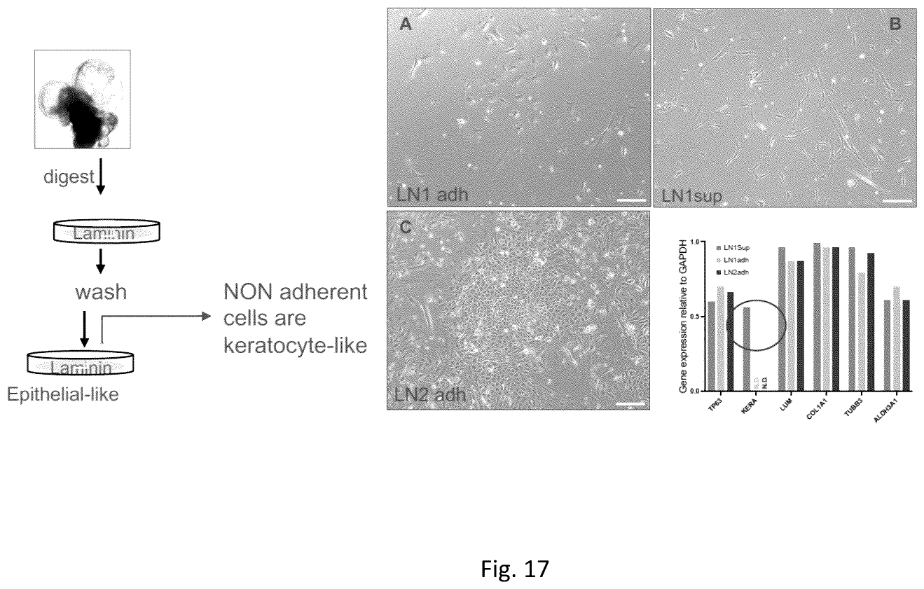

Human corneal organoid stromal cells and human corneal organoid derived ECM can be used for therapy due to the immature fetal cornea-like quality of HCOs. For example, HCO components can have regenerative wound healing potential. A protocol for human cornea organoid stromal cell rapid extraction was developed (FIG. 17). It was found that non-adherent cells are keratocyte-like (FIG. 17). Human cornea organoids were digested and cells were placed on laminin dishes and washed. Epithelial-like cells remained on the plate and the non-adherent cells were keratocyte-like (FIG. 17).

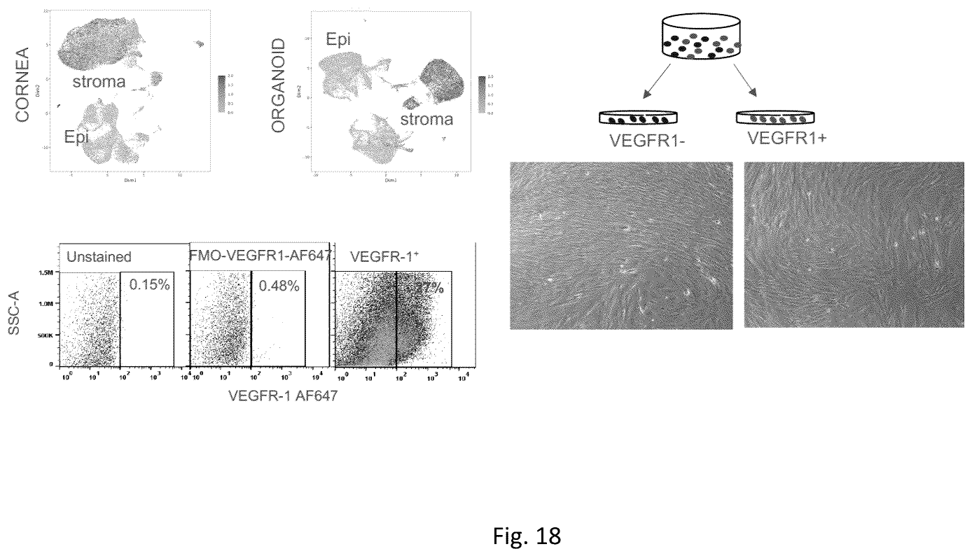

Another protocol for rapid extraction of stromal cells was developed using magnetic beads, and human cornea organoid cells were sorted based on VEGFR1 (FIG. 18).

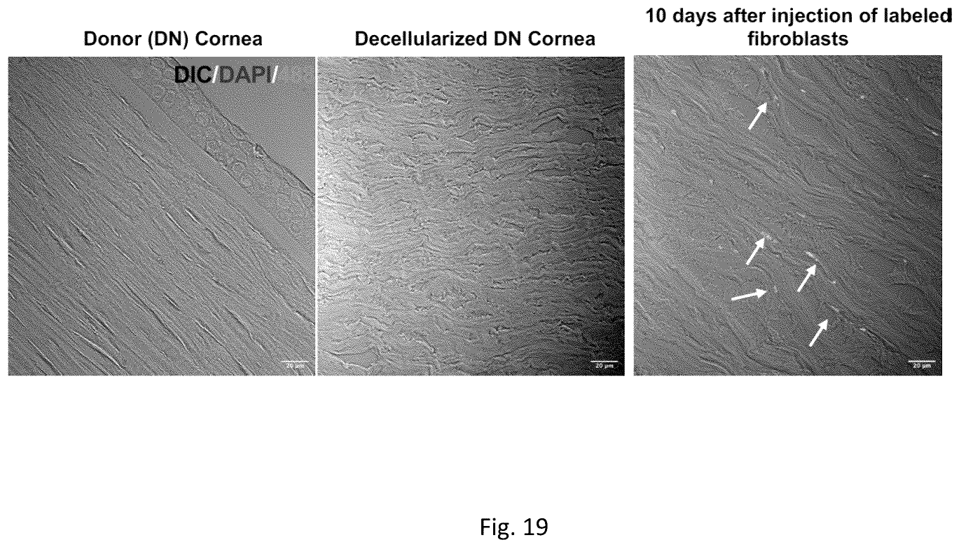

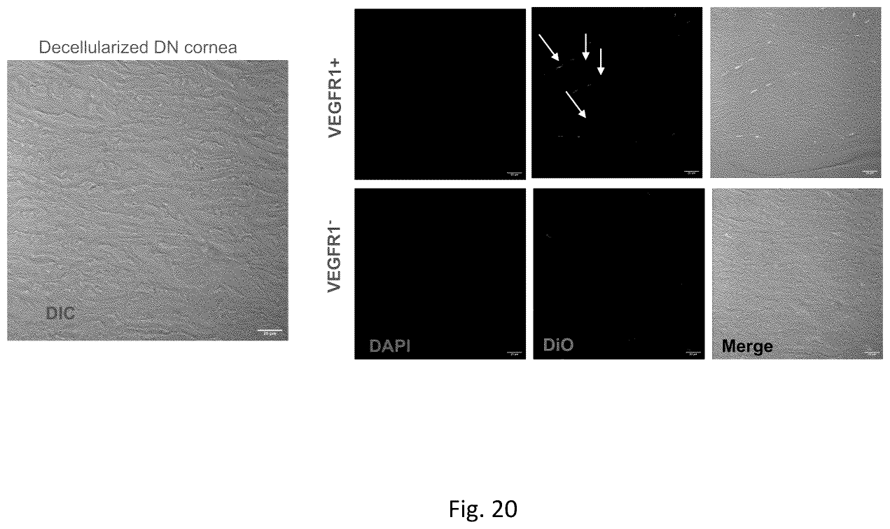

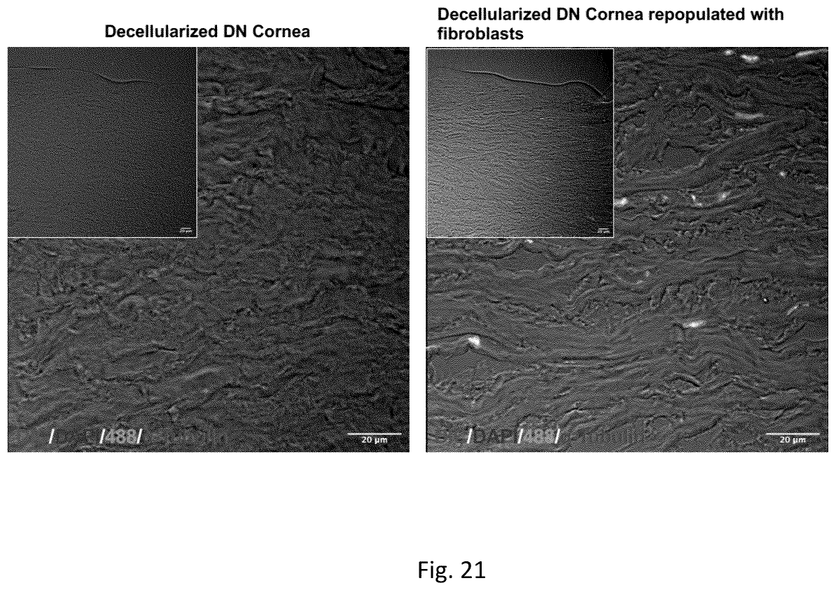

Human cornea organoids were functionally tested in human and mouse corneas. Labeled fibroblasts were introduced to decellularized donor corneas and tracked (FIG. 19). Labeled fibroblasts were found in decellularized donor corneas 10 days after injection (FIG. 19). HCO derived VEGFR1+ and VEGFR1-cells were integrated into donor corneas (FIG. 20). Fibroblasts that were introduced to the decellularized DN cornea became well-integrated in the decellularized DN cornea (FIG. 21).

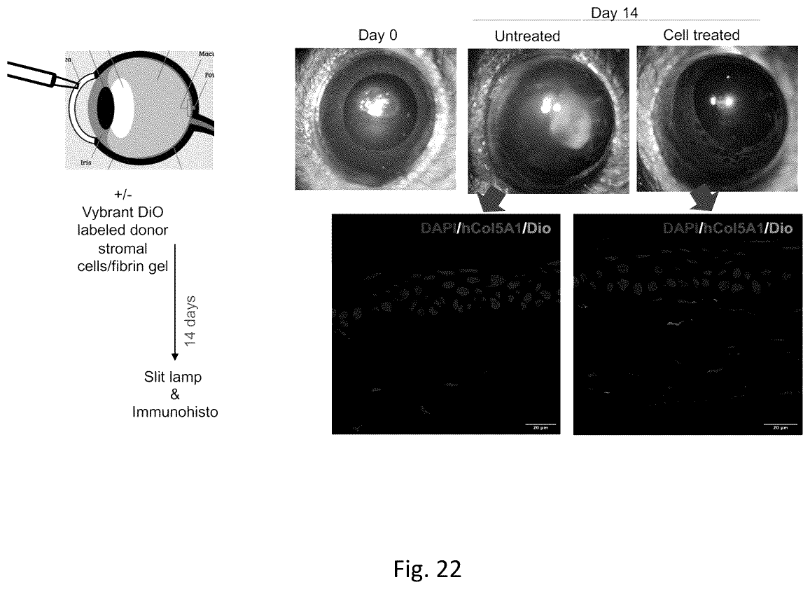

Donor (DN) fibroblasts were delivered to injured mouse corneas (FIG. 22). Vybrant DiO labeled donor stromal cells were delivered to injured mouse corneas. After 14 days injured mouse corneas that were cell treated were compared to untreated injured mouse corneas. Slit lamp and immunohistology was used to visualize the integration of stromal cells into injured mouse corneas.

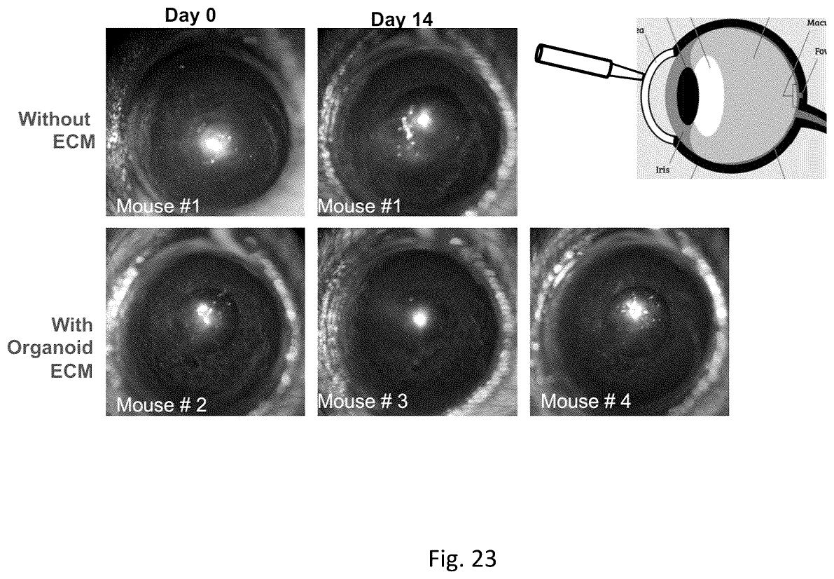

HCO ECM was combined with a fibrin gel and applied to mouse corneas (FIG. 23)

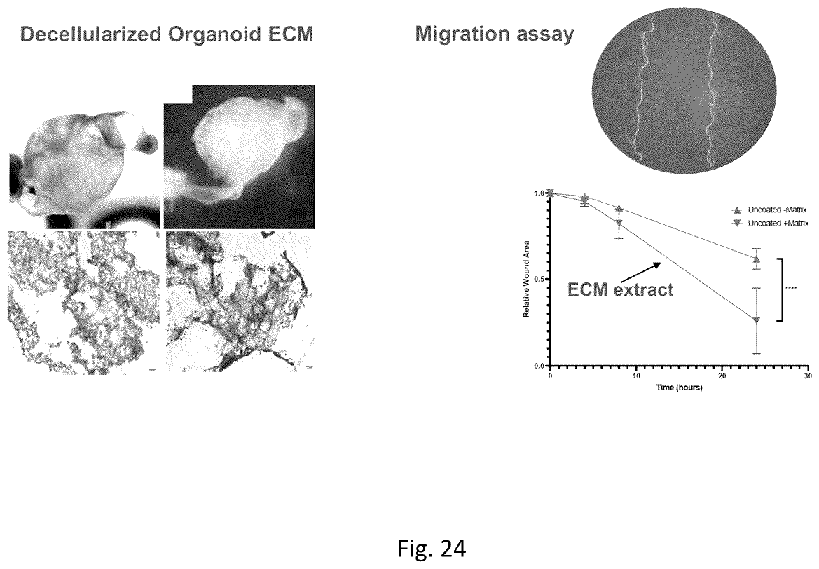

Corneal organoid ECM extract was found to promote cell migration (FIG. 24). Decellularized HCO ECM was used in a cell migration assay. Relative wound area decreased when ECM extract was used in the assay.

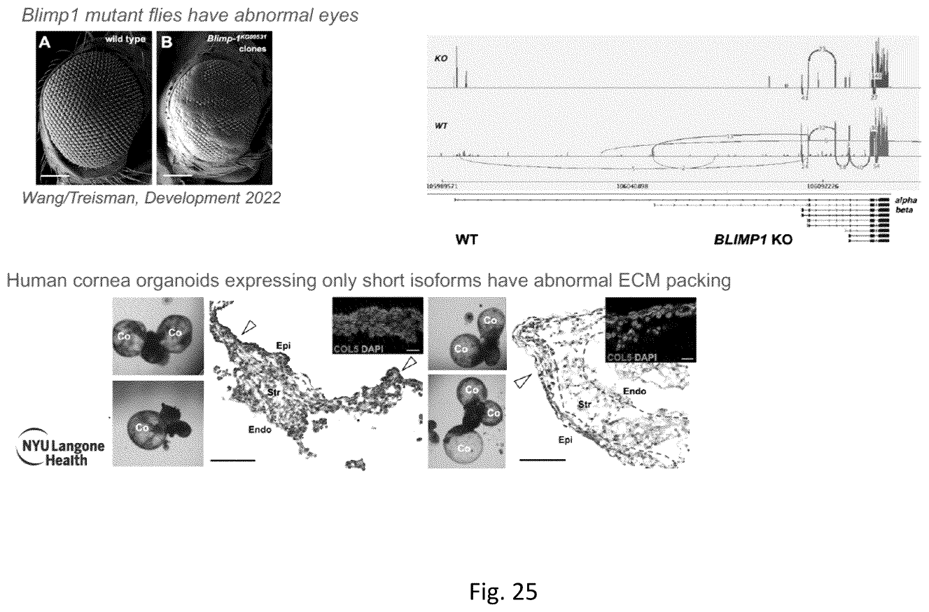

Human cornea organoids are used as surrogate models for disease genes. Blimp1/Prdm 1 transcription factors were found to be expressed in human cornea organoids (FIG. 25). Human cornea organoids expressing only short isoforms of Blimp1/Prdm1 were found to have abnormal ECM packing (FIG. 25).

The human cornea organoids were found to resemble fetal corneas. TGF-β network genes were found to be expressed in the human cornea organoids. The human cornea organoid ECM and the human cornea organoid stromal cells were found to have properties that may support wound healing. The human cornea organoids were found to be a model for functional studies of corneal disease genes. HCOs from keratoconus genetic variants may be tested. HCO ECM and HCO stromal cells may be a source of standardized non-murine ECM tissue and cell-based tools.

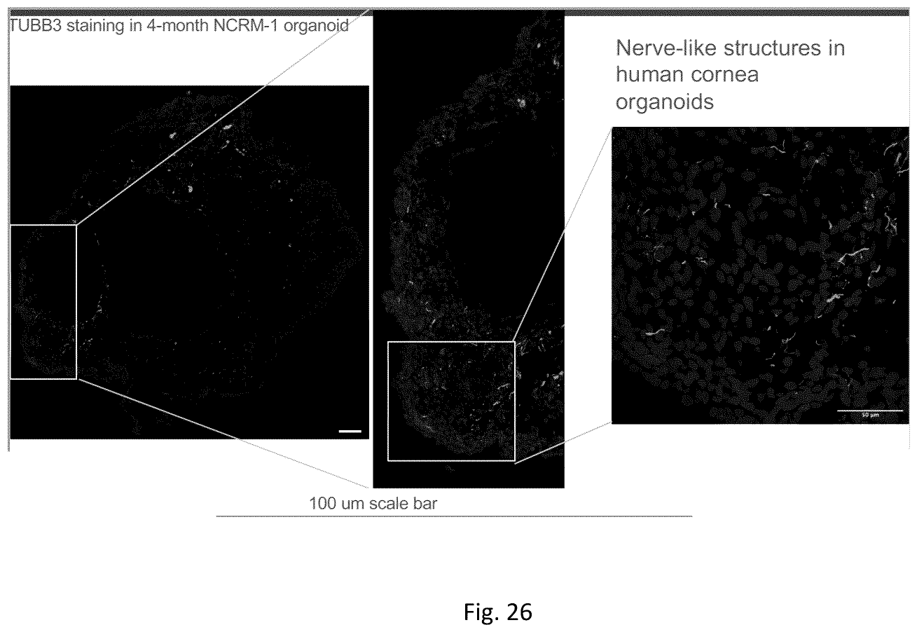

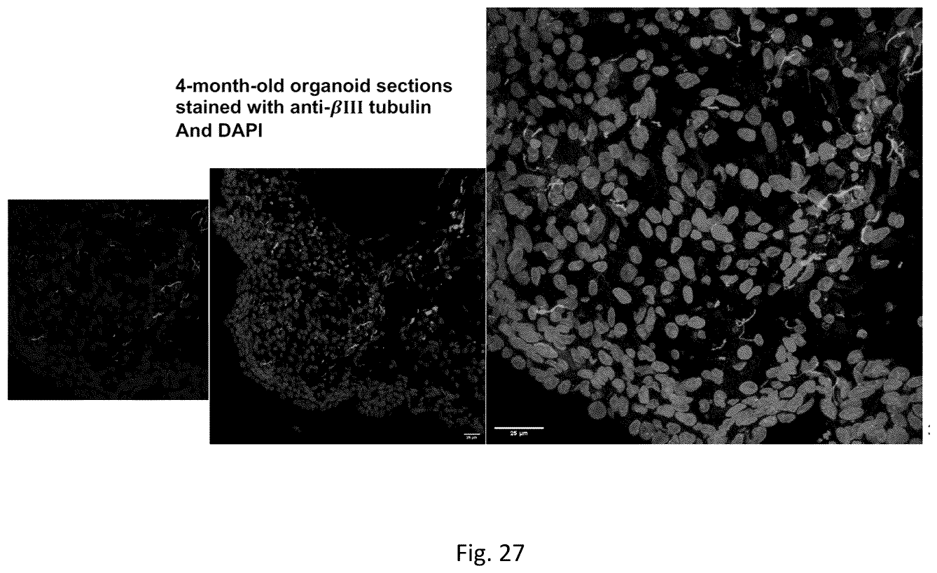

In some examples, HCOs were derived from the NCRM-1 cell line. 4-month old HCOs were stained for TUBB3 and found to have nerve-like structures (FIG. 26). 4-month old organoid sections were stained with anti-βIII tubulin and DAPI and shown to have nerve-like structures (FIG. 27).

### Example 4: Generation of iPSC-Derived Corneal Organoids for Disease Modeling

Corneal diseases are a leading cause of blindness, disproportionately affecting younger patients compared to other eye diseases (Meek K M, Knupp C. Prog Retin Eye Res. 2015 November;49:1-16. doi: 10.1016/j.preteyeres.2015.07.001. Epub 2015 Jul. 2, World report on vision. Geneva: World Health Organization; 2019. Licence: CC BY—NC-SA 3.0 IGO). Current models rely on animal models or limited human tissues, presenting ethical and practical challenges. Human corneal organoids (HCOs) provide a promising in vitro platform for disease modeling and regenerative medicine. In this Example, a robust pipeline for generating organotypic HCOs through differentiation was developed. HCOs are simple to generate and can be efficiently derived from patients with genetic diseases using induced pluripotent stem cells (iPSCs) (Takahashi K, et al., Cell. 2007 Nov. 30;131 (5): 861-72. doi: 10.1016/j.cell.2007.11.019. PMID: 18035408, Foster, J., et al. Sci Rep 7, 41286 (2017), Maiti G, et al., PNAS Nexus. 2022 Oct. 28;1 (5): pgac246). In this Example, improvements in HCO production efficiency was demonstrated, and the application of HCOs in genetic disease modeling was highlighted.

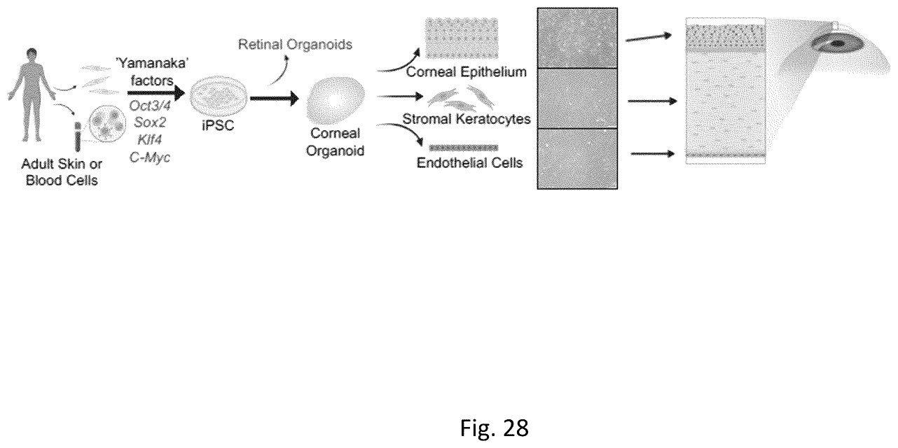

FIG. 28 depicts a schematic of the generation of iPSC-derived human corneal organoids (HCOs) from donor or patient cells.

The methods are now described.

### HCO Culture

H9 embryonic stem cells or IMR90.4 iPSCs and NCRM-1 iPSCs (derived from fibroblasts and CD34+ cord blood cells, respectively) were differentiated as described previously (Foster, J., et al. Sci Rep 7, 41286 (2017)). Initially they were maintained in mTeSR™1 on Matrigel®, passaged to single-cell density, and seeded at 3,000 cells per well in a 96-well plate before transitioning from mTeSR™ M1 with blebbistatin in hypoxia to Neural Induction Medium (NIM), followed by treatment with varying concentrations of Smoothened agonist (SAG) from Day 10-Day 20, subsequent transition to Long Term Retinal Maturation Media (LTR) (containing FBS or knockout serum replacement (KSR) at Day 15 (and BMP-4 from Day 15-Day 20 with KSR) and long-term culture in ultra-low binding plates with regular feeding, while ensuring differentiation and survival through the addition of 500 nM all-trans retinoic acid (ATRA) from Day 20-Day 120 under standard incubation conditions.

### BLIMP1/PRDM1 Model

Exon 3 of the PRDM1 gene was removed using CRISPR/Cas9 resulting in the deletion of the beta isoform of the gene and confirmed by genomic and RNA sequencing (Yen Yu Chang, et al Invest. Ophthalmol. Vis. Sci. 2023; 64 (8): 4494).

### RNA Sequencing

RNA was isolated from wild-type and knockout HCOs using the Qiagen RNAEasy Mini Kit according to manufacturer's instructions and sequenced and analyzed.

iPSC Generation

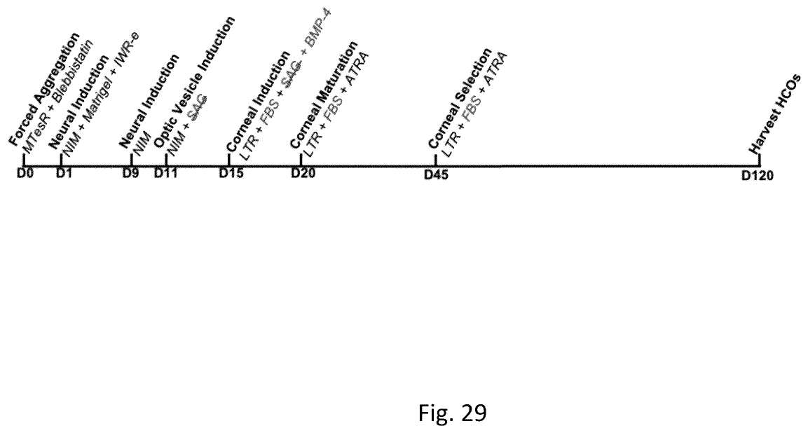

Peripheral blood mononuclear cells from keratoconus patients were obtained and subsequently processed. iPSC clones were generated using the Cytotune 2.0 Sendai Virus Reprogramming kit (ThermoFisher) according to the manufacturer's instructions. FIG. 29 depicts a schematic of a timeline for the differentiation of PSCs to HCOs with key signaling molecules and changes highlighted in red. iPSCs were harvested and aggregated, prior to induction to a neural ectoderm lineage, using NIM, Matrigel® and Wnt inhibition (IWRe). At day (D) 11, cells were induced to an early eye primordium through innate signaling and exogenous Sonic Hedgehog signaling using SAG. Cells were then switched to FBS-containing media before being maintained in LTR with FBS and ATRA for cell survival.

Results are now described.

HCOs were Successfully Differentiated from Multiple Cell Lines.

iPSC differentiation protocols can be difficult to replicate across various iPSC lines. This is due to the complex genetic/epigenetic backgrounds of different iPSC lines. In this Example, four pluripotent stem cell lines were differentiated from CD34+ blood cells, fibroblast and embryonic stem cell sources.

FIG. 7 depicts Representative images of HCOs from both IMR90.4 and NCRM-1 iPS cell lines at different stages of development, shown at day 0, 11, 36 and 111. Scale bar represents 500 μm. Co, Re and RPE indicate presumptive cornea, retina and retinal pigment epithelial, respectively.

### Decreased Sonic Hedgehog (Shh) Signaling by Removal of Smoothened Agonist (SAG) Greatly Improved the Efficiency of Corneal Organoid Production.

During human eye development, precise regulation of Shh signaling is essential for proper specification of ocular tissues (Cavodeassi F, et al., Hum Genet. 2019 September;138 (8-9): 917-936). While Shh plays a crucial role in early eye patterning, excessive activation can disrupt normal lineage commitment. Indeed, in utero, the cornea would receive reduced Shh signaling compared to the retina (Zhao L, et al., Mech Dev. 2010 January-Feb; 127 (1-2): 62-72).

In this Example, it was demonstrated that removing the Smoothened agonist (SAG) during differentiation significantly enhances corneal organoid production. By aligning organoid protocols with human developmental principles, the efficiency and reproducibility of corneal organoid formation was improved.

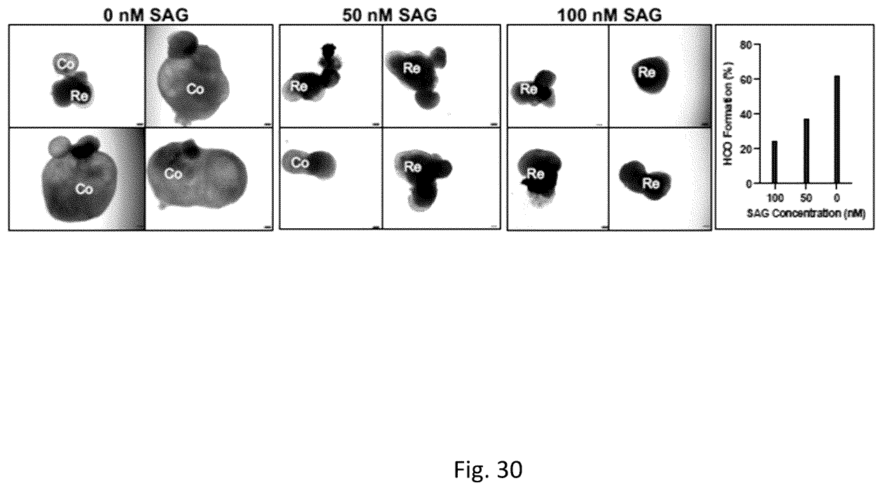

FIG. 30 depicts Representative images of HCOs from the NCRM-1 iPS cell line grown in different concentrations of Smoothened agonist (SAG). Corneal (Co) and retinal (Re) regions of organoids are indicated in the images. Decreasing SAG concentration to 0 increased corneal organoid formation from 25 to 62.5% allowing much higher throughput. Scale bars represent 200 μm.

### Elimination of Fetal Bovine Serum (FBS) Did not Affect HCO Development and Yielded Consistent, Xeno-Free HCOs.

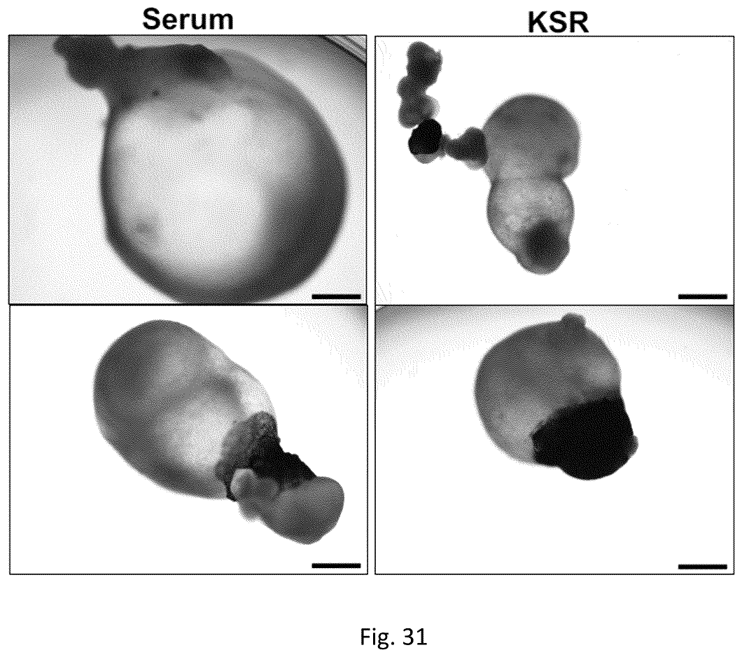

FBS in HCO cultures introduces variability due to its undefined composition and batch effects. Replacing FBS with knockout serum replacement (KSR) and BMP-4 stimulation creates a more controlled, xeno-free system that enhances reproducibility and scalability. KSR also enables precise interrogation of individual growth factors that influence HCO development, allowing for the dissection of their roles in corneal lineage specification and disease pathogenesis. This approach improves the consistency of organoid generation while making the system more suitable for translational and clinical applications. FIG. 31 depicts a human corneal organoids (HCO) grown in fetal bovine serum and an HCO grown in Knockout Serum Replacement-containing media (KSR) at 3 months of culture. Scale bar is 1000 μm.

BLIMP1a Mutant HCOs Exhibited Disrupted Epithelium and Downregulation of Multiple Keratins Highlighting their Potential as Genetic Disease Models.

B lymphocyte-induced maturation protein 1 (BLIMP-1 or PRDM1 gene) is a transcription factor. BLIMP-1 regulates many other pathways through its repression of c-Myc, including developmental pathways (Vincent S D, et al., Development. 2005 Mar; 132 (6): 1315-25). BLIMP-1 is implicated in skin epithelial homeostasis and significant epithelial disruption is observed in conditional BLIMP-1 knockouts and full ablation leads to prenatal death. It has been observed in presumptive corneal epithelial stem cells and more recently, in a Drosophila model, BLIMP-1 ablation leads to significant corneal lens defects and so it was sought, in this Example, to better understand the human disease relevance using HCOs (Chiang M F, et al., Proc Natl Acad Sci USA. 2013 Apr. 16;110 (16): 6476-81, Wang H, et al., Development. 2022 Apr. 1;149 (7): dev200217.).

BLIMP1a isoform mutant Embryonic Stem(ES) cells were used and were able to differentiate into HCOs, but the corneal epithelium was disrupted and poorly stratified compared to organoids derived from wild-type cells. RNA-seq data showed downregulation of several keratin genes including KRT4, KRT13, KRT15, KRT17, and KRT40. Other corneal-relevant genes, including the ECM genes COL9A1, FMOD and COL9A1 and stem cell marker genes, CEBPD, NPNT and MELTF were also differentially expressed. These experiments are repeated with more samples to better understand the signaling pathways affected and to understand the role BLIMP1 plays in corneal epithelial homeostasis.

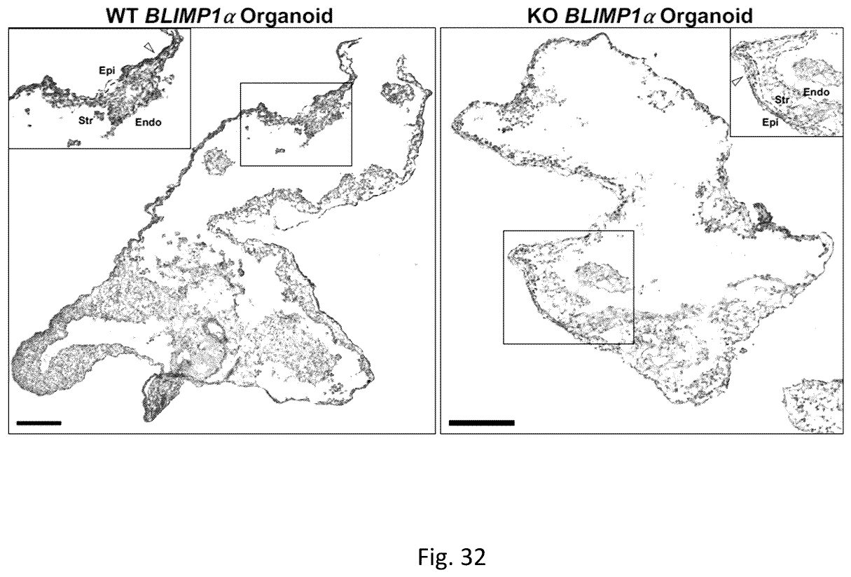

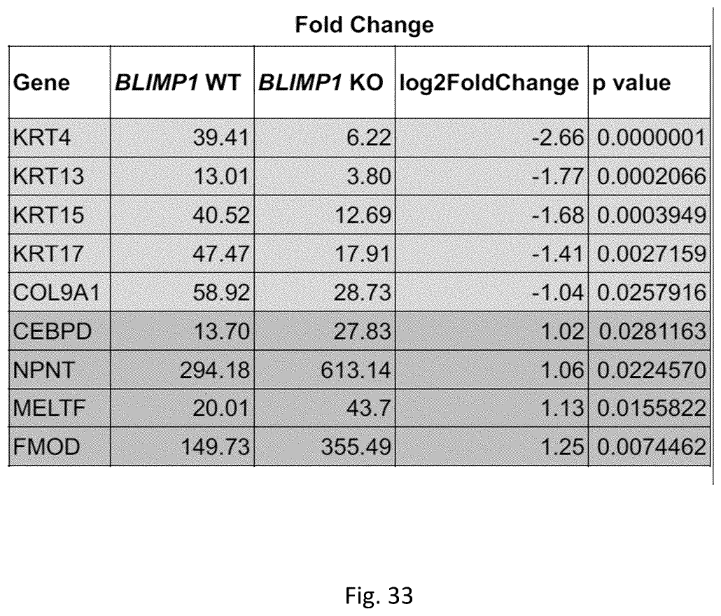

FIG. 32 depicts BLIMP1 a wildtype (WT) and knockout (KO) two-month old corneal organoids stained with hematoxylin and eosin. Presumptive corneal stroma-like regions are indicated in boxes and shown as insets. The three principal corneal stromal-like layers are indicated in the images-epithelium (Epi), stroma (Str) and endothelium (Endo). Arrowheads in the images indicate corneal epithelial-like cells, which are well stratified in WT but appear disrupted in KO, including outside of cornea-like areas. Scale bars represent 200 μm. FIG. 33 depicts selected gene normalized values (counts per million-CPM) with altered expression in BLIMP1a knockout corneal organoids. Downregulated genes are highlighted in orange and upregulated genes in green.

Generation of Patient-Specific iPS Cell Lines from Keratoconus Patients Yields New Insights into Disease Pathogenesis.

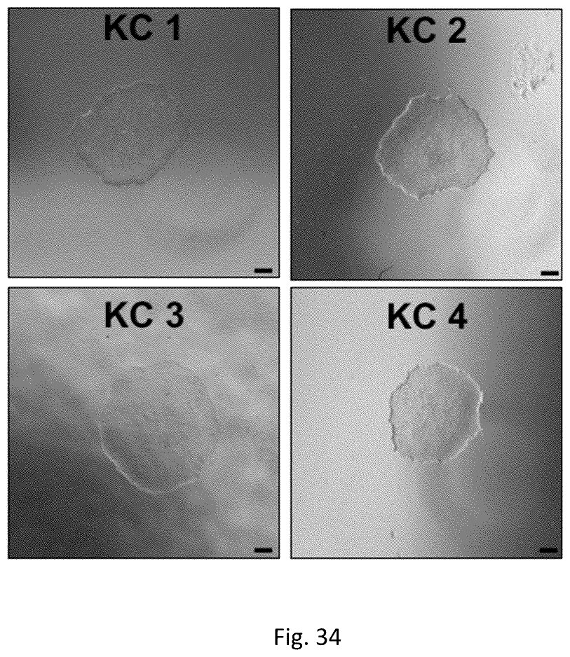

The genetic basis of keratoconus in a controlled, disease-relevant model using patient-derived iPSCs. Whole-genome or exome sequencing of these cell lines allows for the identification of candidate pathogenic variants that may contribute to disease progression. By testing these variants in vitro-through gene editing or pathway analysis in corneal organoids-their functional impact on cell behavior and extracellular matrix integrity can be determined. This approach provides a direct way to link patient's genetic changes to disease mechanisms, offering new insights into keratoconus disease. Table 1 shows the disease severity of keratoconus patient samples that have been transduced to iPSCs (Table 1, Below). FIG. 34 depicts representative iPSC colonies derived from the 4 patients of Table 1.

TABLE 1

| | | | | | Keratoconus |
|---|---|---|---|---|---|
| | | | | | Severity |
| | | | | | (Maximum |
| | | | | | Keratometry/K |
| | | | Pentacam | Pentacam | Max): <45 = |
| | | | Data - Left | Data - Right | mild, >45 |
| | | | Eye - Cornea | Eye - Cornea | but <52 = |
| Keratoconus | | Keratoconus | thickness (in | thickness (in | moderate, >52 = |
| Status | Age | Laterality | μm) | μm) | severe) |
| Patient (1) | 47.8 | Both | 521 | 480 | Severe |
| Patient (2) | 32.1 | Both | 493 | 560 | Severe |
| Patient (3) | 27.3 | Both Eyes | 440 | 461 | Moderate |
| Patient (4) | 26.3 | Left Eye | 440 | 479 | Moderate |
| Control | 70.7 | None | N/A | N/A | None |

A summary of the example is now described.

HCOs were generated from multiple iPSC lines, demonstrating a reliable and reproducible method. Removing Sonic Hedgehog (Shh) signaling via Smoothened agonist (SAG) significantly improved corneal organoid formation efficiency. Eliminating fetal bovine serum did not affect HCO development, enabling consistent, clinically relevant organoids. Patient-specific iPSC-derived HCOs provide new insights into keratoconus pathogenesis and potential therapeutic targets. BLIMP1a mutant HCOs exhibited disrupted corneal epithelium and downregulation of multiple keratins, highlighting their utility for studying a variety of genetic corneal disorders.

### Example 5: Human Corneal Organoid (HCO) Modified Protocol

An exemplary protocol for the generation of HCOs is now described.

The materials for the protocol are now listed.

MTesR (Stem Cell, Cat no. 85850)

iPSC lines (IMR 90.4 and NCRM-1)

Matrigel® (Corning, Cat no. 354230)-dilute one aliquot in DMEM

Cell Dissociation Buffer (Thermo, Cat no. 13151014)

Dulbecco's Phosphate-Buffered Saline (DPBS), without calcium and magnesium (Thermo, Cat no. 14190144)

Hank's Balanced Salt Solution (HBSS) (Thermo, Cat no. 14025076) 10 mM solution of all-trans-retinoic acid (ATRA). 10 mM ATRA solution may be formed by diluting 50 mg ATRA in 16 ml of dimethyl sulfoxide (DMSO). 10 mM ATRA solution may be filter sterilized. 10 mM ATRA solution may be aliquoted and stored at −80 °C

10 mM (−)-Blebbistatin solution. 10 mM (−)-Blebbistatin solution may be formed by diluting 1 mg (−)-Blebbistatin in 340 μL DMSO. 10 mM (−)-Blebbistatin solution may filter sterilized and may be stored at −80 °C.

E6 supplement. E6 supplement may comprise 97 mg NaHCO₃, 97 mg insulin, 53.5 mg holo-transferrin, 320 mg L-ascorbic acid, and 70 μg sodium selenite diluted in 100 mL water. E6 supplement may be aliquoted in 10 mL aliquots. Aliquots may be stored in a 15 mL tube. Aliquots may be stored at −80 °C.

3 mM IWR-1-endo solution. 3 mM IWR-1-endo solution may be formed by diluting 10 mg IWR-1-endo in 9.1 mL DMSO. 3 mM IWR-1-endo solution may be filter sterilized. 3 mM IWR-1-endo solution may be aliquoted in 50 μL aliquots and may be stored at −80 °C.

400 mM taurine solution. 400 mM taurine solution may be formed by diluting 500 mg taurine in 10 mL water. 400 mM taurine solution may be aliquoted in 1.25 mL aliquots and may be stored at −80 °C.

Neural induction medium (NIM). NIM may comprise 1% B27 (without vitamin A), 1 mM pyruvate, 1× non-essential amino acids, 1× GlutaMAX™, 2× E6 supplement, and 0.44 g NaCl in 455 mL Dulbecco's Modified Eagle's Medium (DMEM). NIM may be formed by combining 455 mL DMEM, 10 mL B27 stock solution, 5 mL pyruvate stock solution, 5 mL non-essential amino acids stock solution, 5 mL GlutaMAX™ stock solution, and two 10 mL E6 supplement aliquots, and then diluting 0.44 g NaCl in the combined solution.

Long-term maturation medium (LTR). LTR may be made with serum or may be serum-free. LTR with serum may comprise DMEM and F12 in a 3:1 ratio, 1% B27 supplement, 10% heat inactivated fetal bovine serum (HI-FBS), 1 mM pyruvate, 1× non-essential amino acids, 1× GlutaMAX™, 1 mM taurine, 1× Penicillin-Streptomycin (Pen/Strep), and 500 nM ATRA. The ATRA may be added from an ATRA stock solution shortly before the media is used. Serum-free LTR may be made by replacing the HI-FBS of LTR with serum with an equal amount of KSR and keeping the other components the same. LTR with serum may be formed by combining 324 mL DMEM, 108 mL F12, 10 mL B-27™ supplement, 45 mL HI-FBS, 5 mL pyruvate stock solution, 5 mL 100× non-essential amino acids, 5 mL 100× GlutaMAX™, a 1.25 mL taurine solution aliquot, 5 mL 100× Pen/Strep, and 1 μL ATRA stock solution per each 20 mL LTR. The ATRA solution may be added shortly before the media is used. Serum-free LTR may be formed similarly, except 45 mL KSR may be substituted for 45 mL HI-FBS.

Steps of the protocol are now described.

Forced Aggregation of iPSCs

iPSCs are passaged by any method. During iPSC passaging, blebbistatin is added to a solution that is used to wash the iPSCs. For example, iPSCs may be washed in a cell medium or buffer containing 5 μM blebbistatin. iPSCs are then resuspended in a suitable iPSC cell culture medium containing blebbistatin, for example mTesR™ containing 5 μM blebbistatin. iPSCs may be resuspended at a concentration of 60,000 cells/mL. 50 μL of the iPSC suspension is added to each well of a 96 well plate using a microchannel pipette such that 3,000 cells are added to each well. The 96 well plate is centrifuged at 400 g for 5 minutes such that the cells are collected at the bottom of the wells. The 96 well plate is placed in a hypoxic environment overnight to promote cell survival and aggregation.

B-27™ supplement, FBS, HI-FBS, or KSR may be added to appropriate media shortly before the media is used rather than being prepared in advance.

### Neural Induction

On Day 2 forced aggregates are induced to a neuronal phenotype by adding 50 μl of NIM, supplemented with 2% Matrigel® and 6 μM IWR1-e to each well of the 96 well plate. This solution can be made by adding one tube of Matrigel® and 10 μL of IWR1-e stock to 5 mL of NIM.

The cells are then transferred to a normoxic environment. For example, the 96 well plate may be transferred to a standard cell culture incubator.

On days 3 and 4, the forced aggregates are fed daily by adding 50 μl of NIM, supplemented with 1% Matrigel® and 3 μM IWR1-e to each well of the 96 well plate. This solution can be made by adding 3 tubes of Matrigel® and 30 μL of IWR1-e stock to 30 mL of NIM.

On Days 5, 6, and 7 forced aggregates are fed daily with a 50% media change of each well (100 μL). The media change is done with NIM supplemented with 1% Matrigel® and 3 μM IWR1-e.

On Day 9, forced aggregates are fed with 100 μL of NIM. Media is changed every other day beginning on Day 9.

On Day 11, forced aggregates are pooled into 15 mL tubes by washing the forced aggregates out with widened 200 μL pipettes and placing them into a dish. The forced aggregates are collected and pooled into a 15 mL tube and washed three times with HBSS. The forced aggregates are now referred to as organoids. Pooled organoids are resuspended in 10 mL NIM and plated one organoid/well in a 24-well ultra-low attachment plate.

### Neural Vesicle Excision

On day 13, the organoids are fed with 500 μL NIM.

### Organoid Maturation and Selection

On day 15, the media is switch to LTR. This may be serum-free LTR or LTR with serum. If serum-free LTR is used, the protocol continues using only serum-free LTR. If LTR with serum is used, the protocol continues using only LTR with serum. Media is then changed on Days 17 and 19. If serum-free LTR is used, 20 ng/ml BMP-4 (1.5 nM BMP-4) may be added to the serum-free LTR.

On day 21, organoids (vesicles) are fed with LTR that includes ATRA every 2-3 days. ATRA is added fresh to the LTR on the day of feeding. 2.5 μL of ATRA stock solution may be added to 50 mL of LTR to yield LTR with 500 nM ATRA.

### Example 6: Laminin Coating, Expansion of HCO-Derived Cells, and Enrichment of HCO-Derived Cell Cultures for Stromal Cells or Epithelial Cells

An exemplary protocol for laminin coating and expansion of HCO-derived cells is now described.

The materials for the protocol are now listed.
iMatrix-221 (Amsbio, AMS.892-061).
iMatrix-511 (Amsbio, AMS.892-011).
DMEM: F12.
50 mM ascorbic acid solution. 50 mM ascorbic acid solution may be stored as aliquots.

Steps of the protocol are now described.

Coating Wells with iMatrix-221 and iMatrix-511

Matrix solution was prepared by diluting 19.2 μL iMatrix in 4 mL of phosphate-buffered saline without calcium and Magnesium (PBS (−)). 2 mL of matrix solution was dispensed into each well of a tissue culture plate. The wells of the tissue culture plate were incubated either for 1 hour at 37 °C, 3 hours at room temperature, or overnight at 4 °C.

### Organoid Digestion

Organoids were incubated in ACCUMAX™+400 μg/mL DNase I for 1 hour with occasional trituration. Cell suspension was filtered through a 70 μM cell strainer. Cell suspension was centrifuged to form a pellet. The pellet was then resuspended in DMEM: F12. The cells were then counted.

### Cell Seeding

The cell suspension was split into two equal parts. The first portion was applied to a coated well and incubated for 20 minutes. Remove the cell suspension from the well. Note that some cells remain attached to the well. Wash the well twice with 1 mL DMEM: F12. Replace the media in the well with keratocyte growth media (5% FBS in DMEM: F12+L-ascorbic acid). 50 μL of the cell suspension removed from the well was used for cell counting. The process is then repeated for the second portion of the initial cell suspension. The remaining cells were then resuspended in keratocyte media (5% FBS), and replated in an uncoated well with 5% FBS. The cells that adhere to the coated well were enriched for epithelial cells. The cells that remained in suspension after washing the laminin coated wells were enriched for stromal cells.

### Example 7: Human Corneal Organoids for modeling infection

Herpes simplex virus infection is a common cause of viral keratitis in humans. In this Example, HCOs were used to model infection. The results of this example show that HCOs are a useful model for use in an infection platform. HCOs may be used in an infection platform as an alternative to animal models.

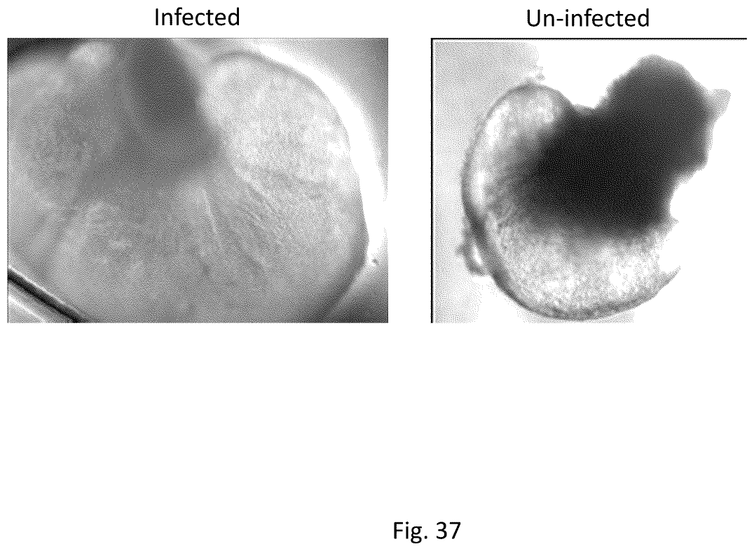

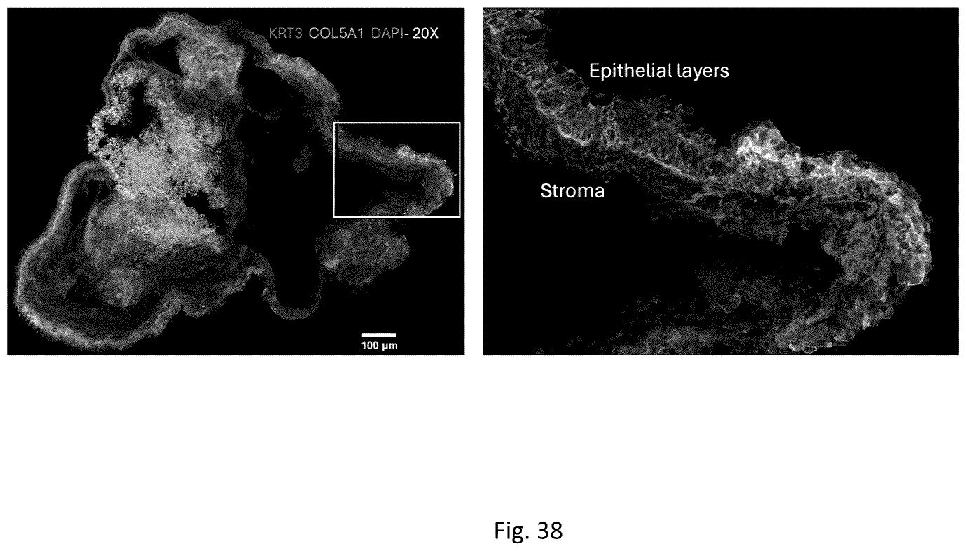

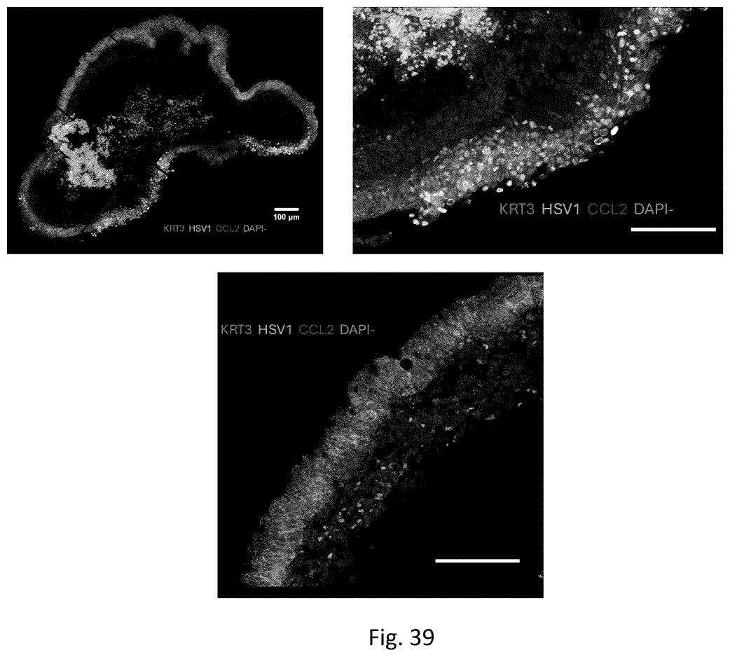

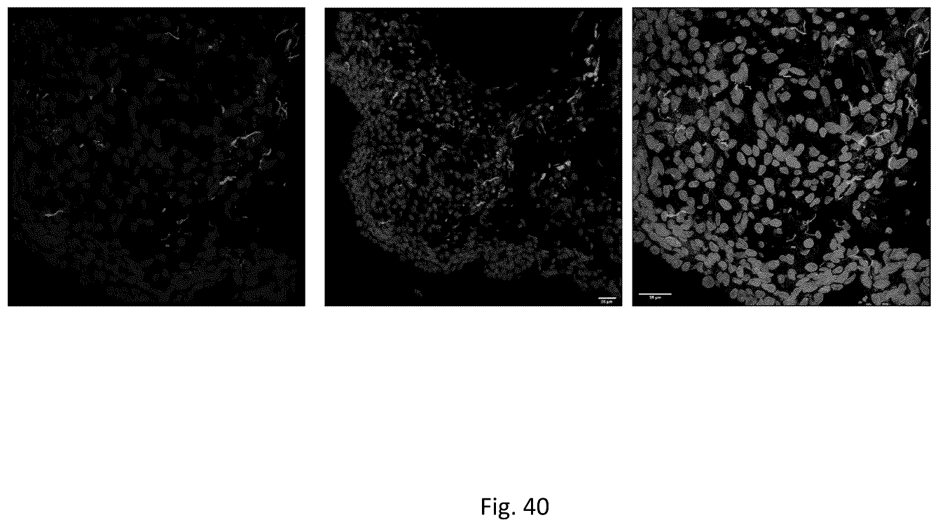

FIG. 37 depicts representative images of infected and un-infected HCOs. HCOs were infected by incubation with HSV1-mCherry (KOS) at a concentration of 103 pfu/organoid in a 24-well plate for 60 minutes at 37 °C. HCOs were washed three times in PBS and co-cultured with 50,000 human PBMCs for 24 hours at 37 °C. HCOs were than prepared for analysis. HCOs were washed, fixed, embedded in OCT, and section. Immunohistochemistry was then performed on the sections (FIG. 38-FIG. 40). HCOs were stained for KRT3 and COL5A1 (FIG. 38). HCOs were also stained for KRT3, CCL2, and HSV-1 detected by mCherry fluorescence (FIG. 39). HCOs were also immunostained for TUB-III to visualize nerve-like structures (FIG. 40).

### Example 8: Human Corneal Organoids for High-Throughput Screens

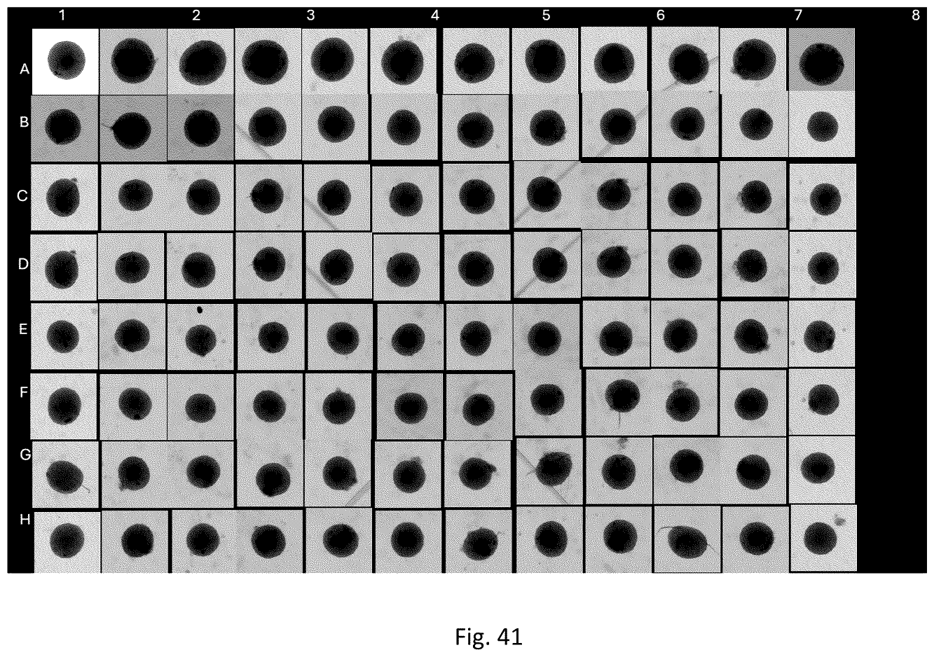

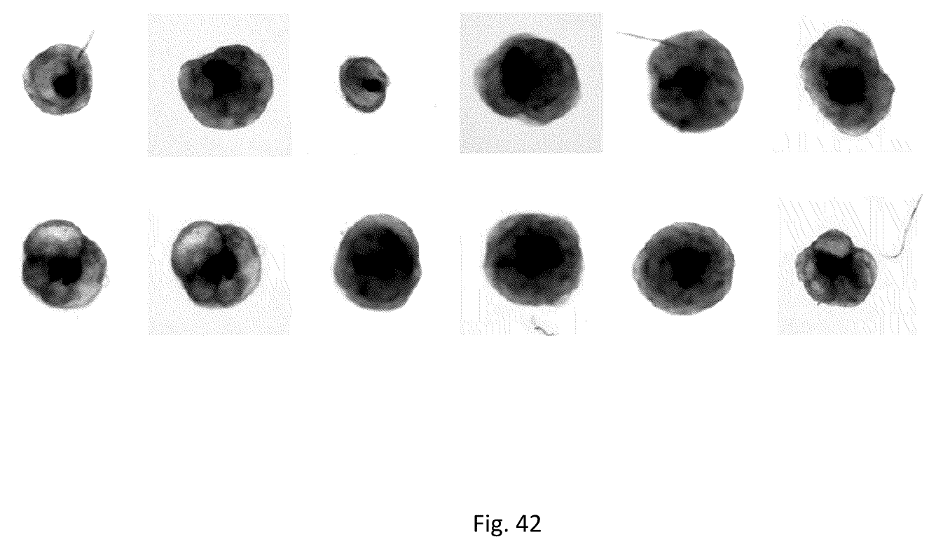

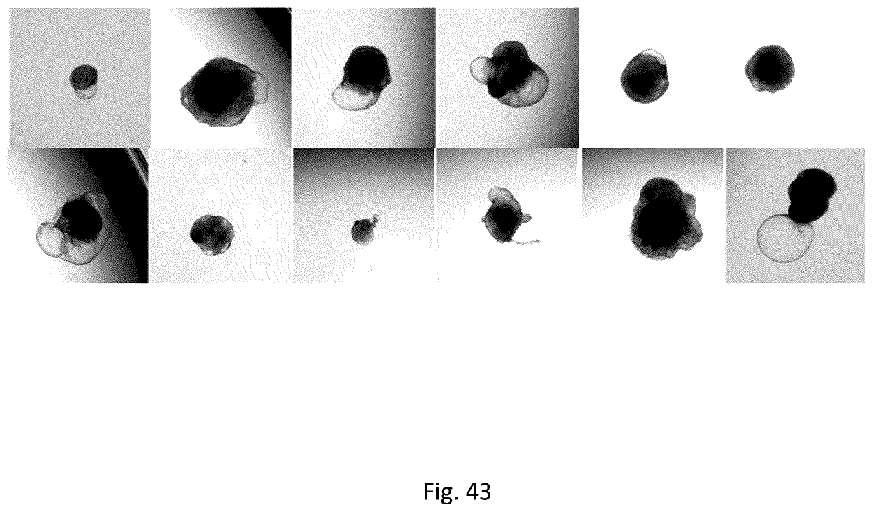

The results of this example show that Human Corneal Organoids (HCOs) are a useful model for high-throughput screens. HCOs derived from a NCRM-1 induced pluripotent stem cell (iPSC) line were cultured in wells of a 96 well plate (FIG. 41). HCOs were cultured and matured in the 96 well plate (FIG. 42 and FIG. 43).

### Example 9: GPX3 as a Severity Marker in Keratoconus

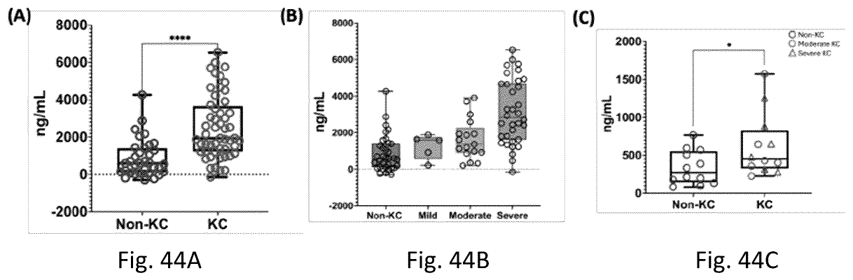

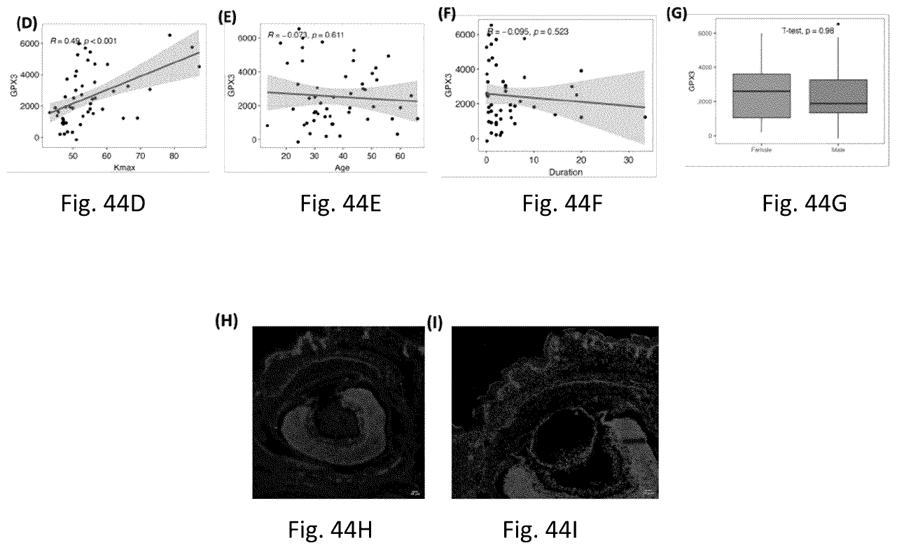

Glutathione peroxidase 3 (GPX3) is a critical extracellular antioxidant enzyme that catalyzes the reduction of hydrogen peroxide, thereby protecting tissues from oxidative injury. GPX3 has previously been studied in systemic conditions and various ocular contexts, but its role in tear fluid and specifically in KC remains underexplored. Recent investigations have demonstrated a significant upregulation of GPX3 in the tear fluid of KC patients, as measured using capillary-based collection methods (FIG. 44A). Interestingly, this finding contrasts with prior transcriptomic and proteomic observations in KC corneal tissue, where GPX3 levels were found to be reduced (Shinde et al., OMICS, 2019; Shinde et al., Sci Rep. 2020; 10 (1): 9907) This disparity points to a potentially compensatory, localized antioxidant response in the tear film. To further investigate the source of GPX3 in the tear film, the eyes of postnatal day 0 (P0) mice were examined, and prominent GPX3 expression in the epithelial tissues was observed (FIG. 44I). This suggests that the GPX3 detected in tear fluid may be secreted by the conjunctival epithelium, which is a major contributor to tear composition, given its larger surface area on the ocular surface. To validate this hypothesis, Future analysis will be conducted on conjunctival epithelial cells collected through impression cytology from patients. Thus far, GPX3 upregulation in the tear fluid of KC patients has been shown and positively correlates with the severity of the disease (FIG. 44D).

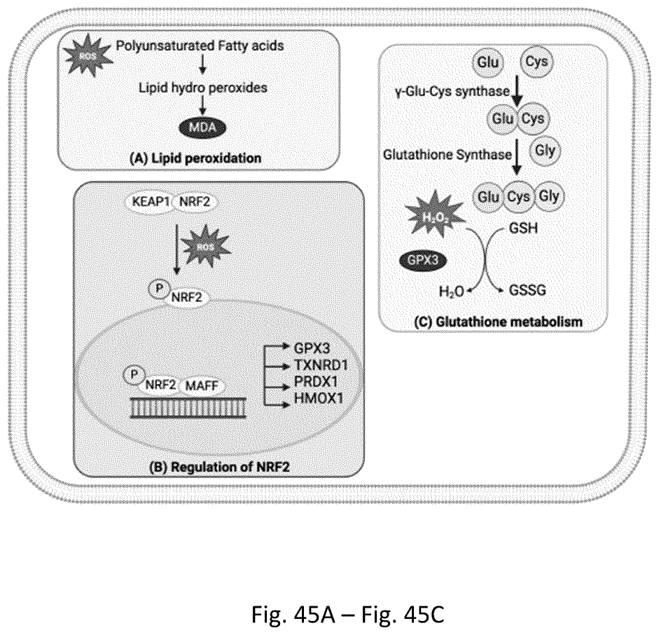

In fact, GPX activity was previously analyzed in KC patients by Balmus et al. (Balmus et al., Exp Eye Res. 2020; 190:107897). However, there are eight types of glutathione peroxidases (GPXs) (Brigelius-Flohe and Maiorino, Biochem Biophys Acta. 2013; 1830 (5): 3289-3303), and their study did not specify which isoform was measured. In contrast, GPX3 was chosen due to preliminary transcriptomic and proteomic analyses revealing dysregulation in the NRF2 signaling pathway (FIG. 45), which is known to play a critical role in oxidative stress regulation in KC (Shinde et al., OMICS, 2019; Shinde et al., Sci Rep. 2020; 10 (1): 9907; Monteiro de Barros and Chakravarti, Exp Eye Res. 2022). In addition, the integrated stress response in KC has been explored (Foster et al., Invest Ophthalmol Vis Sci. 2018; 59 (7): 2977-2986). Future research will focus on investigating NRF2 signaling more closely by establishing in vitro models. As part of this investigation, whether any NRF2 target proteins are present in the tear film was tested, which may help in diagnosis of KC. GPX3 and malondialdehyde (MDA-a marker of lipid peroxidation) were selected for analysis. Among these, GPX3 is positively correlated with disease severity in KC patients. Notably, previous studies have neither discussed nor identified the source or tear fluid levels of GPX3.

The finding that the protein GPX3 increases in tear fluid samples of keratoconus patients establishes a useful biomarker for either early detection of keratoconus or tracking progression of disease. GPX3 also shows an increase in the serum of patients (FIG. 44C).

### Example 10: Deciphering NRF2-Dysregulations and Oxidative Stress in Keratoconus Corneal Degeneration

Keratoconus (KC) is a corneal disease where the stroma shows progressive degenerative thinning. Previous studies have shown downregulation of NRF2-regualted antioxidant genes in patients with KC. The objective herein is to test specific NRF2-target genes, track oxidative damage byproducts in KC patients, and develop a cell culture model of oxidative stress.

Patients were included if they have a keratoconus diagnosis, but not syndromic keratoconus (E.g. Down syndrome). Healthy controls were included if they do not have a keratoconus diagnosis and do not have eye redness or inflammation. Tears were collected by a capillary tube method, and ELISA was run to determine the amount of GPX3 (in 15 non-KC samples and 21 KC samples), and MDA (in 16 non-KC samples and 19 KC samples. Epithelial scrapings were collected for qPCR of NRF2 target genes in 4 KC samples and 4 DN samples. The NRF2 targets were compared between the severity, age, gender, and duration of disease. In vitro validation was performed by the extraction of corneal fibroblasts (in 2 KC and 2 DN samples) and induction of oxidative stress using 100 μM H₂O₂.

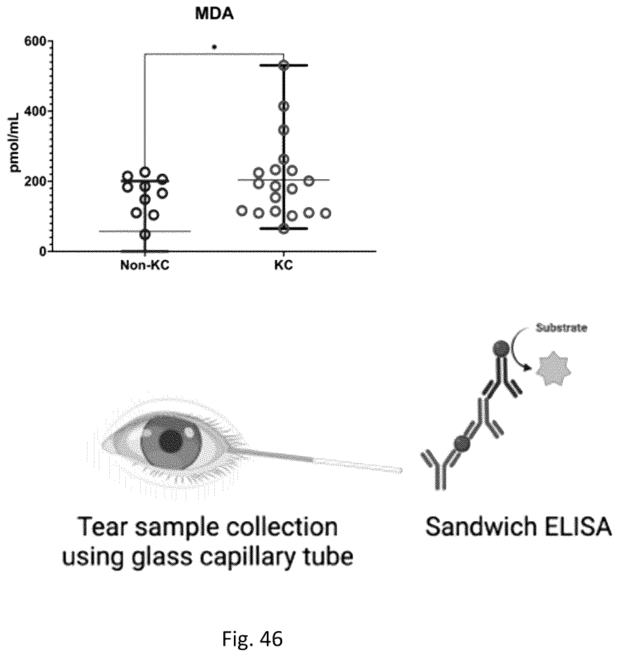

Immunofluorescence for KRF2 and MAFF, qRT-PCT for NRF2 target genes, and cell viability-resazurin assay for metabolic activity were all conducted. MDA levels are significantly elevated in KC tears (FIG. 46). KC tears were collected using a glass capillary tube and MDA levels were assayed using a sandwich ELISA. In the tear samples of donor normal (DN), MDA levels were found to be 57.58+/−142.91 (mean±/−SD) pmol/mL, while in KC tears, MDA levels were elevated to 204.36+/−118.37 μmol/mL (p<0.011).

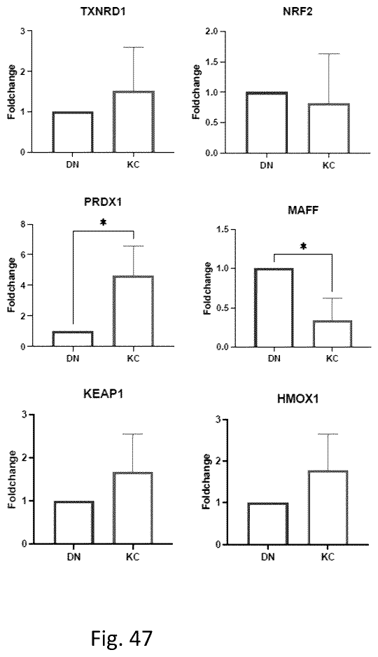

Gene expression relative to GAPDH measured by Taqman qRT-PCR in total RNA from epithelial scraping of DN and KC (n=4) was performed (FIG. 47). Peroxiredoxin I (PRDX1) is significantly upregulated (p<0.033) in KC, whereas MAF BZIP Transcription Factor F (MAFF) was decreased (p<0.0172). Expression of the other genes tested was not significantly different.

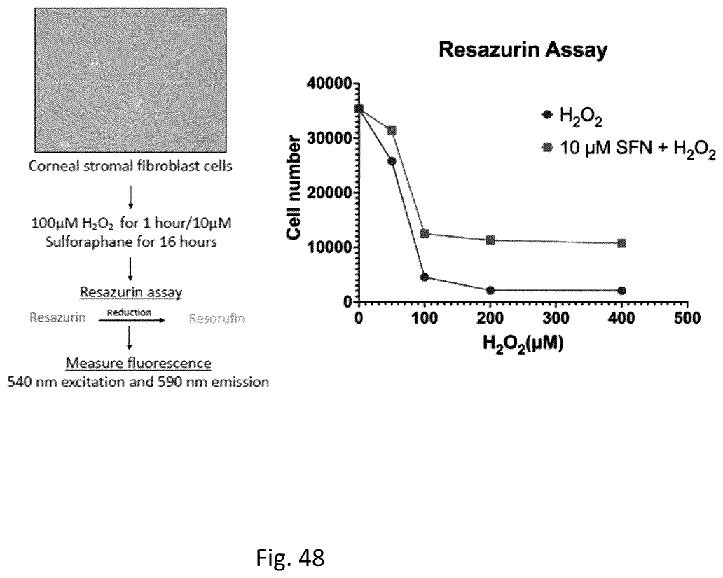

FIG. 48A depicts the experimental design of testing a cell culture model of oxidative stress and viability assay. Sulforaphane improved cell viability in fibroblast treated with hydrogen peroxide (FIG. 48B).

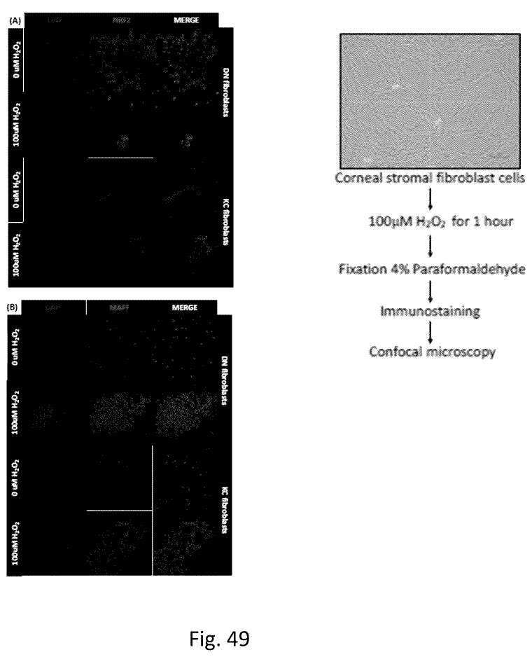

DN and KC fibroblasts were immunostained for NRF2 and MAFF. In DN fibroblasts, the intensity of NRF2 increased in response to oxidative stress, while in the KC fibroblasts, the intensity was reduced (FIG. 49). Similarly, the intensity of MAFF was lower in KC fibroblasts compared to DN fibroblasts (FIG. 49).

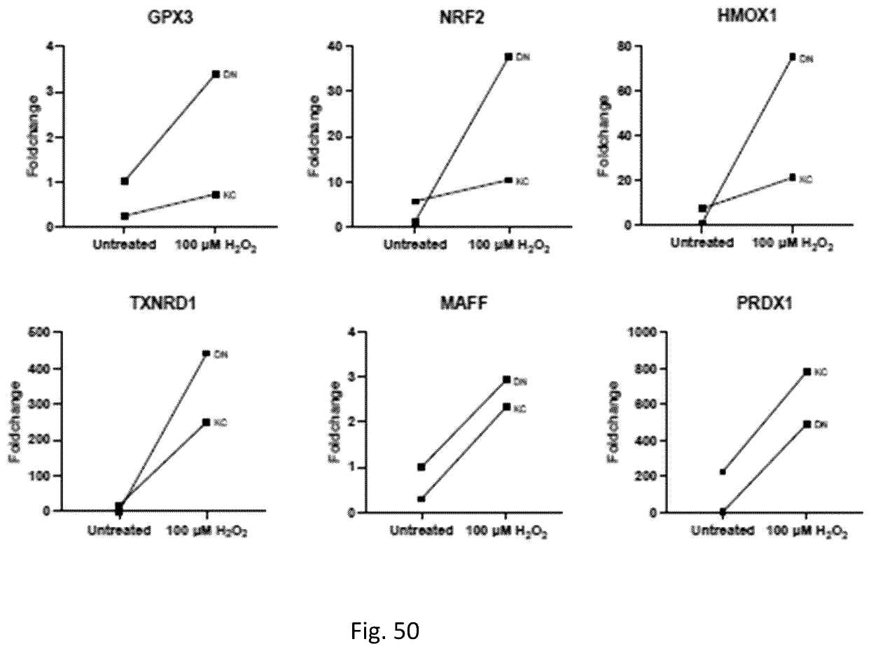

NRF2 target genes were evaluated before and after exposure to oxidative stress in DN and KC primary fibroblasts (FIG. 50). The results indicate that all target genes were upregulated after stress. However, in KC cells, the upregulation is lower compared to DN, except for PRDX1.

Elevated levels of GPX3 and MDA indicate excessive oxidative stress on the KC ocular surface. These may serve as diagnostic biomarkers for KC. Analysis of mRNA levels in corneal epithelial scrapings revealed the downregulation of NRF2 and MAFF, and upregulation of PRDX1.

In vitro study showed upregulation of several NRF2 target genes in both DN and KC fibroblasts upon oxidative stress, while in KC fibroblasts this upregulation is not as high as in DN fibroblasts.

### Example 11: Sulforaphane-Enhanced NRF2 Activation and Antioxidant Response in Keratoconus Corneal Stromal Cells

Unresolved oxidative stress is thought to drive corneal tissue damage in keratoconus (KC). Earlier transcriptomic and proteomic studies identified dysregulated NRF2-driven antioxidants in KC corneas. To elucidate NRF2 signals and the therapeutic effects of sulforaphane, an oxidative stress cell culture model was developed and NRF2 signal regulations in hydrogen peroxide (H₂O₂)-stressed donor and KC corneal stromal cells, with and without sulforaphane were compared.

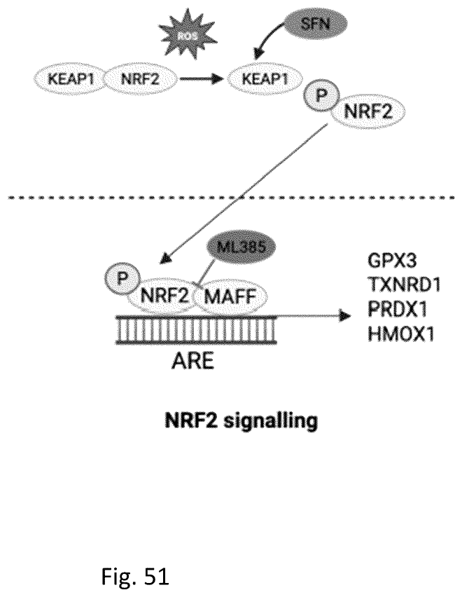

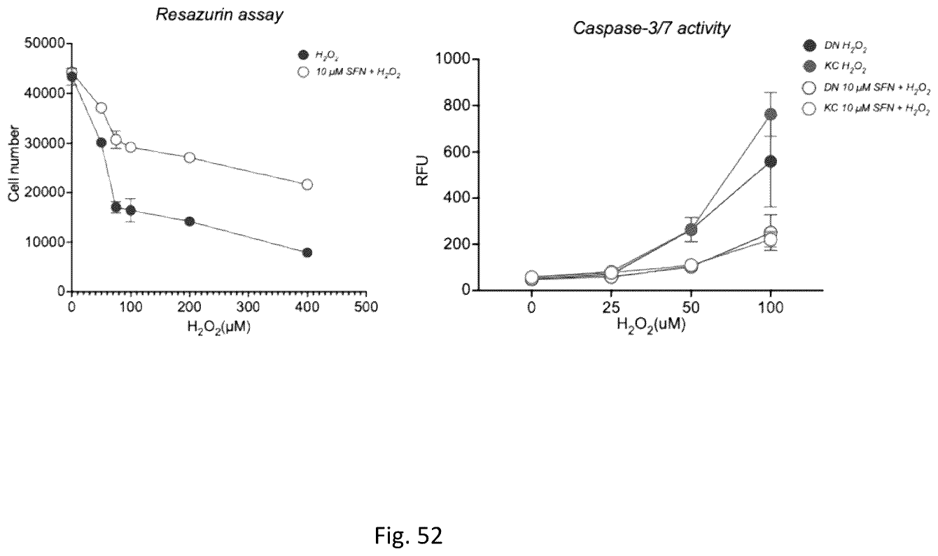

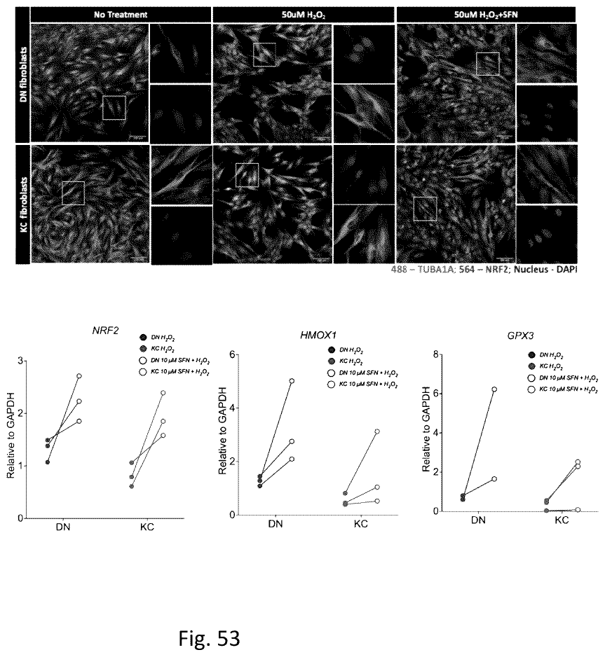

Under homeostatic conditions, NRF2 is sequestered by KEAP1 and targeted for degradation, maintaining low antioxidant gene expression. Sulforaphane disrupts the NRF2-KEAP1 interaction, allowing NRF2 to accumulate, translocate to the nucleus, and activate antioxidant genes that protect against oxidative stress and ECM degradation, which is relevant to KC. In contrast, ML385 inhibits NRF2 by binding to its CNC-bZIP domain, preventing its interaction with MAFF and subsequent binding to AREs. This blocks downstream antioxidant gene activation, suppresses antioxidant defense, and promotes ECM breakdown, mimicking KC-like pathology. This model highlights how NRF2 modulation influences corneal fibroblast responses under oxidative stress (FIG. 51).

The corneal stromal fibroblasts used herein were derived from keratoconus patients, while cadaveric corneas served as controls. The use of patient-derived corneal stromal cells is a key strength of this study. Unlike most in vitro studies that rely on established cell lines, the inherent characteristics of the disease were preserved. Procuring diseased tissue presents several challenges, including obtaining patient consent, coordinating with clinicians, and ensuring proper sample collection and culture establishment. Therefore, the in vitro data generated represents highly valuable and rare material.

Low-passage primary cultures of KC (n=3) and donor (DN; n=3) corneal stromal fibroblasts were exposed to H₂O₂, both before and after treatment with sulforaphane. For NRF2 activation, cells were treated with 10 μM SFN for 16 hours. Oxidative stress was induced by exposure to 50 μM H₂O₂ for 1 hour. NRF2 was inhibited by treatment with 10 μM ML385 (or DMSO control) for 7 days.

Cell viability was assessed using the Alamar Blue, trypan blue, and resazurin assays in H₂O₂ (25 μM to 400 μM) and with/without 10 μM SFN. Apoptosis was determined by Caspase-3/7 activity measurement. NRF2 accumulation and nuclear translocation were assessed via immunofluorescence staining for NRF2, FN1 and COL5Alconfocal microscopy, with mean fluorescence intensity (MFI) quantified using Fiji ImageJ. NRF2-regulated antioxidant gene expression (TXNRD1, HMOX1, PRDX1, GPX3) was assayed using TaqMan, were compared as fold change (FC) in the KC and DN cells before and after treatments.

GraphPad Prism, with two-way ANOVA for grouped comparisons and Mann-Whitney U test for unpaired data (p<0.05) was used for statistical analysis. 50 μM H₂O₂ resulted in 69.52±1.24% viability, which could be increased to 83.93±0.91% with sulforaphane (FIG. 9). Nuclear NRF2 was significantly lower in KC fibroblasts (13.76±6.46 AU of MFI; p<0.001) compared to DN fibroblasts (31.35±14.69 AU) (FIG. 10). Sulforaphane increased NRF2 nuclear translocation in both KC (31.79±13.48 AU) and DN (44.49±15.6 AU) H₂O₂-stressed fibroblasts. NRF2 gene expression was significantly lower in KC fibroblasts (FC=0.45±0.14, p<0.034) compared to DN fibroblasts, but sulforaphane treatment increased NRF2 expression in KC fibroblasts (FC=1.32±0.4). TXNRD1, HMOX1, PRDX1, and GPX3 expression was lower in KC fibroblasts (FC=0.84, 0.87, 0.98, 0.23) compared to DN fibroblasts, but under oxidative stress, these genes were upregulated in KC fibroblasts (FC=14.12, 2.85, 3.47, 3.06) relative to KC basal levels.

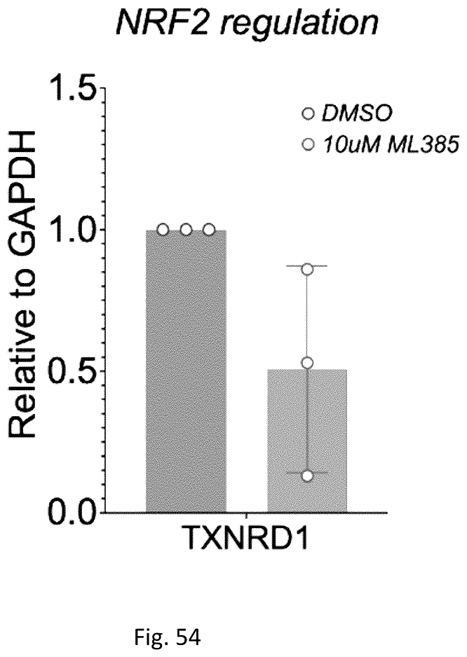

The gene expression of NRF2 downstream target genes, including TXNRD1, was assessed in corneal stromal cells treated with ML385 (an NRF2 inhibitor, FIG. 54). The results show reduced expression of these genes in ML385-treated cells compared to controls, confirming the successful inhibition of NRF2 signaling and its downstream targets.

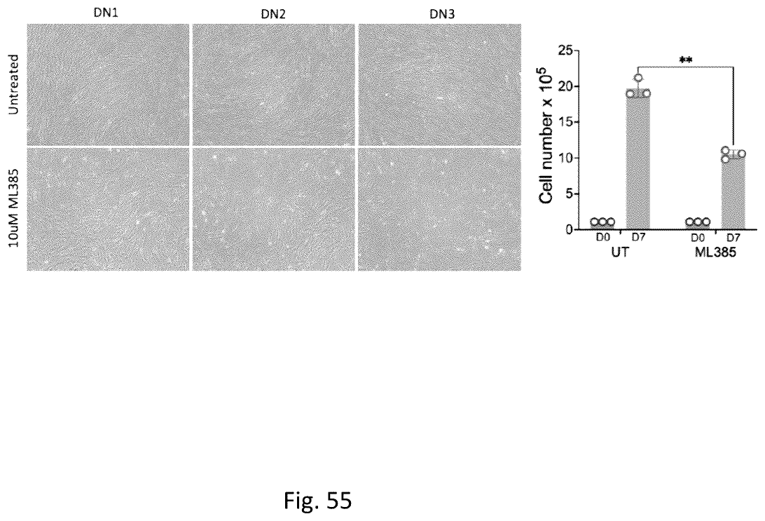

NRF2 inhibition reduces proliferation in corneal stromal cells (FIG. 55). Cell counts revealed a reduction in cell number in ML385-treated cells compared to controls, indicating decreased proliferation over the 7-day period. Future experiments assessing Ki-67 staining and propidium iodide analysis for cell cycle staining will further show cell proliferation in these conditions.

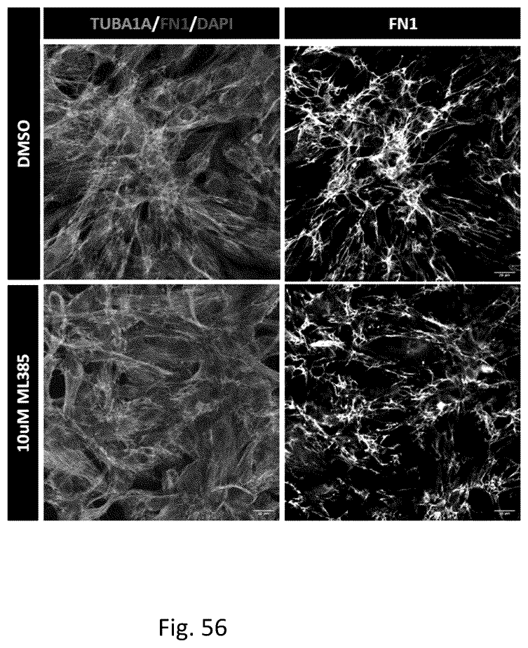

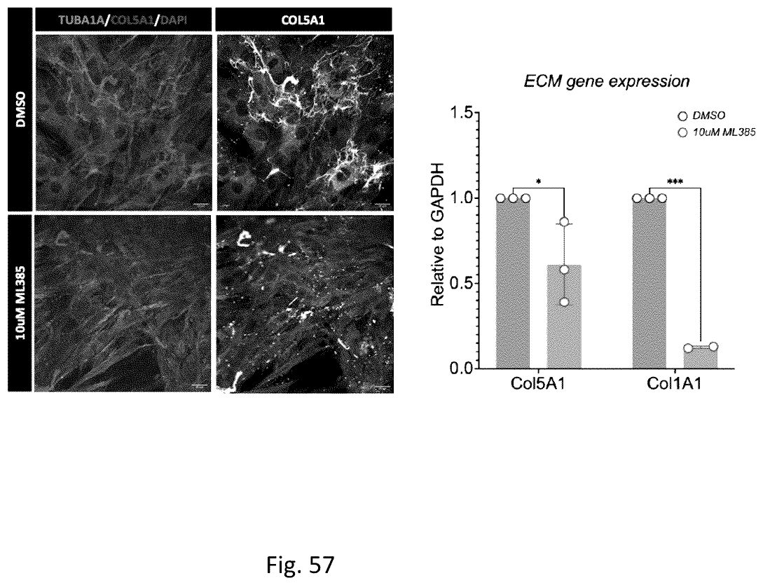

Fibronectin-1 (FIG. 56) and Col5A1 and Col1A1 (FIG. 14) are reduced after NRF2 inhibition. These findings imply that NRF2 may influence collagen synthesis, potentially through its effect on cell proliferation which disrupts ECM regulation.

SFN protects corneal fibroblasts from oxidative stress by improving cell viability and reducing apoptosis, highlighting its potential as a therapeutic agent for oxidative damage in corneal stromal cells. SFN also activates NRF2 signaling in corneal fibroblasts, enhancing the expression of NRF2-related genes and providing protective effects against oxidative stress. NRF2 inhibition via ML385 reduces cell proliferation and ECM protein expression in both donor normal (DN) and keratoconus (KC) fibroblasts, indicating NRF2's role in fibroblast proliferation and ECM production. In keratoconus, cellular damage impairs stromal cell function and leads to ECM degradation. The reduced proliferation and ECM expression in ML385-treated cells reflect similar disruptions in ECM homeostasis in KC.

SFN enhanced cell viability and NRF2 nuclear localization and gene expression in both KC and DN in H₂O₂-stressed fibroblasts. Basal NRF2 expression at mRNA and protein levels was lower in KC fibroblasts, with significantly reduced nuclear levels, but sulforaphane treatment rescued some NRF2 activity, though not to the same extent as in DN fibroblasts. Further investigation with sulforaphane treatment is ongoing.

The disclosures of each and every patent, patent application, and publication cited herein are hereby incorporated herein by reference in their entirety.

While the invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A method for forming a human cornea organoid comprising the steps of:
providing neural ectoderm lineage cells;
culturing the neural ectoderm lineage cells in an optic vesicle induction medium to induce the formation of one or more optic vesicles; and
culturing the one or more optic vesicles in a corneal induction medium comprising less than 100 nM Smoothened Agonist (SAG).

2. The method of claim 1, wherein the optic vesicle induction medium comprises less than 100 nM SAG, less than 50 nM SAG, or about 0 nM SAG.

3. The method of claim 1, wherein the corneal induction medium comprises less than 50 nM SAG or about 0 nM SAG.

4. The method of claim 1, wherein the corneal induction medium further comprises knockout serum replacement or BMP-4.

5. The method of claim 1, wherein the neural ectoderm lineage cells are cultured in the optic vesicle induction medium for about 5 days.

6. The method of claim 1, wherein the one or more optic vesicles are cultured in the corneal induction medium for about 5 days.

7. The method of claim 1, wherein culturing the neural ectoderm lineage cells comprises culturing the neural ectoderm lineage cells in a low attachment plate.

8. The method of claim 1, wherein the neural ectoderm lineage cells are derived from cells comprising a cell selected from the group consisting of: induced pluripotent stem cells, embryonic stem cells, and somatic stem cells.

9. A human cornea organoid formed by a method comprising the method of claim 1.

10. A composition comprising one or more cells derived from the human cornea organoid of claim 9.

11. A method for treating a subject comprising applying the composition of claim 10 to an eye of a subject.

12. The composition of claim 11, wherein the one or more cells comprises a cell selected from the group consisting of: an epithelial cell, an epithelial-like cell, a VEGFR1 negative cell, and a stromal cell.

13. A composition comprising extracellular matrix derived from one or more human cornea organoids.

14. The composition of claim 13, wherein the extracellular matrix comprises one of the group consisting of: collagen I, collagen III, collagen V, fibronectin, glycosaminoglycans, keratan sulfate, and chondroitin sulfate.

15. The composition of claim 13, wherein the extracellular matrix comprises a decellularized human cornea organoid.

16. The composition of claim 13, further comprising fibrin gel or polyethylene glycol diacrylate (PEGDA) hydrogel.

17. A method for treating a subject comprising applying the composition of claim 13 to an eye of a subject.

18. The method of claim 17, wherein the subject has one of the diseases or conditions selected from the group consisting of: keratoconus, Fuchs endothelial dystrophy, infection-related corneal scarring, injury-related corneal scarring, a congenital corneal dystrophy, lattice corneal dystrophy, and Avellino corneal dystrophy.

19. A cell culture substrate comprising a coating comprising the composition of claim 13.

20. A method for culturing cells comprising the step of contacting one or more cells to the cell culture substrate of claim 19.

21. A method for sorting human corneal organoid cells comprising the steps of:
contacting the human corneal organoid cells to a cell culture substrate comprising laminin; and
collecting the human corneal organoid cells that are not adhered the cell culture substrate.

22. A method for diagnosing a subject as having, being at risk of having, or being at risk of developing Keratoconus (KC) comprising:
a. measuring the level of an NRF2 target protein or RNA transcript encoding an NRF2 target protein in a biological sample from the subject;
b. comparing the level of the NRF2 target protein or RNA transcript encoding an NRF2 target protein in the biological sample from the subject to the level of NRF2 target protein or RNA transcript encoding an NRF2 target protein in a comparator, wherein a differential level of NRF2 target protein or RNA transcript encoding an NRF2 target protein in the biological sample relative to the comparator indicates that the subject has, or is at risk for developing KC; and
c. diagnosing the subject as having, being at risk of having, or being at risk of developing KC.

23. The method of claim 22, wherein the NRF2 target protein is one or more selected from the group consisting of glutathione peroxidase 3 (GPX3) and malondialdehyde (MDA).

24. The method of claim 22, wherein an increase in the level of the NRF2 target protein or RNA transcript encoding an NRF2 target protein in the biological sample relative to the comparator indicates that the subject has, or is at risk for developing KC.

25. The method of claim 22, wherein the biological sample is at least one selected from the group consisting of tear fluid, epithelial cells, corneal stromal fibroblasts plasma, and serum.

26. The method of claim 22, further comprising a step of treating the subject to treat or prevent KC.

27. The method of claim 22, wherein the step of treating the subject comprises administering to the subject an effective amount of a therapeutic composition comprising an agent that reduces oxidative stress in the eye.

28. The method of claim 27, wherein the step of treating the subject comprises administering to the subject an effective amount of a therapeutic composition that increases the expression of NRF2 or an NRF2 target protein in the eye.

29. The method of claim 28, wherein the NRF2 target protein is selected from HMOX1 and GPX3.
